SEQUENCE LISTING (40 sequences)

SEQ ID NO: 1 - DNA, 1565 bp, Megasphaera elsdenii
atggagagtttgatcctggctcaggacgaacgctggcggcgtgcttaacacatgcaagtcgaacgagaagagatgagaagcttgcttcttatcgattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcgagttaagtcggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacctcctttcta

SEQ ID NO: 2 - DNA, 1563 bp, Lactobacillus ruminis
taaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgttcatgtaaaagacggcttttgctgtcacttttggatgggcctgcggcgtattaacttgttggtggggtaacggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcgagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctccttt

SEQ ID NO: 3 - DNA, 1568 bp, Dialister invisus
atggagagtttgatcctggctcaggacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgcggctggatcacctcctttcta

SEQ ID NO: 4 - DNA, 1538 bp, Bifidobacterium dentium
ttttatggagggttcgattctggctcaggatgaacgctggcggcgtgcttaacacatgcaagtcgaacgggatcccgggggttcgctcccgggtgagagtggcgaacgggtgagtaatgcgtgaccgacctgccccatacaccggaatagctcctggaaacgggtggtaatgccggatgctccggttggatgcatgtccttccgggaaagattccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaaccccttgcgggatggagccgtctaaggtgaggctcgtgattgggactaagtcgtaacaaggtagccgtaccggaaggtgcggctggatcacctcctttctacgga

SEQ ID NO: 5 - DNA, 1497 bp, Eubacterium siraeum
agagtttgatcctggctcaggacgaacgctggcggcgcgcctaacacatgcaagtcgaacggtgaagaggagcttgctcctcggatcagtggcggacgggtgagtaacacgtgagcaacctggctctaagagggggacaacagttggaaacgactgctaataccgcataacgtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacgggaggcagcagtgggggatattggacaatgggggcaaccctgatccagcgacgccgcgtgagggaagaaggttttcggattgtaaacctctgttgacggagaaaaaaatgatggtatccgtttagaaagccacggctaactacgtgccagcagccgcggtaatacgtaggtggcaagcgttgtccggaattactgggtgtaaagggagtgtaggcgggatatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctgggcttttactgacgctgaggctcgaaagcgtggggagcaaacaggattagataccctggtagtccacgccgtaaacgatgattactaggtgtggggggattgaccccttccgtgccggagtaaacacaataagtaatccacctggggagtacgaccgcaaggttgaaactcaaaggaattgacgggggcccgcacaagcagtggagtatgtggtttaattcgacgcaacgcgaagaaccttaccaggtcttgacatcgagtgaccgcctaagagattaggctttcccttcggggacacaaagacaggtggtgcatggttgtcgtcagctcgtgtcgtgagatgttgggttaagtcccgcaacgagcgcaacccttatcattagttgctacgcaagagcactctaatgagactgccgttgacaaaacggaggaaggtggggatgacgtcaaatcatcatgccctttatgacctgggctacacacgtactacaatggcgtttaacaaagagaagcaaagccgcgaggcagagcaaatctccaaaaaacgtctcagttcggattgtaggctgcaactcgcctacatgaagtcggaattgctagtaatcgtaggtcagcatactacggtgaatacgttcccgggccttgtacacaccgcccgtcaaaccatgagagttggcaacacccgaagtcggtagtctaaccgcaaggaggacgccgccgaaggtggggttgatgattagggttaagtcgtaacaaggtagccgtatcggaaggtgcggctggatcacct

SEQ ID NO: 6 - DNA, 1562 bp, Megamonas funiformis
tccttagaaaggaggtgatccagccgcaccttccgatacggctaccttgttacgacttcaccccaatcatcgcccccaccctagacggtcggtaccataggcccttagccggcttcgggtgtgaatgactttcgtggtgtgacgggcggtgtgtacaaggcccgggaacgtattcaccgcagtatgctgacctgcgattactagcgattccgacttcatgtaggcgagtttcagcctacaatccgaactgagagatgcttttagggatccgctccacctcgcgatttcgcttccctctgttcatcccattgtagtacgtgtgtagcccaagacgtaaggggcatgatgacttgacgtcatccccgccttcctccgcgttgtccgcggcagtctactatgagttcccatccgaaatgctggcaacatagtataggggttgcgctcgttgcgggacttaacccaacatctcacgacacgagctgacgacagccatgcaccacctgttttcttgttctccgaagagaaggaacttatctctaagtctttcaatcaatgtcaagtcttggtaaggttcttcgcgttgcgtcgaattaaaccacatactccaccgcttgtgcgggcccccgtcaattcctttgagtttcaaccttgcggccgtactccccaggcgggatacttattgcgttaactccggcacggaaggagtcgatacctcccacacctagtatccatcgtttacggctaggactaccggggtatctaatcccgttcgctaccctagctttcgagcctcagcgtcagttacagtccagaaagccgccttcgccactggtgttcttcccaatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaacaaaaaccgtattataatccttacccttcgtccctaacaacagaactttacgatcctaagaccttcatcgttcacgcggcgttgctccgtcaggctttcgcccattgcggaagattccccactgctgcctcccgtaggagtctgggccgtgtctcagtcccaatgtggccgttcatcctctcagaccggctactgatcgtcgccttggtgggcctttaccctcaccaactagctaatcagacgcaagctcatcttcaagcggaagcttatgtcagaggcctcctttagtaagtgtctcatgcgatactcttacattcattcggtattagcacccctttcgaagtgttgtccccatcttgaaggtagattgcttacgcgttactcacccgttcgccacttggtgtttaccgaaataaacaccccgttcgacttgcatgtgttaagcacgccgccagcgttcgtcctgagccaggatcaaactctccaaaaa

SEQ ID NO: 7 - DNA, 1530 bp, Oscillibacter valericigenes
tttatagagagtttgatcctggctcaggacgaacgctggcggcgtgcttaacacatgcaagtcgaacggagcacccttgattgaggtttcggccaaatgagaggaatgcttagtggcggactggtgagtaacgcgtgaggaacctgcctttcagagggggacaacagttggaaacgactgctaataccgcatgatacatttgggcgacatcgcttgaatgtcaaagatttatcgctgaaagatggcctcgcgtctgattagatagttggtgaggtaacggcccaccaagtcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagtagaagacggtaccccttgaataagccacggctaactacgtgccagcagccgcggtaatacgtaggtggcaagcgttgtccggatttactgggtgtaaagggcgtgtagccgggaaggtaagtcagatgtgaaatctgggggctcaacctccaaactgcatttgaaactacttttcttgagtatcggagaggtaatcggaattccttgtgtagcggtgaaatgcgtagatataaggaagaacaccagtggcgaaggcggattactggacgacaactgacggtgaggcgcgaaagcgtggggagcaaacaggattagataccctggtagtccacgctgtaaacgatcaatactaggtgtgcggggactgaccccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccaggacttgacatcctactaacgaggtagagatacgtcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaagccgtgaggtggagcgaacccctaaaagccgtctcagttcggatcgcaggctgcaactcgcctgcgtgaagtcggaatcgctagtaatcgcggatcagaatgccgcggtgaatacgttcccgggccttgtacacaccgcccgtcacaccatgagagtcgggaacacccgaagtccgtagcctaacagcaatgagggcgcggccgaaggtgggtttgataattggggtgaagtcgtaacaaggtagccgtatcggaaggtgcggctggatcacctccttt

SEQ ID NO: 8 - DNA, 1369 bp, Hafnia alvei
tgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcacgtcatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttacctaaaga

SEQ ID NO: 9 - DNA, 1523 bp, Eubacterium limosum
tttattgagagtttgatcctggctcaggacgaacgctggcggtatgcttaacacatgcaagtcgaacgagaaagttttggtggatccttcgggtgacattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatcacctccttt

SEQ ID NO: 10 - DNA, 1552 bp, Megasphaera elsdenii
agagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacct

SEQ ID NO: 11 - DNA, 1075 bp, Megasphaera sp.
cggtcaatacccgttatccctgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcactaagggtggggactcagaagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaaggaaccagccgtcgaaggtgggggcgatgattggggtgaagtcgtaacaaggtagccgtatcggaaggtgcggctgga

SEQ ID NO: 12 - DNA, 1551 bp, Megasphaera hexanoica
agagtttgatcctggctcaggacgaacgctggcggcgtgcttaacacatgcaagtcgaacgagaggacatggaaagcttgctttttgtggaatcgagtggcaaacgggtgagtaacgcgtaagcaacctgccctgcagatggggacaacagctggaaacggctgctaataccgaatacgaagcagctcccgcatggggggcagcttgaaaggatggcctctgaatatgctatcgctgcaggaggggcttgcgtctgattagctggttggaggggtaacggcccaccaaggcgacgatcagtagccggtctgagaggatgaacggccacattgggactgagacacggcccagactcctacgggaggcagcaggtggggaatcttccgcaatggacgaaagtctgacggagcaacgccgcgtgagtgaagacggccttcgggttgtaaagctctgttatacgggacgaacgggcgggatgctaataccatcctgttgtgacggtaccgtaagagaaagccacggctaactacgtgccagcagccgcggtaatacgtaggtggcaagcgttgtccggaattattgggcgtaaagggtgcgcaggcggtctcttaagtctgtcttaaaagtgcggggctcaaccccgtgaggggacggaaactgggagactggagtatcggagaggaaagcagaattcctagtgtagcggtgaaatgcgtagatattaggaggaataccagtggcgaaagcggctttctggacgacaactgacgctgaggcacgaaagccaggggagcgaacgggattagataccccggtagtcctggccgtaaacgatgggtactaggtgtaggaggtatcgaccccttctgtgccggagttaacgcaataagtaccccgcctggggagtacggccgcaaggctgaaactcaaaggaattgacgggggcccgcacaagcggtggagtatgtggtttaattcgacgcaacgcgaagaaccttaccaagccttgacattgatcgcaatggatagagatatccagttcctcttcggaggacgagaaaacaggtggtgcacggctgtcgtcagctcgtgtcgtgagatgttgggttaagtcccgcaacgagcgcaacccctatcttctgttaccagcacgtgatggtggggactcaggagagactgccgcagacaatgcggaggaaggcggggatgacgtcaagtcatcatgccccttatggcttgggctacacacgtactacaatggctcttaatagagggaagcgaaagggcgacctggagcaaaccccaaaaacagagtcccagttcggattgcaggctgcaacccgcctgcatgaaggaggaatcgctagtaatcgcaggtcagcatactgcggtgaatacgttcccgggccttgtacacaccgcccgtcacaccacgaaagtcattcacacccgaagccggtgaggcaaccgcaaggaaccagccgtcgaaggtgggggcgatgattggggtgaagtcgtaacaaggtagccgtatcggaaggtgcggctgga

SEQ ID NO: 13 - DNA, 1437 bp, Eubacterium hallii
ggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgccgctaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggagctgtcgaaggtggagccggtaactggggtgaagtcgtaacaaggtagccgtatcggaaggtgcggctggatcacctcctttctaaggaa

SEQ ID NO: 14 - DNA, 1499 bp, Acetoanaerobium sticklandii
agagtttgatcctggctcaggatgaacgctggcggcgtgcttaacacatgcaagtcgagcgagcaattcacttcggtaaattgctagcggcggacgggtgcgtaacacgtgggtaacctgcccctatcactgggataacacactgaaaagtgtgctaataccagataacataagcttttggcatcattagcttatcaaagaatttcggatagggatgggcccgcgtctgattagcttgttggtgaggtaacggctcaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctatgggaagataatgacggtaccataggaggaagccccggctaactacgtgccagcagccgcggtaatacgtagggggcaagcgttatccggaattactgggcgtaaagggtgcgtaggcggtttatcaagtcaggggtgaaaggctacggctcaaccgtagtgagcctctgaaactggtgaacttgagtgtaggagaggaaagtggaattcccagtgtagcggtgaaatgcgtagatattgggaggaacaccagtggcgaaggcgactttctggactacaactgacgctgaggcacgaaagcgtggggagcaaacaggattagataccctggtagtccacgctgtaaacgatgagtactaggtgtcggctgtcaaaggtcggtgccggcgttaacacattaagtactccgcctggggagtacgctcgcaagagtgaaactcaaaggaattgacggggacccgcacaagtagcggagcatgtggtttaattcgaagcaacgcgaagaaccttacctaggcttgacatccctttgcaaagcccttaaccgggctcctcttgttcgcaagacaaaggtgacaggtggtgcatggttgtcgtcagctcgtgtcgtgagatgttgggttaagtcccgcaacgagcgcaacccctatttttagttgccatcagttaggctgggcactctagaaagactgccgaggacaactcggaggaaggtggggatgacgtcaaatcatcatgccccttatgcttagggctacacacgtgctacaatggctgttacaaagggctgcgagaccgcgaggtggagccaatcccagaaaaacagtctaagttcggattgtaggctgaaactcgcctacatgaagccggagttactagtaatcgcagatcagaatgctgcggtgaatgcgttcccgggtcttgtacacaccgcccgtcacaccatggaagttgggggcacccaaagttagttatccaaccttacgggggaggctacctaaggtgaattcaatgactggggtgaagtcgtaacaaggtagccgtatcggaaggtgcggctggatcacct

SEQ ID NO: 15 - DNA, 1521 bp, Ruminococcaceae bacterium
tattttagagagtttgatcctggctcaggacgaacgctggcggcgtgcctaacacatgcaagtcgaacgaaactttttgcttcggtagaaagtttagtggcggacgggtgagtaacgcgtgaggaacctgcctttcagagggggataatgtctggaaacggacactaataccgcatgacattttctgttcacatggacagaaaatcaaaggagcaatctgctgaaagatggactcgcgtccgattagctagatggtgagataatagcccaccatggcgacgatcggtagccggactgagaggttgaacggccacattgggactgagacacggcccagactcctacgggaggcagcagtgggggatattgcacaatggaggaaactctgatgcagcaacgccgcgtgaaggaagacggtcttcggattgtaaacttttgtacctagggacgataatgacggtacctaggcagcaagctccggctaactacgtgccagcagccgcggtaatacgtagggagcgagcgttgtccggatttactgggtgtaaagggtgcgtaggcggccaagcaagtcagctgtgaaaactatgggcttaacccatagcctgcaattgaaactgtttggcttgagtgaagtagaggtaggtggaattcccggtgtagcggtgaaatgcgtagagatcgggaggaacaccagtggcgaaggcgacctactgggctttaactgacgctgaagcacgaaagcatgggtagcaaacaggattagataccctggtagtccatgctgtaaacgatgattactaggtgtggggggtctgaccccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggctacacacgtactacaatggccgttaacagagagaagcgataccgcgaggtggagcgaacctcaaaaagcggtctcagttcggattgcaggctgaaacccgcctgcatgaagttggaattgctagtaatcgcggatcataatgccgcggtgaatacgttcccgggccttgtacacaccgcccgtcacaccatgggagccggtaatacccgaagtcagtagtctaaccgcaaggaggacgctgccgaaggtaggattggcgactggggtgaagtcgtaacaaggtagccgtatcagaaggtgcggctggatcacctcctttctatgga

SEQ ID NO: 16 - DNA, 1506 bp, Clostridium kluyveri
agagtttgatcctggctcaggacgaacgctggcggcgtgcctaacacatgcaagtcgagcgaggaagccccttcgggggtggaatagcggcggacgggtgagtaacacgtgggtaacctgcctcaaagagggggatagcctcccgaaagggagattaataccgcatagaaggtaaaaatcgcatgataaatgccttaaaggagcaatccgctttgagatgggcccgcgtcgcattagctagttggagggataaaagccccccaaggcgacgatgcgtagccgacctgagagggtgaacggccacattggaactgagatacggtccagactcctacgggaggcagcagtggggaatattgcacaatgggggaaaccctgatgcagcaacgccgcgtgagtgaagaaggttttcggatcgtaaagctctgtcatccgggacgataatgacggtaccggaagaggaagccacggctaactacgtgccagcagccgcggtaatacgtaggtggcgagcgttgtccggaattactgggcgtaaagggtgtgcaggcggatatttaagtgagatgtgaaagacccgggcttaacccgggcagtgcatttcaaactggatatctagagtgcaggagaggagaacggaattcctagtgtagcggtgaaatgcgtagagattaggaagaacaccagtggcgaaggcggttctctggactgtaactgacgctgaggcacgaaagcgtgggtagcaaacaggattagataccctggtagtccacgccgtaaacgatgagtactaggtgtagggggtatcgaccccccctgtgccgcagtaaacacaataagtactccgcctgggaagtacgatcgcaagattaaaactcaaaggaattgacggggacccgcacaagcagcggagcatgtggtttaattcgaagcaacgcgaagaaccttacctggacttgacatcccctgaatgactcgtaatggaggaagccctacggggcagggagacaggtggtgcatggttgtcgtcagctcgtgtcgtgagatgttaggttaagtcctgcaacgagcgcaacccctgtcgttagttgccagcacgtaaaggtgggcaccctaacgagactgccgcggttaacgtggaggaaggtggggatgacgtcaaatcatcatgccccttatgtccagggcaacacacgtgctacaatgggcagaacagagagaagcaataccgcgaggaggagcaaatctcaaaaactgcccccagttcggattgcaggctgaaacccgcctgcatgaagttggagttgctagtaatcgcgaatcagcatgtcgcggtgaatacgttcccgggtcttgtacacaccgcccgtcacaccatgagagctggcaacacccgaagtccgtagtctaaccaaggaggacgcggccgaaggtggggttagtgattggggtgaagtcgtaacaaggtagccgtaggagaacctgcggctggatcacctcctttct

SEQ ID NO: 17 - DNA, 1453 bp, Coprococcus comes
gatgaacgctggcggcgtgcttaacacatgcaagtcgaacgaagcacttatmcttgattcttcggatgaagrggtttgtgactgagtggcggacgggtgagtaacgcgtgggtaacctgcctcatacagggggataacagttagaaatgactgctaataccgcataagaccacrgagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtg

SEQ ID NO: 18 - DNA, 1440 bp, Coprococcus catus
gatgaacgctggcggcgtgcttaacacatgcaagtcgaacggacgatgaagagcttgcttttcaragttagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacaggattagataccctggtagtccacgccgtaaacgatgaatactaggtgtcggggsycataagrsyttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggccttgacatcccggtgaccgtccmgtaatggggacytcycttcggagcaccggwgacaggtggtgcatggttgtcgtcagctcgtgtcgtgagatgttgggttaagtcccgcaacgagcgcaacccctatgttcagtagccagcaggtaaggctgggcactctggacagactgccggggataacccggaggaaggcggggatgacgtcaaatcatcatgccccttacggcctgggctacacacgtgctacaatggcgtaaacaaagggaagcgagagggtgacctggagcgaatcccaaaaataacgtcccagttcggactgtagtctgcaacccgactacacgaagctggaatcgctagtaatcgcgaatcagcatgtcgcggtgaatacgttcccgggtcttgtacacaccgcccgtcacaccatgggagttggaaatgcccgaagtcagtgacctaaccgcaagggaggagctgccgaaggtggacccgatgactggggtg

SEQ ID NO: 19 - DNA, 1453 bp, Roseburia inulinivorans
ttgatcctggctcaggatgaacgctggcggcgtgcttaacacatgcaagtcgaacgaagcacttttacgatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggacagtaatgtgtcctttccttcgggacagaagtgacaggtggtgcatggttgtcgtcagctcgtgtcgtgagatgttgggttaagtcccgcaacgagcgcaacccttatccccagtagccagcggttcggacgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgcgaaggcaggt

SEQ ID NO: 20 - DNA, 1545 bp, Lactobacillus ruminis
agagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgtttatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacatgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctgga

SEQ ID NO: 21 - DNA, 1352 bp, Lactobacillus rhamnosus
aggatgaacgctggcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaaaaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgacggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatc

SEQ ID NO: 22 - DNA, 1563 bp, Lactobacillus reuteri
agagtttgatcctggctcaggatgaacgccggcggtgtgcctaatacatgcaagtcgtacgcactggcccaactgattgatggtgcttgcacctgattgacgatggattaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagcaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttattagttgccagcattaagttgggcactctagtgagactgccggtgacaaaccggaggaaggtggggacgacgtcagatcatcatgccccttatgacctgggctacacacgtctacaatggacggtacaacgagtcgcaagctcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggagggagccgcctaaggcgggacagatgactggggtgaagtcgtaacaaggtagccgtaggagaacctgcggctggatcacct

SEQ ID NO: 23 - DNA, 1549 bp, Lactobacillus plantarum
agagtttgatcctggctcaggacgaacgctggcggcgtgcctaatacatgcaagtcgaacgaactctggtattgattggtgcttgcatcgtgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctgga

SEQ ID NO: 24 - DNA, 1545 bp, Lactobacillus salivarius
gagagtttgatcctggctcaggacgaacgctggcggcgtgcctaatacatgcaagtcgaacgaaactttcttacaccgaatgcttgcattcaccgtaagaagttgagtggcggacgggtgagtaacacgtgggtaacctgcctaaaagaaggggataacacttggaaacaggtgctaataccgtatatctctaaggatcgcatgattcttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcgagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctgga

SEQ ID NO: 25 - DNA, 1551 bp, Dialister succinatiphilus
agagtttgatcctggctcaggacgaacgctggcggcgtgcttaacacatgcaagtcgaacgagaggacaagagaagcttgcttcttttggaatcgagtggcaaacgggtgagtaacacgtaaacaacctgccttcaggatggggacaacagacggaaacgactgctaataccgaatacacttggaagaccgcatgatcttccaaggaaagggtggcctctacctgtaagctatcgcctgaagaggggtttgcgtctgattaggcagttggtgaggtaacggcccaccaaacctacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaagctctgtgatcggggacgaacggcccgtaagctaataccttatggaagtgacggtacccgaatagcaagccacggctaactacgtgccagcagccgcggtaatacgtaggtggcaagcgttgtccggaattattgggcgtaaagcgcgcgcaggcggctttctaagtccatcttaaaagtgcggggcttaaccccgtgatgggatggaaactggaaagctggagtatcggagaggaaagtggaattcctagtgtagcggtgaaatgcgtagagattaggaagaacaccggtggcgaaggcgactttctggacgacaactgacgctgaggcgcgaaagcgtggggagcaaacaggattagataccctggtagtccacgccgtaaacgatgaatactaggtgtaggaggtatcgaccccttctgtgccggagctaacacaataagtattccgcctgggaagtacgatcgcaagattaaaactcaaaggaattgacgggggcccgcacaagcggtggagtatgtggtttaattcgacgcaacgcgaagaaccttaccaggtcttgacattgatcgccattcacagaaatgtgaagttctccttcgggagacgagaaaacaggtggtgcacggctgtcgtcagctcgtgtcgtgagatgttgggttaagtcccgcaacgagcgcaacccctatcttatgttaccagcacgttatggtggggactcatgagagaccgccgcggacaacgcggaggaaggtggggatgacgtcaagtcatcatgccccttatgacctgggctacacacgtactacaatgggtgtcaacaaagagaagcaaaaccgcgaggtggagcgaacctcaaaaacacacccccagttcagattgcaggctgcaacccgcctgcatgaagtaggaatcgctagtaatcgcgggtcagcataccgcggtgaatacgttcccgggccttgtacacaccgcccgtcacactatgagagtcggaaacacccgaagccggtgaggtaaccgcaaggagccagccgtcgaaggtggagctgatgattggagtgaagtcgtaacaaggtagccgtatcggaaggtgcggctgga

SEQ ID NO: 26 - DNA, 1513 bp, Bifidobacterium dentium
agggttcgattctggctcaggatgaacgctggcggcgtgcttaacacatgcaagtcgaacgggatcccgggggttcgcctccgggtgagagtggcgaacgggtgagtaatgcgtgaccgacctgccccatacaccggaatagctcctggaaacgggtggtaatgccggatgctccggttggatgcatgtccttccgggaaaggttccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggccgtagagatacggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaacccttctgtgggatggagccgtctaaggtgaggctcgtgattgggactaagtcgtaacaaggtagccgtaccggaaggtgcggctgga

SEQ ID NO: 27 - DNA, 1519 bp, Bifidobacterium dentium
agggttcgattctggctcaggatgaacgctggcggcgtgcttaacacatgcaagtcgaacgggatcccgggggttcgcctccgggtgagagtggcgaacgggtgagtaatgcgtgaccgacctgccccatacaccggaatagctcctggaaacgggtggtaatgccggatgctccggttggatgcatgtccttccgggaaaggttccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggccgtagagatacggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaacccttctgtgggatggagccgtctaaggtgaggctcgtgattgggactaagtcgtaacaaggtagccgtaccggaaggtgcggctggatcacct

SEQ ID NO: 28 - DNA, 1510 bp, Bifidobacterium adolescentis
agggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttgaccgcatggtcctctgggaaagcttttgcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacaccgcgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatc

SEQ ID NO: 29 - DNA, 1515 bp, Bifidobacterium adolescentis
agggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacct

SEQ ID NO: 30 - DNA, 1543 bp, Mitsuokella multiacidus
agagtttgatcctggctcaggacgaacgctggcggcgtgcttaacacatgcaagtcgaacgagacgattgaaagcttgcttttgaaagtcgagtggcaaacgggtgagtaacgcgtagacaacctgccgtaaagatggggacaacagtccgaaaggactgctaataccgaatgttgtgggacttccgcatgggagttctactaaagatggcctctacttgtaagctatcgctttacgatgggtctgcgtctgattagctagttggtggggtaacggcttaccaaggcgacgatcagtagccggtctgagaggatgaacggccacattggaactgagacacggtccagactcctacgggaggcagcagtggggaatcttccgcaatgggcgcaagcctgacggagcaacgccgcgtgagtgaagaagggtttcgactcgtaaagctctgttgtcggggacgaatgtggagatggtgaataaccatttccaatgacggtacctgacgaggaagccacggctaactacgtgccagcagccgcggtaatacgtaggtggcgagcgttgtccggaattattgggcgtaaagggagcgcaggcgggaggtcaagtctatcttaaaagtgcggggctcaaccccgtgaggggatggaaactggtcttcttgagtgcaggagaggaaagcggaattcctagtgtagcggtgaaatgcgtagatattaggaggaacaccagtggcgaaggcggctttctggactgtaactgacgctgaggctcgaaagcgtggggagcgaacaggattagataccctggtagtccacgccgtaaacgatgaatgctaggtgtaggaggtatcgacccctcctgtgccggagttaacgcaataagcattccgcctggggagtacggccgcaaggctgaaactcaaaggaattgacgggggcccgcacaagcggtggagtatgtggtttaattcgacgcaacgcgaagaaccttaccagggcttgacattgagtgaaaggactagagatagtcccctctcttcggagacacgaaaacaggtggtgcatggctgtcgtcagctcgtgtcgtgagatgttgggttaagtcccgcaacgagcgcaacccctatcctttgttgccagcacataatggtgggaactcaaaggagactgccgcggacaacgcggaggaaggcggggatgacgtcaagtcatcatgccccttatgtcctgggctacacacgtactacaatgggatggacagagagcagcgaagccgcgaggccaagcgaaccccataaaccatctcccagttcggattgcaggctgcaacccgcctgcatgaagttggaatcgctagtaatcgcaggtcagcatactgcggtgaatacgttcccgggccttgtacacaccgcccgtcacaccacggaagtcattcacacccgaagccggcaggctaaccgcaaggaggcagccgtctaaggtgggggcgatgactggggtgaagtcgtaacaaggtagccgtatcggaaggtgcggctgga

SEQ ID NO: 31 - DNA, 1537 bp, Selenomonas sputigena
agagtttgatcctggctcaggacgaacgctggcggcgtgcttaacacatgcaagtcgaacgggaacctttatttcggtaaaggtgagagtggcaaacgggtgagtaacacgtaggcaacctgccgacaggatggggacaacattccgaaaggaatgctaataccgaatggagtccggggatggcatcatccctggataaaagatggcctctgaatatgctatcgcctgtcgatgggcctgcgtctgattagccagttggcggggtaacggcccaccaaagcgacgatcagtagccggtctgagaggatgaacggccacattgggactgagacacggcccagactcctacgggaggcagcagtggggaatcttccacaatgggcgaaagcctgatggagcaacgccgcgtgagtgaagaaggtcttcggatcgtaaagctctgttgcacgggacgaaaaccgagcttgagaatattgagtttgggtgacggtaccgagcgaggaagccacggctaactacgtgccagcagccgcggtaatacgtaggtggcgagcgttgtccggaattattgggcgtaaagggagcgcaggcggacatataagtccatcttaaaagtgcggggctcaaccccgtgaggggatggaaactgtatgccttgagtgcaggagaggaaagcggaattcccagtgtagcggtgaaatgcgtagatattgggaggaacaccagtggcgaaggcggctttctggactgtaactgacgctgaggctcgaaagccaggggagcgaacgggattagataccccggtagtcctggccgtaaacgatgaatgctaggtgtaggaggtatcgacccctcctgtgccggagttaacgcaataagcattccgcctggggagtacggtcgcaagactgaaactcaaaggaattgacgggggcccgcacaagcggtggagtatgtggtttaattcgacgcaacgcgaagaaccttaccagggcttgacattgagtgaaagagctagagatagcttcctcccttcggggacacgaaaacaggtggtgcatggctgtcgtcagctcgtgtcgtgagatgttgggttaagtcccgcaacgagcgcaacccctgtcctttgttgccagcgcgtaatggcgggaactcaaaggagactgccgcggagaacgcggaggaaggcggggatgacgtcaagtcatcatgccccttatgtcctgggctacacacgtactacaatgggatggacagagggaagcgaaggcgtgagccggagcggaccccacaaaccatcccccagttcggattgcaggctgcaacccgcctgcatgaagtcggaatcgctagtaatcgcaggtcagcatactgcggtgaatacgttcccgggccttgtacacaccgcccgtcacaccacggaagtcattcacgcccaaagccggtgggccaaccgcaaggaggcagctgtctaaggcgggggcgatgactggggtgaagtcgtaacaaggtagccgtatcggaaggtgcggctgga

SEQ ID NO: 32 - DNA, 1646 bp, Phascolarctobacterium succinatutens
agagtttgatcctggctcaggacgaacgctggcggcatgcctaacacatgcaagtcgaacggagaaagttcaacaccaagtatttcatccgctgaagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtgaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaaggtgcggctggatcacct

SEQ ID NO: 33 - DNA, 1557 bp, Phascolarctobacterium faecium
attggagagtttgatcctggctcaggacgaacgctggcggcgtgcctaacacatgcaagtcgaacggagaattttatttcggtagaattcttagtggcgaacgggtgagtaacgcgtaggcaacctgccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctattaggagccagccgtctaaggtggggccgatgattggggtgaagtcgtaacaaggtagccgtatcggaaggtgcggctggatcacctccttt

SEQ ID NO: 34 - DNA, 1513 bp, Oscillibacter sp.
agagtttgatcctggctcaggacgaacgctggcggcgtgcttaacacatgcaagtcgaacggagcacccttgactgaggtttcggccaaatgataggaatgcttagtggcggactggtgagtaacgcgtgaggaacctaccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggaggcaaagccgcgaggcagagcaaacccccaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctgga

SEQ ID NO: 35 - DNA, 1508 bp, Eubacterium callanderi
agagtttgatcctggctcaggacgaacgctggcggtatgcttaacacatgcaagtcgaacgagaaggttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcgagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaaggtgcggctggatc

SEQ ID NO: 36 - DNA, 1509 bp, Acetobacterium woodii
agagtttgatcctggctcaggacgaacgctggcggtatgcttaacacatgcaagtcgaacgagacaagatggaatgatccttcgggtgaatgaaatcttagaaagtggcgaacgggtgagtaacgcgtgggtaacctaccctatggaaaggaatagccccgggaaactgggtgtaataccttataagatatatttgtcgcatggcagatgtattaaacgctccggcgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggaaaccctgacgcagcaataccgcgtgagtgaagaaggttttcggatcgtaaagctctgttattggggaagaaaaaaagacggtacccaagaagaaagtcccggctaactacgtgccagcagccgcggtaatacgtaggggacaagcgttgtccggatttactgggcgtaaagggcacgcaggcggttttttaagtcagatgtgaaaggtaccggctcaaccggtgacatgcatttgaaactgaaagacttgagtattggagaggcaagtggaattcctggtgtagcggtgaaatgcgtagatatcaggaggaacaccggtggcgaaggcggcttgctggacaaatactgacgctgaggtgcgaaagcgtggggagcaaacaggattagataccctggtagtccacgccgtaaacgatgagtgctaggtgttggggagactcagtgccgcagctaacgcaataagcactccgcctggggagtacgaccgcaaggttgaaactcaaaggaattgacggggacccgcacaagcagcggagcatgtggtttaattcgaagcaacgcgaagaaccttaccaggtcttgacatcctctgaccatctgagagatcagactttcccttcggggacagagagacaggtggtgcatggttgtcgtcagctcgtgtcgtgagatgttgggttaagtcccgcaacgagcgcaacccctgtggttagttgccatcatttagttgggcactctaagcagactgccgtggataacacggaggaaggtggggacgacgtcaaatcatcatgccccttatgacctgggctacacacgtgctacaatggtctgaacagagggctgcgaaaccgcgaggtgaagctaatcccttaaaacagatctcagttcggattgcaggctgcaactcgcctgcatgaagttggagttgctagtaatcgcagatcagaatgctgcggtgaatgcgttcccgggtcttgtacacaccgcccgtcacaccacgagagttggcaacacccgaagtcagtgaggcaaccgtaaggagccagctgccgaaggtggggtcagtaattggggtgaagtcgtaacaaggtagccgtatcggaaggtgcggctggatc

SEQ ID NO: 37 - DNA, 1520 bp, Blautia producta
agagtttgatcctggctcaggatgaacgctggcggcgtgcttaacacatgcaagtcgagcgaagcgctaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaatagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacggggacttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacct

SEQ ID NO: 38 - DNA, 1474 bp, Eubacterium callanderi
cgaacgctggcggtatgcttaacacatgcaagtcgaacgagaaagttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggatgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactcagcagtcgaaggtggggctagtaattggggtgaagtcgtaacaaggtagccgtatcggaagg

SEQ ID NO: 39 - DNA, 1431 bp, Eubacterium limosum
atgcttaacacatgcagtcgaacgagaaggttttgatggatccttcgggtgacattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgcaaggactccaggcagcagtcgaaggtggggtaacaaggtaacc

SEQ ID NO: 40 - DNA, 1517 bp, Blautia hydrogenotrophica
gagagtttgatcctggctcaggatgaacgctggcggcgtgcttaacacatgcaagtcgaacgaagcgatagagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagaaagtgacggtacctgactaagaagccccggctaattacgtgccagcagccgcggtaatacgtaaggggcaagcgttatccggatttactgggtgtaaagggagcgtagacggtttggcaagtctgatgtgaaaggcatgggctcaacctgtggactgcattggaaactgtcagacttgagtgccggagaggcaagcggaattcctagtgtagcggtgaaatgcgtagatattaggaggaacaccagtggcgaaggcggcctgctggacggtaactgacgttgaggctcgaaagcgtggggagcaaacaggattagataccctggtagtccacgctgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggaagtaatgttcccttttcttcggaacagaggagacaggtggtgcatggttgtcgtcagctcgtgtcgtgagatgttgggttaagtcccgcaacgagcgcaacccttattcttagtagccagcaggtagagctgggcactctagggagactgccagggataacctggaggaaggtggggatgacgtcaaatcatcatgccccttatgatttgggctacacacgtgctacaatggcgtaaacaaagggaagcgaaggggtgacctggagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgaaaggagggagctgccgaaggtgggactgataactggggtgaagtcgtaacaaggtagccgtatcggaaggtgcggctgga

(19) United States
(12) Patent Application Publication
(10) Pub. No.: US 2024/0226195 A1
(43) Pub. Date: Jul. 11, 2024

(54) MICROBIAL CONSORTIUM AND USES THEREOF

(71) Applicant: BIOMICA LTD., Rehovot (IL)

(72) Inventors: Sheerli KRUGER BEN SHABAT, Raanana (IL); Yehuda RINGEL, Tel Aviv (IL); Shiri MESHNER, Raanana (IL); Elran HABER, Kiryat Ono (IL); Shiri ESHAR, Hulda (IL); Osnat TIROSH, Ness Ziona (IL); Omri POLONSKI, Kibbutz Givat Brenner (IL)

(73) Assignee: BIOMICA LTD., Rehovot (IL)

(21) Appl. No.: 18/290,284

(22) PCT Filed: May 12, 2022
(86) PCT No.: PCT/IL2022/050503

§ 371 Date: Nov. 10, 2023

(30) Foreign Application Priority Data
May 12, 2021 (IL) 283124

(51) Int. Cl. A61K 35/745; A61K 35/741; A61P 1/00; C12N 1/20; C12R 1/01

(52) U.S. Cl. CPC A61K 35/745; A61K 35/741; A61P 1/00; C12N 1/205; C12R 2001/01

(57) ABSTRACT

The present invention provides a microbial consortium comprising two or more microorganisms, compositions and kits comprising the same and uses thereof in methods of treating functional gastrointestinal disorders.

### TECHNOLOGICAL FIELD

The present discourse relates to consortium of microorganisms, compositions and kits comprising the same and uses thereof.

### BACKGROUND ART

References considered to be relevant as background to the presently disclosed subject matter are listed below:
International patent application publication No. WO18109461.
Zhu S, Liu S, Li H, Zhang Z, Zhang Q, Chen L, Zhao Y, Chen Y, Gu J, Min L, Zhang S. Identification of Gut Microbiota and Metabolites Signature in Patients with Irritable Bowel Syndrome. Front Cell Infect Microbiol. 2019; 9:346.
US patent application No. 2020/206280
Pokusaeva K, Johnson C, Luk B, Uribe G, Fu Y, Oezguen N, Matsunami R. K, Lugo M, Major A, Mori-Akiyama Y, Hollister E. B, Dann S. M, Shi X. Z, Engler D. A, Savidge T, Versalovic J, GABA-producing Bifidobacterium dentium modulates visceral sensitivity in the intestine. Neurogastroenterology & Motility, 2017; 29(1): e12904.
International patent application publication No. WO20202148.

Acknowledgement of the above references herein is not to be inferred as meaning that these are in any way relevant to the patentability of the presently disclosed subject matter.

### BACKGROUND

International patent application publication No. WO18109461 provides compositions comprising a bacterial strain of the genus Blautia, for use in a method of increasing the microbiota diversity and/or inducing stability of the microbiota of a subject.

Zhu S et al describes a study aimed at investigating potential mechanism and novel biomarkers of IBS through evaluation of the metabolomic and microbiologic profile.

US patent application No. 2020/206280 describes compositions comprising bacterial strains for treating and preventing neurodegenerative disorders.

Pokusaeva K, et al demonstrates that commensal Bifidobacterium dentium produces GABA via enzymatic decarboxylation of glutamate by GadB and that daily oral administration of this specific Bifidobacterium strain modulated sensory neuron activity in a rat fecal retention model of visceral hypersensitivity.

International patent application publication No. WO20202148 provides a microbial consortium comprising two or more microorganisms, compositions and kits comprising the same and uses thereof for treating cancer.

### GENERAL DESCRIPTION

In accordance with some aspects, the present disclosure provides a microbial consortium comprising two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with some other aspects, the present disclosure provides a microbial consortium comprising two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof for use in treating a functional gastrointestinal disorder.

In accordance with some further aspects, the present disclosure provides a composition comprising a microbial consortium, the a microbial consortium two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with yet some other aspects, the present disclosure provides a use of a microbial consortium in the preparation of a pharmaceutical composition for treating a functional gastrointestinal disorder, wherein the microbial consortium two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with yet some further aspects, the present disclosure provides a kit composition comprising a microbial consortium and optionally instructions for treating a functional gastrointestinal disorder, wherein the microbial consortium two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are bar graphs showing short chain fatty acids (SCFAs) production by Megasphaera elsdenii ATCC 17753; FIGS. 1A and 1B show SCFAs concentrations after 7 hours of anaerobic growth at 37° C. in De Man, Rogosa and Sharpe (MRS) medium, FIG. 1A, or MRS medium supplemented with 90 mM lactate, FIG. 1B; error bars represent standard error of 2 independently performed experiments; data are expressed as mean; FIG. 1C shows production of isovalerate and valerate after 26 hours of anaerobic growth at 37° C. in MRS medium supplemented with 90 mM lactate; data are expressed as mean.

FIG. 2 is a bar graph showing SCFAs production by Megamonas funiformis YIT 11815; SCFAs concentrations were measured after 30 hours of anaerobic growth at 37° C. in MRS medium, data are expressed as mean.

FIG. 3 is a bar graph showing acetate production by Bifidobacterium dentium ATCC 27679; acetate concentrations were measured after 26 hours of anaerobic growth at 37° C. in MRS medium, data are expressed as mean.

FIG. 4 is a bar graph showing acetate production by Hafnia alvei ATCC 51873; acetate concentrations were measured after 9.5 hours of anaerobic growth at 37° C. in BHI medium, data are expressed as mean.

FIG. 5 is a bar graph showing acetate and butyrate production by Eubacterium limosum ATCC 8486; concentrations were measured after for up to 24 hours of growth, results were expressed in g/ml.

FIG. 6 is a bar graph showing GABA production by Bifidobacterium dentium ATCC 27679, Eubacterium limosum ATCC 8486 and Cetobacterium somerae ATCC BAA-474 as a negative control bacterium; GABA concentrations were measured up to 72 hours of anaerobic growth at 37° C.; error bars represent standard error of 2 independently performed experiments; data are expressed as mean.

FIG. 7 is a graph showing fold change of 3β-hydroxysteroid dehydrogenase (30 HSD) expression level using real-time PCR during growth of Hafnia alvei ATCC 51873 relative to levels of housekeeping genes (HKGs), RecA transcription termination factor rho (Rho), all expression levels are normalized to RecA levels at the beginning of the assay (time 0). relative fold-change is calculated from the first sampling point of Hafnia alvei ATCC 51873 fermentation.

FIG. 8 is a bar graph showing serotonin levels in supernatant of RIN14B cells after treatment with Bifidobacterium dentium ATCC 27679 culture medium, y axis presents nanomol (nmol) of serotonin per total protein concentrations (ug/ml) in samples as quantified by Bradford assay, results are expressed as means±SEM.

FIG. 9 is a bar graph showing effect of exemplary consortia on number of fecal pellets excreted during the 2 h of wrap-restraint stress (WRS)-induced model, results are expressed as means±SEM.

FIG. 10 is a graph showing effect of exemplary consortia on number of normalized Abdominal contractions (AC) measured with balloon inflated to the different volumes in the colo-rectal distension (CRD) test, the number of AC recorded was normalized to the previous AC measured, results are expressed as means±SEM, * p<0.05 in two-way ANOVA, comparison of consortium 12 and vehicle.

FIG. 11 is a bar graph showing effect of exemplary consortia on number of normalized AC measured with balloon inflated to 0.8 ml in the CRD test, the number of AC recorded is normalized to the previous AC measured in the balloon volume of 0.4 ml, results are expressed as means±SEM, * p<0.05 in Student's t-test.

### DETAILED DESCRIPTION OF EMBODIMENTS

The gut microbiome refers to a large number of diverse microorganisms within the gastrointestinal tract that are capable of affecting a variety of physiological process, including maintenance of host's health as well as influencing disease development and progression.

The present disclosure is based on possible connections and correlations between abundance of microorganisms in the gut microbiome, microbial and/or host metabolites, microbial and/or host pathways, as well as interkingdom host-microbiome interactions and conditions associated with the gastrointestinal system and is aimed at using specific microorganisms detected in, isolated from or purified from a microbiome, for example the gut microbiome, in preventing and treating gastrointestinal conditions, specifically functional gastrointestinal conditions.

To that end, the inventors have used an array of computational tools utilized to process high-throughput sequencing data and obtained high resolution detection and annotation of microbial genes and pathways as well as microbial taxa. This resulted in a mechanistic understanding of a relationship between an organism's cellular processes (such as a microorganism) and various human and/or microbial cellular processes related for example to the enteric nervous system and/or the gut-brain axis.

During the analysis, several methods such as stringent statistical comparative analyses, were applied which enabled identification of specific microbial functions (pathways) and selection of specific microorganisms that have significantly differential abundance depending on syndrome severity and specifically differential abundance between patients presenting low-symptomatic gastrointestinal conditions versus patients presenting high-symptomatic gastrointestinal conditions such as functional gastrointestinal conditions.

The differentiation between patients presenting low-symptomatic gastrointestinal conditions and patients presenting high-symptomatic gastrointestinal conditions was done by considering existence/appearance of various symptoms associated with functional gastrointestinal conditions, specifically of Irritable Bowel Syndrome (IBS). Such symptoms include symptoms associated with abdominal discomfort, for example, abdominal bloating, abdominal pain, or associated abnormal with transit time. This physiological information may be obtained by various means, including specific questioners filled by patients diagnosed with functional gastrointestinal condition and analysis of the obtained data.

It was suggested that a unique and specific microorganism identified computationally by the invention, may be administered to a subject, either as each microorganism alone or preferably in specific combinations (denoted herein as microbial consortia), in order to alter the microbiome population and to treat or prevent at least one functional gastrointestinal disorder. It was further suggested that the identified microorganism may be used for diagnostic and prognostic purposes, for example for assessing responsiveness of a subject to treatment and for determining treatment protocols.

As such, the present disclosure provides a microbial consortium. The microbial consortium comprises two or more microorganisms that are collectively capable of modulating different physiological/biological processes (pathways) in the microorganisms and/or in the host.

Surprisingly, it was found that microorganisms which were over-represented in patients characterized with a low-symptomatic functional gastrointestinal condition were capable of modulating a combination of an array of pathways including, inter alia, pathways that affect interkingdom communication between the microorganisms and a host for example pathways that involve the enteric nervous system (ENS) and/or the central nervous system (CNS). As shown in the Examples below, these microorganisms were surprisingly found to modulate a variety of functions, including, inter alia, production of short chain fatty acids, production of GABA, secretion of serotonin, and production of progesterone. The microorganisms were shown to produce short chain fatty acids, including, inter alia, acetate, butyrate, propionate, valerate and isovalerate.

Surprisingly, it shown that growth medium of at least one of the identified microorganisms can induce serotonin production in a host cell. This suggested that components produced by the at least one of the identified microorganisms are secreted to the growth medium, which in turn can affect processes in the host.

Further, it was surprisingly shown that at least one of the identified microorganisms express 3β-hydroxysteroid dehydrogenase (3β-HSD) and the amount of the expressed 3β-HSD was higher in the presence of a progesterone precursor, pregnenolone. These data suggested that at least one of the identified microorganisms is capable of producing progesterone, at least via the activity of 3β-HSD.

Further and as shown below, exemplary microbial consortia comprising identified microorganisms were shown to have a positive effect when tested in vivo. Specifically, as shown in Example 2A, rats treated with three exemplary microbial consortia exhibited a decrease in the number of fecal pellets excreted during the 2 h of WRS and in addition, the number of abdominal contractions episodes were lower in the animals treated with the three exemplary microbial consortia.

Thus, the present disclosure provides in accordance with some aspects, a microbial consortium comprising two or more microorganisms, at least one of the two or more microorganisms having one or more of the following features: (i) capable of modulating visceral hypersensitivity, (ii) capable of modulating intraluminal gas balance, (iii) capable of modulating ENS, (iv) capable of modulating gut-brain axis, (v) capable of modulating v-aminobutyric acid (GABA), (vi) capable of modulating gut-barrier, (vii) capable of modulating pathways associated with low-grade inflammation, (viii) capable of modulating fatty acid production, (ix) capable of modulating dietary compounds metabolism, (x) capable of modulating detoxification activity, (xi) capable of modulating an endopeptidase activity, (xii) capable of modulating serotonin or combination thereof.

It should be noted that at times the above features as well as other features of the microbial consortium described herein below are referred to as the “pathway” and at times the pathway product (direct or indirect) is denoted as “substance”.

It should be also noted that reference to at least one microorganism having one ore more of or at least one of is to be understood as characterization of the at least one microorganism indicating that the at least one microorganism (or one or more) is capable of having and at times having the characteristics (features) as listed.

In the following text, when referring to the microbial consortium it is to be understood as also referring to the pharmaceutical compositions, kits, uses and methods disclosed herein. Thus, whenever providing a feature with reference to the microbial consortium, it is to be understood as defining the same feature with respect to the pharmaceutical compositions, kits, uses and methods, mutatis mutandis.

The microbial consortium as used herein refers to a mixture/cocktail of microorganisms, including at least one of a bacterium and/or an archaea. When referring to at least one microorganism it should be understood as referring to one microorganism species and/or strain as classified under common scientific classification. The microorganisms being the subject of the present disclosure are present in the human microbiome and thus can be isolated and/or purified from any microbiome, such as the human microbiome, by any known method in the art as also detailed below or purified from a biological material (e.g, fecal materials, such as feces or materials isolated from the various segments of the small and large intestines).

As such, the term microorganism used herein refers in accordance with some embodiments, an isolated microorganism, a purified microorganism, a recombinant microorganism or any combinations thereof.

In some embodiments, the microbial consortium comprises isolated microorganisms. In some embodiments, the microbial consortium comprises purified microorganisms. In some embodiments, the microbial consortium comprises recombinant microorganisms. In some embodiments, the microbial consortium comprises isolated microorganisms, purified microorganisms or any combination thereof.

It should be noted that the recombinant microorganisms of the present invention are microorganisms whose genetic makeup has been altered by deliberate introduction or deletion of genetic elements. The recombinant microorganisms may maintain the functions (cellular processes) of the original microorganism.

In some embodiments, the microorganism is a live microorganism, provided as spores, heat-killed, non-living form of the microorganism, an extract of the organism, a component of the microorganism or any combination thereof.

In some embodiments, the microorganism is a live microorganism. In some other embodiments, the microorganism is provided as spores, heat-killed, non-living form of the microorganism. In some further embodiments, the microorganism is an extract of the microorganism. In yet some further embodiments, the microorganism is a component of the microorganism.

Reference to at least one microorganism and specifically at least one bacterium, also encompasses an isolate, a mutant, spores, enzymes or extracts thereof and/or a conditioned culture medium (growth medium) of the at least one bacterium and/or compounds (components/metabolites) secreted from the at least one bacterium.

The attribution of an identified microorganism to a microbial consortium of the invention was done by considering the multiple functions (i.e. multiple pathways) that each one of the identified microorganisms may have as well as the biological relationships between the microorganisms in a consortium. Each one of the microorganisms in the microbial consortium may be capable of modulating at least one process, at times at least two processes and even at times at least three or at least four process as detailed herein below. In addition, at times a metabolic product of one microorganism may be used as a substrate by another microorganism of the microbial consortium or alternatively by additional gut commensals or preventing the growth of certain gut commensals. This may increase the likelihood of intestinal colonization by the microbial consortium or promoting a desired activity of one or more microorganism.

In addition, attribution of an identified microorganism to a microbial consortium was done by considering the resulting combined functions of the consortium. For example, a specific process may be modulated by a single microorganism, or at times by at least two microorganisms, at least three microorganisms or even by at least four microorganisms in the microbial consortium. It was suggested that the combination of at least two microorganisms in the microbial consortium may achieve actions through several underlying, overlapping and complementary mechanisms (processes, pathways).

While some of the modulated processes may overlap between the two or more microorganisms, the consortium has a degree of diversity in order to obtain a broad and complementary effect.

In some embodiments, a microbial consortium comprising two or more microorganisms, at least one of the two or more microorganisms having one or more of the following features: (i) capable of modulating visceral hypersensitivity (ii) capable of modulating intraluminal gas balance, (iii) capable of modulating ENS, (iv) capable of modulating gut-brain axis, (v) capable of modulating GABA, (vi) capable of modulating gut-barrier (vii) capable of modulating pathways associated with low-grade inflammation, or (viii) combination thereof.

In some embodiments, a microbial consortium comprising two or more microorganisms, at least one of the two or more microorganisms having one or more of the following features: (i) capable of modulating fatty acid production, (ii) capable of modulating dietary compounds metabolism, (iii) capable of modulating GABA, (iv) capable of modulating detoxification activity, (v) capable of modulating an endopeptidase activity or (vi) combination thereof.

In accordance with some embodiments, at least one microorganism in the microbial consortium is capable of modulating one or more pathways related to (i) visceral hypersensitivity, (ii) intraluminal gas balance, (iii) ENS, (iv) gut-brain axis, (v) GABA levels (vi) gut-barrier enhancement, (vii) low-grade inflammation, (viii) fatty acid production, (ix) dietary compound transformation, (x) increased detoxification activity, (xi) inhibition of endopeptidase activity, or (xii) combination thereof.

The term modulate, modulation or modulating refers to an effect of altering a biological activity, i.e. a change, modification or the like that results in either activation/increased/enhanced activity or inhibition/decreased/reduction activity, for example of a physiological/cellular process and specifically, any of the processes of the invention as specified herein.

Hence, the microbial consortium comprising two or more microorganisms may be considered as either an activator or an inhibitor of a specific process (pathway).

An activator refers to at least one microorganism that induces, activates, stimulates, increases, facilitates, enhances activation, sensitizes or up regulates at least one physiological/cellular process.

The activation effect of the at least one microorganism may be associated with production of a substance as further described below. The substance can be either directly or indirectly associated with the process.

An inhibitor refers to at least one microorganism that inhibits, partially or totally blocks stimulation or activation, decreases, prevents, delays activation, inactivates, desensitizes, reduces or down regulates a physiological/cellular process.

The inhibitory effect of the at least one microorganism may be associated with reduction in the amount (content) of a substance as further described below.

It should be noted that when referring to at least one microorganism capable of producing a substance it is to be understood as increasing the amount (content) of the substance (by producing it) as compared to the amount that would have been produced in the absence of the at least one microorganism or as producing a substance that would not have been produced in the absence of the at least one microorganism.

It should be noted that when referring to at least one microorganism capable of decreasing/reducing (or the like) a substance it is to be understood as reducing or even decreasing the amount (content) of the substance as compared to it's amount as produced in the absence of the at least one microorganism.

Additionally or alternatively, the modulation by at least one microorganism in the microbial consortium also refers to an increase that is a result of the presence of the at least one microorganism in the microbial consortium which participates in a specific pathway and/or producing a specific product. The specific modulations are further described below.

As described herein, the present invention encompasses processes which are modulated endogenously in/by the microorganisms and/or processes which are modulated in the host by products being modulated (e.g. produced) in at least one microorganism in the microbial consortium and secreted or/and modified therefrom to a host. A physiological process as used herein encompasses physical and/or biological and/or chemical events in/by the microorganism and/or in the host.

Processes that are modulated endogenously in the microorganisms, i.e. microbial-endogenous processes are all collectively denoted herein as “microbial-component processes”. The microbial processes may take place in at least one specific type (species or strain) of microorganism (endogenous process). Such microbial component processes produce, for example, a microorganism's product such as a short chain fatty acid, that may be secreted from the microorganism to affect the host, including its use by the host in a host reaction. Processes that take place in the host by the use of a microorganism's components such as a product produced by at least one microorganism in the microbial consortium is denoted herein as “host-component processes”. As noted herein, serotonine production by a host cell can be modulated by the present of at least one microorganism.

In some embodiments, the microbial consortium is capable of modulating pathways related with the ENS. ENS (also known as intrinsic nervous system) is part of the autonomic nervous system (ANS) which governs the function of the gastrointestinal tract.

The ENS may act independently of the central nervous system (CNS) or can be influenced by the CNS.

In accordance with some embodiments, the microbial consortium is capable of modulating the ENS independently from the CNS.

In accordance with some other embodiments, ENS modulation by the microbial consortium is influenced by the CNS.

In some embodiments, the microbial consortium is capable of modulating processes/pathways related to the microbiome-gut-brain axis.

As used herein the expression microbiome-gut-brain axis refers to biochemical signaling pathways between the gastrointestinal tract (GI tract) and the ENS and/or the CNS which considers the role of microorganisms in the gut in the signaling pathway.

Modulation of the ENS and/or the microbiome-gut-brain axis encompasses modulation of metabolic/neuro-modulatory pathways that involve amino acids, including, inter alia, γ-aminobutyric acid (GABA).

In some embodiments, the microbial consortium is capable of modulating GABA production. In some embodiments, GABA modulation is mediated by at least one microorganism of the microbial consortia.

GABA is a major inhibitory neurotransmitter of the central nervous system (CNS). In accordance with the present disclosure, modulation of GABA encompasses a change in the amount/concentration of GABA in the GI tract in response to the microbial consortium.

A change in the amount/concentration of GABA includes one or more of the following: (i) increased GABA levels, (ii) increased activation of GABA receptors, (iii) increased the GABA production, (iv) inhibition of GABA degradation enzymes (e.g. host enzymes) or (v) combination thereof.

In some embodiments, the microbial consortium comprises at least one, at least two, at least three microorganisms which are capable of producing GABA. It is to be noted that enrichment of the host gut by such microorganisms results in increased amounts of GABA.

As noted above, the production of GABA may result in an increased amount as compared to the amount of GABA being produced in the absence of the microbial consortium. Hence, in some embodiments, the microbial consortium comprises at least one microorganism which is capable of producing GABA and hence the microbial consortium increases the production of GABA. The GABA produced by the microbial consortium is in accordance with some embodiments, secreted to the host gut.

Bacterial GABA can be produced in the gut via multiple pathways, some of which may be related pathways. For example, GABA can be produced by various GABA precursors, including, inter alia, putrescine or glutamate.

Therefore, in accordance with the present disclosure, modulation of GABA relates to the ability of at least one microorganism of the microbial consortiums of the invention to produce GABA as well as GABA precursors, precursors thereof (such as arginine) or co-factors (such as vitamin B6). As appreciated, the GABA precursors, the precursors thereof or the co-factors all can be used by at least one microorganism to produce GABA.

Hence, in some embodiments, the microbial consortium is capable of modulating one or more of (i) GABA, (ii) putrescine, (iii) glutamate, (iv) arginine, (v) vitamin B6 or (vi) combinations thereof.

In some other embodiments, the microbial consortium is capable of producing one or more of (i) putrescine, (ii) glutamate or (iii) combinations thereof.

In some further embodiments, the microbial consortium is capable of producing arginine.

In some further embodiments, the microbial consortium is capable of producing vitamin B6.

Surprisingly, as a unique feature of the present disclosure, it was found that at least one of the identified microorganisms is capable of producing valerate or isovalerate.

In some embodiments, the at least one or more microorganisms in the microbial consortium is capable of producing valerate, isovalerate or combination thereof.

In some embodiments, the at least one of the two or more microorganisms, at times at least two or more microorganisms in the microbial consortium are capable of producing valerate.

In some embodiments, the at least one of the two or more microorganisms in the microbial consortium is capable of producing one or more of (i) GABA, (ii) putrescine, (iii) glutamate, (iv) valerate, (v) isovalerate (vi) arginine, (vii) vitamin B6 or (viii) combinations thereof.

In some embodiments, the at least one of the two or more microorganisms in the microbial consortium is capable of producing one or more of (i) GABA, (ii) valerate, (iii) isovalerate or (iv) combinations thereof.

In some embodiments, the at least one of the two or more microorganisms in the microbial consortium is capable of producing one or more of (i) valerate, (ii) isovalerate or (iii) combinations thereof.

It was suggested that bacterial GABA could be secreted to the host gut cells where it can bind to host's GABA receptors. Without being bound by theory, it was suggested that valerate or isovalerate produced by the microbial consortia of the invention, can be secreted, similar to GABA, to the host gut cells and in turn bind to host's GABA receptors, optionally on the basis of structural similarity. It is of note that GABA, valerate or isovalerate may bind to host's GABA receptors in the GI tract and/or in the brain.

This suggests a unique microorganism-host pathway in which GABA receptors in the GI tract and/or the brain are activated and hence modulating ENS and/or CNS in a host.

Without being bound by theory, it was suggested that the microbial consortium is capable of producing GABA receptor agonists (e.g. GABA, valerate, isovalerate), which may be secreted to the host gut, to affect symptoms progression and specifically the symptoms of functional gastrointestinal conditions.

Modulation of GABA by the microbial consortium also encompasses inhibition of GABA degradation. It was suggested that the microbial consortium is capable of modulating GABA degradation enzymes.

In some embodiments, at least one microorganism in the microbial consortium is capable of modulating caproic acid (caproate). In some embodiments, the at least one of the two or more microorganisms in the microbial consortium is capable of producing caproate.

In some embodiments, the at least one of the two or more microorganisms in the microbial consortium is capable of producing one or more of (i) GABA, (ii) putrescine, (iii) glutamate, (iv) valerate, (v) isovalerate (vi) arginine, (vii) vitamin B6, (viii) caproate or (ix) combinations thereof.

The microbial consortium is capable of modulating neurotransmitters, including neurotransmitters that are synthesized from amino acids. In some embodiments, the microbial consortium is capable of modulating serotonin levels. In some embodiments, the microbial consortium is capable of increasing serotonin levels.

As shown herein below, cells incubated with cultured growth medium of at least one microorganism are characterized by increased secretion of serotonin from the cells. Without being bound by theory, it was suggested that cultured growth medium of the at least one microorganism that may produce metabolites and/or comprise components from the microorganism, being secreted to the growth medium, may affect processes in cells incubated with the growth medium, including, inter alia, production of serotonin. Hence, it was suggested that the microbial consortium may modulate a microbial process that results in the production of serotonin by a host cell and optional secretion.

In some embodiments, the microbial consortium is capable of modulating lactate production. Accordingly, the microbial consortium may modulate a microbial process that results in the production of lactate. As detailed herein, lactate produced by at least one microorganism in the microbial consortium and may by secreted to the host (possibly host gut).

It was suggested that lactate, and/or acetate, and/or GABA which is produced by at least one microorganism in the microbial consortium may by secreted to the host and modulate host related processes, including, inter alia, affecting serotonin levels in the gut, specifically increase serotonin levels.

In some embodiments, the microbial consortium is capable of modulating pathways related with intraluminal gas balance.

Intraluminal gas may be from a source of air swallowing or accumulation of amounts of microbial fermentation gases in the lumen of the gastrointestinal system, including, inter alia, N₂, O₂, CO₂, H₂, CH₄, H₂S or CO.

It was suggested that microorganisms forming part of the microbial consortium are capable of utilizing intraluminal gas. Thus, by enriching the gut microbiome with the microbial consortium of the present invention, intraluminal gas, such as hydrogen gas is reduced in the gut. In other words, the microorganisms (at least one or more) forming part of the microbial consortium are capable of lowering/reducing the total gas level in the gut.

Utilization of intraluminal gas by the microorganisms of the invention may produce host beneficial metabolites including, inter alia, short chain fatty acids (SCFAs). Additionally or alternatively, enriching the host gut with microorganisms that utilize intraluminal gas may compete with other gas-consuming microorganisms in order to reduce or prevent undesirable production of non-beneficial (non favorable) products, including, inter alia, methane and/or H₂S.

In some embodiments, at least one of the two or more microorganisms in the microbial consortium is capable of modulating intraluminal gas consumption. Specifically, the microbial consortium is capable of lowering (decreasing) the amount of luminal gas in the gut.

In some embodiments, at least one of the two or more microorganisms in the microbial consortium is capable of modulating carbon monoxide consumption, carbon dioxide consumption or combination thereof. Specifically, the microbial consortium is capable of lowering the amount of carbon dioxide and carbon monoxide in the gut.

In some embodiments, at least one of the two or more microorganisms in the microbial consortium is capable of modulating hydrogen consumption. Specifically, the microbial consortium is capable of lowering the amount of hydrogen in the gut.

In some embodiments, at least one of the two or more microorganisms in the microbial consortium is capable of lowering the amount of (i) carbon dioxide, (ii) carbon monoxide, (iii) hydrogen or (iv) any combination thereof.

In some embodiments, the microorganisms in the microbial consortium use hydrogen in the gut for reduction processes.

It was suggested that the microbial consortium is capable of consuming intraluminal gas (i.e. reducing gases level in the gut) via production of various metabolites.

It was suggested that at least one or more microorganisms utilize luminal gas such as hydrogen gas to produce SCFAs which are in turn may be secreted to the gut.

In some embodiments, the microbial consortium is capable of producing at least one SCFA, optionally by intraluminal gas consumption.

In some embodiments, at least one of the two or more microorganisms is capable of producing at least one of acetate, or propionate by using the intraluminal gas.

In some embodiments, at least one of the two or more microorganisms is capable of producing acetate, propionate or combination thereof.

In some embodiments, at least one of the two or more microorganisms is capable of producing acetate.

In some other embodiments, at least one of the two or more microorganisms is capable of producing propionate.

Additionally, it was suggested that at least one of the two or more microorganisms is capable of producing ammonia by using the intraluminal hydrogen gas.

Surprisingly, it was found that the microbial consortium is capable of modulating nitrogen compounds.

As used herein nitrogen compounds (or nitrogen containing compounds) refer to a molecule containing at least one nitrogen atom.

In some embodiments, at least one of the two or more microorganisms is capable of modulating the nitrate-nitrite reduction pathway.

In some embodiments, at least one of the two or more microorganisms is capable of modulating nitrate to be reduced to nitrite. In some embodiments, at least one of the two or more microorganisms is capable of modulating nitrite to be reduced to ammonia. Ammonia in turn is secreted to the host gut.

In some embodiments, modulating hydrogen consumption comprises one or more of (i) modulating SCFAs (ii) modulating nitrate-nitrite reduction pathway or (iii) combination thereof.

In some embodiments, modulating intraluminal gas consumption comprises one or more of (i) producing acetate, (ii) producing propionate, (iii) producing ammonia, (iv) producing nitrite or (v) combination thereof.

As described herein, decreasing (lowering) the level of intraluminal hydrogen gas using the microorganisms of the invention, decreases the available hydrogen gas which is typically required for reduction processes in the gut and hence inhibiting reduction reactions and their products.

Non-limiting examples of such products include methane or hydrogen sulfide.

In some embodiments, modulating intraluminal gas consumption comprises decreasing and at times inhibiting methanogenesis.

Methanogenesis (also known as bio-methanation) refers to formation of methane.

In some embodiments, modulating hydrogen consumption comprises decreasing methane levels in the gut. Hence, in accordance with the present disclosure, the microbial consortium decreases methane levels.

In some embodiments, modulating intraluminal gas consumption comprises decreasing sulphate reduction. In some embodiments, modulating intraluminal gas consumption comprises inhibiting sulphate reduction.

In some embodiments, modulating hydrogen consumption comprises decreasing hydrogen sulfide levels in the gut. Hence, in accordance with the present disclosure, the microbial consortium decreases hydrogen sulfide levels.

In some embodiments, modulating intraluminal gas consumption comprises lowering gas levels in the gut (i.e. intraluminal gas). In some embodiments, modulating intraluminal gas consumption comprises lowering hydrogen gas levels in the gut (i.e. intraluminal gas). In some embodiments, modulating intraluminal gas consumption comprises inhibiting reduction reactions in the gut.

In some embodiments, modulating intraluminal gas consumption comprises one or more of (i) production of SCFAs (ii) modulating nitrate-nitrite reduction pathway, (iii) decreasing methanogenesis, (iv) inhibiting sulfate reduction or (v) combination thereof.

In some embodiments, modulating intraluminal gas consumption comprises one or more of (i) producing acetate, (ii) producing propionate, (iii) producing ammonia, (iv) producing nitrite, (v) decreasing methane levels, (vi) decreasing hydrogen sulfide levels or (vii) combination thereof.

In some embodiments, modulating hydrogen gas consumption comprises one or more of (i) producing acetate, (ii) producing propionate, (iii) producing ammonia, (iv) producing nitrite, (v) decreasing methane levels, (vi) decreasing hydrogen sulfide levels or (vii) combination thereof.

In some embodiments, modulating carbon monoxide (CO) gas consumption comprises producing acetate. In some embodiments, modulating carbon dioxide (CO₂) gas consumption comprises producing acetate.

In addition to production of at least a SCFA by utilizing intraluminal hydrogen gas, SCFAs levels can be modulated via various pathways.

Hence, the microbial consortium is capable of modulating at least one SCFA.

In addition to modulation of SCFAs, e.g. production of SCFAs, the microbial consortium is capable of modulating additional fatty acids including, inter alia, medium-chain fatty acids (MCFAs), long-chain fatty acids (LCFAs) or very-long chain fatty acids (VLCFAs). Hence, in some embodiments, the microbial consortium is capable of modulating at least one of SCFA, MCFA, LCFA, VLCFA or combination thereof. In some embodiments, the microbial consortium is capable of producing at least one of SCFA, MCFA, LCFA, VLCFA or combination thereof.

In accordance with such embodiments, the at least one of the two or more microorganisms is capable of modulating at least one SCFA, specifically acetate, butyrate, propionate, valerate, isovalerate or combination thereof.

In accordance with such embodiments, the at least one of the two or more microorganisms is capable of producing one or more of acetate, butyrate, propionate, valerate, isovalerate or combination thereof.

In accordance with such embodiments, the at least one of the two or more microorganisms is capable of modulating at least one MCFA. In some embodiments, the at least one of the two or more microorganisms is capable of producing at least one of caproate, caprylic acid, capric acid, lauric acid or combination thereof. In some embodiments, the at least one of the two or more microorganisms is capable of producing caproate.

In accordance with such embodiments, the at least one of the two or more microorganisms is capable of modulating at least one LCFA.

In some embodiments, the at least one of the two or more microorganisms is capable of producing at least one LCFA.

In some embodiments, the microbial consortium is capable of producing an unsaturated fatty acid.

In some embodiments, the microbial consortium is capable of producing linoleic acid.

In some embodiments, the microbial consortium is capable of producing one or more of acetate, butyrate, propionate, valerate, isovalerate, caproate, linoleic acid or combination thereof.

In some embodiments, the microbial consortium is capable of producing one or more of acetate, butyrate, propionate, valerate, isovalerate or combination thereof.

In some embodiments, the microbial consortium is capable of producing one or more of acetate, propionate or combination thereof.

In some embodiments, the microbial consortium is capable of producing one or more of acetate, GABA or combination thereof.

Without being bound, it was suggested that the fatty acids produced by the microbial consortium (in an exemplary microbial-component process) provide beneficial effects on the host by modulating host pathways (in an exemplary host-component process). Such host processes may relate for example to pain. It was further suggested that the fatty acids produced by the microbial consortium bind to peroxisome proliferator-activated receptors (PPARs).

Hence, in some embodiments, the microbial consortium is capable of producing PPAR ligands and specifically PPARγ ligands.

Non-limiting examples of PPARγ ligands include fatty acids, such as butyrate and propionate.

The microbial consortium is also capable of modulating dietary compounds.

Dietary compound as used herein refers to compounds found in food and/or beverages, for example, of plant origin. For example, dietary compounds include isoflavonoids.

In accordance with some embodiments, the microbial consortium is capable of modulating at least one isoflavonoid.

In accordance with some embodiments, the microbial consortium is capable of metabolizing at least one isoflavonoid.

Isoflavonoids are a class of flavonoid phenolic compounds, at times referred to a phytoestrogen and include, for example, isoflavones, isoflavonones, isoflavans, or pterocarpans.

In some embodiments, the microbial consortium is capable of metabolizing at least one isoflavone. In some embodiments, the microbial consortium is capable of producing daidzein and genistein. In some embodiments, the at least one isoflavone is metabolized by the microbial consortium to produce daidzein and genistein.

Without being bound by theory, it was suggested that at least one microorganism in the microbial consortium is capable of metabolizing at least one isoflavone, a product of which, such as genistein and/or daidzein, may modulate a host component.

Non-limiting examples of a host component that may be modulated by an isoflavone metabolism product is prostaglandin, such as Prostaglandin E2 or nitric oxide.

Specifically, it was suggested that at least one microorganism in the microbial consortium produces genistein and/or daidzein which act as a COX-2-inhibitor to inhibit Prostaglandin E2 production.

In some embodiments, the microbial consortium is capable of modulating vitamin B12.

The microbial consortium is also capable of modulating host's enzymatic pathways, by the activity of microbial proteins that bind to the host's enzyme. In some embodiments, the at least one of the two or more microorganisms in the microbial consortium is capable of modulating the activity of an endopeptidase.

Endopeptidase (also referred to as endoproteinase) are a group of proteolytic peptidases that break peptide bonds of nonterminal amino acids (i.e. within the molecule). It was suggested that microbial proteins, including, inter alia, serpins, are capable of inhibiting the activity of host's endopeptidase, optionally a serine protease.

In some embodiments, the at least one of the two or more microorganisms in the microbial consortium is capable of modulating a serine protease. In some embodiments, the at least one of the two or more microorganisms in the microbial consortium is capable of decreasing activity of at least one serine protease. In some embodiments, the at least one of the two or more microorganisms in the microbial consortium is capable of inhibiting activity of at least one serine protease.

In some embodiments, the microbial consortium is capable of modulating activities related to intestinal mucosal barrier (also denoted as intestinal barrier or gut-barrier) and specifically to the integrity and the functionality of the intestinal mucosa. As appreciated by those versed in the art, the intestinal mucosa ensures adequate surroundings of the intestine and provides a separation between the body and the gut and hence prevents uncontrolled translocation of luminal contents into the body. Dysfunction of the intestinal mucosal barrier may be associated with a variety of pathologies, including but not limited to gastrointestinal disorders.

In some embodiments, the microbial consortium assists in maintaining activity of the gut-barrier, i.e. a functional gut-barrier.

The activity/integrity/functionality of the gut-barrier is associated with a variety of components and pathways.

It was surprisingly found that commensal microorganisms and specifically at least one microorganism in the microbial consortium is capable of modulating the level of hormones. Such hormones are dentoed as steroid hormone.

In some embodiments, the at least one steroid hormone is at least one of a corticosteroids, glucocorticoids, mineralocorticoids, a sex steroid or a combination thereof.

In some embodiments, the at least one steroid hormone is at least one of a corticosteroids.

In some embodiments, the at least one steroid hormone is at least one of a sex steroid.

In some embodiments, the at least one steroid hormone is at least one of progesterone, 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), and 5α-dihydrodeoxycorticosterone (5α-DHDOC) or any combination thereof.

In some embodiments, the at least one steroid hormone is at least one of progesterone, testosterone, 17α-hydroxyprogesterone and androstenedione.

In some embodiments, the at least one steroid hormone is progesterone, 17α-hydroxyprogesterone or combinations thereof.

In some other embodiments, the at least one steroid hormone is progesterone.

In some embodiments, the microbial consortium is capable of increasing the levels of hormones. In some embodiments, the microbial consortium is capable of increasing the levels of estrogen and/or progesterone. In some embodiments, the microbial consortium is capable of producing progesterone.

Without being bound by theory, it was suggested that the increased production of at least one hormone, including, inter alia, progesterone may be mediated by the activity of 3β-HSD.

In some embodiments, enhancement of gut-barrier is associated with at least one of (i) bile acids, (ii) SCFAs, (iii) putrescine, (iv) hormones, (v) isoflavonoids, (vi) GABA or (vii) any combinations thereof.

In some embodiments, enhancement of gut-barrier is associated with at least one of (i) bile acids, (ii) SCFAs, (iii) putrescine, (iv) estrogen, (v) progesterone, (vi) isoflavonoids, (vii) GABA or (viii) any combinations thereof.

In some embodiments, enhancement of gut-barrier is associated with at least one of (i) bile acid, (ii) butyrate, (iii) acetate, (iv) putrescine, (v) hormones, (vi) isoflavonoids, (vii) propionate, (viii) GABA or (ix) any combinations thereof.

In some embodiments, enhancement of gut-barrier is associated with at least one of (i) secondary bile acid production, (ii) butyrate production, (iii) acetate production, (iv) putrescine production, (v) hormones production, (vi) isoflavonoids metabolism, (vii) propionate production, (viii) GABA production or (ix) any combinations thereof.

In some embodiments, the microbial consortium is capable of modulating the metabolism of environmental pollutants. In some embodiments, the environmental pollutant is an air pollution, water pollution or food pollution. In some embodiments, the environmental pollution is an air pollution, such as CO. In some embodiments, the environmental pollution is a metal such as arsenic.

In some embodiments, the microbial consortium is capable of modulating pathways related to low grade inflammation.

Low-grade inflammation is associated with a low-level of inflammation throughout the body, as indicated by a small rise in immune system markers found in blood or tissue.

In some embodiments, the microbial consortium is capable of attenuating/inhibiting/reducing activity of various pathways collectively associated with low-grade inflammation. In some embodiments, reducing low grade inflammation is associated with any one of (i) iron sequestering, (ii) nitric oxide, (iii) bile acid, (iv) detoxification of environmental pollutants (v) SCFAs or (vi) any combinations thereof.

In some embodiments, reducing low grade inflammation is associated with any one of (i) iron sequestering, (ii) degrading nitric oxide, (iii) production of secondary bile acids, (iv) detoxification of environmental pollutants (v) production of SCFAs or (vi) any combinations thereof.

In some other embodiments, reducing low grade inflammation is associated with any one of (i) iron sequestering, (ii) reducing nitric oxide, (iii) production of secondary bile acids, (iv) detoxification of environmental pollutants, (v) production of acetate, (vi) production of butyrate, (vii) production of propionate, (viii) production of valerate or (ix) any combinations thereof.

In some embodiments, the microbial consortium is capable of modulating iron sequestering. It was suggested that modulating iron sequestering, specifically increasing iron sequestering may assist in preventing damages related with oxidative stress.

It was surprisingly found that at least one microorganism in the microbial consortium is capable of modulating the level of nitric oxide. It was suggested that the at least one microorganism is capable of degrading nitric oxide.

In some embodiments the microbial consortium is capable of decreasing nitric oxide levels.

In some embodiments, the microbial consortium is capable of modulating bile acid.

It is of note that when reference made to microbial consortium modulating bile acid relates to at least one microorganism in the microbial consortium being capable of modulating host' secondary bile acid.

In accordance with the present disclosure, those microorganism(s) that modulate secondary bile acid are in fact microorganism(s) that are capable of transforming/converting host's primary bile acids to secondary bile acids. Hence, the secondary bile acid in the context of the invention refers to a host product following modification by microorganism's activity, specifically those microorganisms in the microbial consortium

Conversion of host's primary bile acids to secondary bile acids may be done by any microorganisms' enzyme capable of this reaction. For example, an enzyme may include, inter alia, 7-alpha-dehydroxylase.

In some embodiments, at least one of the two or more microorganisms in the microbial consortium modulates secondary bile acid. As noted above, at least one microorganism in the microbial consortium is capable of affecting the synthesis of secondary bile acids in the host gut hence modulating the secondary bile acids level (content/amount) in a host.

In accordance with some other embodiments, at least one of the two or more microorganisms in the microbial consortium is capable of producing secondary bile acids in the host.

In some embodiments, the secondary bile acid comprises deoxycholic acid (DOC), isoallo-lithocholic acid (isoalloLCA) or any combinations thereof.

In some embodiments, at least one of the two or more microorganisms in the microbial consortium is capable of producing DOC.

In some embodiments, at least one of the two or more microorganisms in the microbial consortium is capable of producing isoalloLCA.

In some embodiments, the microbial consortium is capable of modulating regulatory T cells (Tregs). It was suggested that the microbial consortium increased the amount/number of Tregs. Hence, in the context of the present disclosure, the microbial consortium is capable of Tregs differentiation. Tregs are a subpopulation of T cells that modulate the immune system, maintain tolerance to self-antigens, and prevent autoimmune disease.

In some embodiments, the microbial consortium is capable of modulating at least one pathway related to visceral hypersensitivity.

Visceral hypersensitivity (also known as visceral hyperalgesia) as used herein refers to existence of higher sensation of abdominal sensitivity including, inter alia, bloating or/and pain within the inner organs (viscera) at a level that is more intense than normal. Visceral hypersensitivity may be associated with heightened sensitivity to gut stimulation, including, peripheral sensitization, central sensitization, aberrant central processing, genetic, psychological and abnormalities within the stress responsive systems.

It should be noted that modulating visceral hypersensitivity may be a result of various pathways, optionally interkingdom pathways. In some embodiments, modulating of intraluminal gas and specifically lowering the level/amount of intraluminal gas, positively affect visceral hypersensitivity.

In accordance with some embodiments, the microbial consortium comprising two or more microorganisms, at least one of the two or more microorganisms, at times at least two of the two or more microorganism is capable of modulating one or more of (i) putrescine, (ii) glutamate, (iii) vitamin B6, (iv) vitamin B12, (v) arginine, (vi) GABA, (vii) fatty acids, (viii) gut-barrier, (ix) serine protease activity, (x) lactate, (xi) iron sequestering, (xii) bile acid, (xiii) pollutants detoxification, (xiv) methanogenesis, (xv) sulphate reduction, (xvi) Treg, (xvii) nitrogen compound, (xviii) nitric oxide, (xix) hormones, (xx) isoflavonoids, (xxi) serotonin or (xxii) any combinations thereof.

In accordance with some embodiments, the microbial consortium comprising two or more microorganisms, at least one of the two or more microorganisms, at times at least two of the two or more microorganism is capable of modulating one or more of (i) putrescine, (ii) glutamate, (iii) vitamin B6, (iv) vitamin B12, (v) arginine, (vi) GABA, (vii) SCFAs, (viii) MCFAs (ix) LCFAs (x) gut-barrier, (xi) serine protease activity, (xii) lactate, (xiii) iron sequestering, (xiv) bile acid, (xv) pollutants detoxification, (xvi) methanogenesis, (xvii) sulphate reduction, (xviii) Treg, (xix) ammonia, (xx) nitrate, (xxi) nitric oxide, (xxii) estrogen, (xxiii) progesterone (xxiv) isoflavonoids (xxv) serotonin or (xxvi) any combinations thereof.

In accordance with some embodiments, the microbial consortium comprising two or more microorganisms, at least one of the two or more microorganisms, at times at least two of the two or more microorganism is capable of modulating one or more of (i) putrescine, (ii) glutamate, (iii) vitamin B6, (iv) vitamin B12, (v) arginine, (vi) GABA, (vii) acetate, (viii) butyrate, (ix) propionate, (x) valerate, (xi) isovalerate, (xii) caproate, (xiii) linoleic acid, (xiv) serine protease activity, (xv) lactate, (xvi) iron sequestering, (xvii) secondary bile acid, (xviii) pollutants detoxification, (xix) methanogenesis, (xx) sulphate reduction, (xxi) Treg, (xxii) ammonia, (xxiii) nitrate, (xxiv) nitric oxide, (xxv) estrogen, (xxvi) progesterone (xxvii) isoflavonoids (xxviii) serotonin or (xxix) any combinations thereof.

In accordance with some embodiments, the microbial consortium comprising two or more microorganisms, at least one of the two or more microorganisms, at times at least two of the two or more microorganism is capable of having at least one, at least two, at least three, at least four, at least five, at least six, at least seven, at least eight, at least nine, at least ten, at least elven, at least twelve, at least thirteen or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with some embodiments, the microbial consortium comprising two or more microorganisms, at least one of the two or more microorganisms, at times at least two of the two or more microorganism is capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease (xv) lactate production, (xvi) increasing iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with some embodiments, the microbial consortium comprising two or more microorganisms, at least one of the two or more microorganisms, at times at least two of the two or more microorganism is capable of having at least eight of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease (xv) lactate production, (xvi) increasing iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In accordance with some embodiments, the microbial consortium comprising two or more microorganisms, at least one of the two or more microorganisms is capable of having one or more of (i) putrescine production, (ii) glutamate production, (iii) vitamin B6 production, (iv) vitamin B12 production, (v) GABA production, (vi) acetate production, (vii) butyrate production, (viii) propionate production, (ix) valerate production, (x) isovalerate production, (xi) caproate production, (xii) linoleic acid production, (xiii) decreasing activity of at least one serine protease (xiv) lactate production, (xv) secondary bile acid production, (xvi) decreasing of methanogenesis, (xvii) deceasing sulphate reduction process (xviii) ammonia production, (xix) nitrite production, or (xx) any combinations thereof.

In accordance with some embodiments, the microbial consortium comprising two or more microorganisms, at least one of the two or more microorganisms, at times two of the two or more microorganisms is capable of modulating one or more of (i) GABA (ii) SCFAs, (iii) nitrogen compounds, (iv) lactate, (v) methanogenesis, (vi) sulphate reduction, (vii) linoleic acid, (viii) prostaglandin E2, (ix) vitamins B12, (x) serine protease activity or (xi) any combinations thereof.

In accordance with some embodiments, the microbial consortium comprising two or more microorganisms, at least one of the two or more microorganisms, at times two of the two or more microorganisms is capable of modulating one or more of (i) GABA, (ii) putrescine, (iii) glutamate, (iv) valerate, (v) isovalerate (vi) arginine, (vii) vitamin B6, (viii) caproate, (ix) acetate, (x) butyrate, (xi) propionate, (xii) ammonia, (xiii) lactate, (xiv) methanogenesis, (xv) sulphate reduction, (xvi) serine protease activity, (xvii) vitamin B12, (xviii) linoleic acid, (xix) prostaglandin E2, (xx) isoflavonoid (xxi) any combinations thereof.

In accordance with some embodiments, the microbial consortium comprising two or more microorganisms, at least one of the two or more microorganisms, at times two of the two or more microorganisms is capable of having at least five of (i) vitamin B12 production, (ii) GABA production (iii) valerate production, (iv) isovalerate production, (v) decreasing activity of at least one serine protease, (vi) increasing iron sequestering, (vii) caproate production, (viii) pollutants detoxification, (ix) linoleic acid production (x) acetate production, (xi) butyrate production, (xii) propionate production, (xiii) decreasing methanogenesis, (xiv) ammonia production, (xv) nitrite production, (xvi) nitric oxide degradation, (xvii) isoflavonoids metabolism (xviii) decreasing sulphate reduction or (xix) any combinations thereof.

In accordance with some embodiments, the microbial consortium comprising two or more microorganisms, at least one of the two or more microorganisms, at times two of the two or more microorganisms is capable of having at least five of (i) vitamin B12 production, (ii) GABA production (iii) valerate production, (iv) increasing iron sequestering, (v) caproate production, (vi), linoleic acid production (vii) acetate production, (vii) butyrate production, (ix) propionate production, (x) decreasing methanogenesis, (xi) ammonia production, (xii) nitrite production, or (xiii) any combinations thereof.

In accordance with some embodiments, the microbial consortium comprising two or more microorganisms, at least one of the two or more microorganisms, at times two of the two or more microorganisms is capable of at least seven of (i) vitamin B12 production, (ii) GABA production (iii) valerate production, (iv) increasing iron sequestering, (v) caproate production, (vi), linoleic acid production (vii) acetate production, (vii) butyrate production, (ix) propionate production, (x) decreasing methanogenesis, (xi) ammonia production, (xii) nitrite production, or (xiii) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof,

wherein at least one of the two or more microorganisms is capable of having at least one of, at times at least two of, at times at least three of, at least four of, at times at least five of, at times six of (i) vitamin B12 production, (ii) GABA production, (iii) valerate production, (iv) isovalerate production, (v) decreasing activity of at least one serine protease, (vi) increasing iron sequestering, (vii) caproate production, (viii) pollutants detoxification, (ix) linoleic acid production, (x) acetate production, (xi) butyrate production, (xii) propionate production, (xiii) decreasing methanogenesis, (xiv) ammonia production, (xv) nitrite production, (xvi) nitric oxide degradation, (xvii) isoflavonoids metabolism or (xviii) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having at least one of, at least two of, at least three of, at least four of, at least five of, at times at least six of, at times at least seven of (i) vitamin B12 production, (ii) GABA production (iii) valerate production, (iv) isovalerate production, (v) decreasing activity of at least one serine protease, (vi) increasing iron sequestering, (vii) caproate production, (viii) pollutants detoxification, (ix) linoleic acid production (x) acetate production, (xi) butyrate production, (xii) propionate production, (xiii) decreasing methanogenesis, (xiv) ammonia production, (xv) nitrite production, (xvi) nitric oxide degradation, (xvii) isoflavonoids metabolism (xviii) decreasing sulphate reduction or (ixx) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide reduction, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having one or more of (i) GABA production, (ii) valerate production, (iii) isovalerate production, (iv) acetate production, (v) butyrate production, (vi) propionate production or (vii) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having one or more of (i) acetate production, (ii) butyrate production, (iii) propionate production, (iv) decreasing methanogenesis or (v) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide reduction, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having one or more of (i) acetate production, (ii) butyrate production, (iii) propionate production, or (iv) any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having one or more of valerate, isovalerate, or any combinations thereof.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of reducing nitrate to nitrite

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of producing progesterone.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of reducing nitric oxide.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of decreasing at least one of methanogenesis, sulfate reduction or combination thereof. In some embodiments, the at least one of the two or more microorganisms is capable of inhibiting methanogenesis.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of metabolizing at least one isoflavonoid.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of decreasing activity of at least one serine protease. In some embodiments, the at least one of the two or more microorganisms is capable of inhibiting serine protease activity.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of producing vitamin B6.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of producing vitamin B12.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of pollutants detoxification.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of inducing secretion of serotonin.

In accordance with some embodiments, the microbial consortium comprising two or more microorganisms, the microbial consortium is capable of having one or more of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof, wherein at least one of the two or more microorganisms is capable of having at least one, at times at least two, at times at least three, at times at least four, or more of (i) producing valerate, (ii) producing isovalerate, (iii) reducing nitrate to nitrite, (iv) producing progesterone, (v) reducing nitric oxide, (vi) decreasing methanogenesis, (vii) metabolizing at least one isoflavonoid, (viii) decreasing activity of at least one serine protease, (ix) producing vitamin B6, (x) producing vitamin B12, (xi) pollutants detoxification or (xii) any combinations thereof.

In accordance with some embodiments, the microbial consortium comprising two or more microorganisms, at least one of the two or more microorganisms is capable of having one or more of (i) vitamin B12 production, (ii) valerate production, (iii) isovalerate production, (iv) iron sequestering, (v) caproate production (vi) acetate production, (vii) butyrate production, (viii) propionate production, (ix) decreasing methanogenesis, (x) decrease sulphate reduction, (xi) ammonia production, (xii) nitrite production, (xiii) linoleic acid production or (xiv) any combinations thereof.

In accordance with some embodiments, the microbial consortium comprising two or more microorganisms, at least one of the two or more microorganisms is capable of having one or more of (i) vitamin B12 production, (ii) valerate production, (iii) isovalerate production, (iv) iron sequestering, (v) caproate production (vi) acetate production, (vii) butyrate production, (viii) propionate production, (ix) decrease in methane level, (x) decreasing sulphate reduction, (xi) ammonia production, (xii) nitrite production, (xiii) linoleic acid production or (xiv) any combinations thereof.

In accordance with some embodiments, the microbial consortium comprising two or more microorganisms, at least one of the two or more microorganisms is capable of having one or more of (i) valerate production, (ii) isovalerate production, (iii) acetate production, (iv) butyrate production, (v) propionate production, or (vi) any combinations thereof.

In accordance with some embodiments, the microbial consortium comprising two or more microorganisms, at least one of the two or more microorganisms is capable of having one or more of (i) valerate production (ii) acetate production, (iii) butyrate production, (iv) propionate production, or (v) any combinations thereof.

In accordance with some embodiments, the microbial consortium comprising two or more microorganisms, at least one of the two or more microorganisms is capable of having one or more of (i) isovalerate production (ii) acetate production, (iii) butyrate production, (iv) propionate production, or (v) any combinations thereof.

In accordance with some embodiments, the microbial consortium comprising two or more microorganisms, at least one of the two or more microorganisms is capable of having one or more of (i) acetate production, (ii) butyrate production, (iii) propionate production, or (iv) any combinations thereof.

In accordance with some embodiments, the microbial consortium comprising two or more microorganisms, at least one of the two or more microorganisms is capable of having one or more of (i) vitamin B12 production, (ii) valerate production, (iii) isovalerate production, (iv) nitrite production, (v) decrease in methane level, (v) decrease sulphate reduction, or (vi) any combinations thereof.

In accordance with some embodiments, the microbial consortium comprising two or more microorganisms, at least one of the two or more microorganisms is capable of having one or more of (i) valerate production, (ii) isovalerate production, (iii) nitrite production, (iv) decrease in methane level, (v) decrease sulphate reduction, or (vi) any combinations thereof.

In accordance with some embodiments, the microbial consortium comprising two or more microorganisms, at least one of the two or more microorganisms is capable of having one or more of (i) putrescine production, (ii) lactate production, (iii) iron sequestering, (iv) gut-barrier enhancement or (vi) any combinations thereof.

In accordance with some embodiments, the microbial consortium comprising two or more microorganisms, at least one of the two or more microorganisms is capable of having one or more of (i) putrescine production, (ii) glutamate production, (iii) vitamin B6 production, (iv) arginine production, (v) valerate production, (vi) isovalerate production, (vii) lactate production, (viii) iron sequestering, (ix) gut-barrier enhancement (x) ammonia production, (xi) nitrite production, (xii) caproate production or (xiii) any combinations thereof.

In accordance with some embodiments, the microbial consortium comprising two or more microorganisms, at least one of the two or more microorganisms is capable of having one or more of (i) valerate production, (ii) isovalerate production, (iii) nitrite production, (iv) vitamin B6 production, or (v) any combinations thereof.

In accordance with some embodiments, the microbial consortium comprising two or more microorganisms, at least one of the two or more microorganisms is capable of having one or more of (i) vitamin B6 production, (ii) GABA production, (iii) decreasing activity of at least one serine protease, (iv) lactate production, (v) acetate production, (vi) gut-barrier enhancement, (vii) serotonin production or (viii) any combinations thereof.

In accordance with some embodiments, the microbial consortium comprising two or more microorganisms, at least one of the two or more microorganisms is capable of having one or more of (i) GABA production, (ii) acetate production, (iii) serotonin production or (iv) any combinations thereof.

In accordance with some embodiments, the microbial consortium comprising two or more microorganisms, at least one of the two or more microorganisms is capable of having one or more of (i) GABA production, (ii) acetate production, or (iii) any combinations thereof.

In accordance with some embodiments, the microbial consortium comprising two or more microorganisms, at least one of the two or more microorganisms is capable of having one or more of (i) GABA production, (ii) serotonin production, or (iii) any combinations thereof.

In accordance with some embodiments, the microbial consortium comprising two or more microorganisms, at least one of the two or more microorganisms is capable of having one or more of (i) decreasing activity of at least one serine protease, (ii) acetate production or (iii) any combinations thereof.

In accordance with some embodiments, the microbial consortium comprising two or more microorganisms, at least one of the two or more microorganisms is capable of having one or more of (i) putrescine production, (ii) acetate production (iii) butyrate production, (iv) propionate production, (v) decreasing methanogenesis, (vi) decreasing sulphate reduction or (vii) any combinations thereof.

In accordance with some embodiments, the microbial consortium comprising two or more microorganisms, at least one of the two or more microorganisms is capable of having one or more of (i) putrescine production, (ii) acetate production (iii) butyrate production, (iv) propionate production, (v) decrease in methane level, (vi) decrease in sulphate reduction or (vii) any combinations thereof.

In accordance with some embodiments, the microbial consortium comprising two or more microorganisms, at least one of the two or more microorganisms is capable of having one or more of (i) acetate production, (ii) propionate production, or (iii) any combinations thereof.

In accordance with some embodiments, the microbial consortium comprising two or more microorganisms, at least one of the two or more microorganisms is capable of having one or more of (i) decreasing methanogenesis, (ii) decrease in sulphate reduction or (iii) any combinations thereof.

In accordance with some embodiments, the microbial consortium comprising two or more microorganisms, at least one of the two or more microorganisms is capable of having one or more of (i) vitamin B6 production, (ii) valerate production, (iii) isovalerate production, (iv) acetate production, (v) decreasing methanogenesis, (vi) decreasing sulphate reduction, (vii) Treg induction, or (viii) any combinations thereof.

In accordance with some embodiments, the microbial consortium comprising two or more microorganisms, at least one of the two or more microorganisms is capable of having one or more of (i) vitamin B6 production, (ii) valerate production, (iii) isovalerate production, (iv) acetate production, (v) decrease in methane level (vi) decreasing sulphate reduction, (vii) Treg induction, or (viii) any combinations thereof.

In accordance with some embodiments, the microbial consortium comprising two or more microorganisms, at least one of the two or more microorganisms is capable of having one or more of (i) vitamin B6 production, (ii) valerate production, (iii) acetate production, (iv) decreasing methanogenesis, (v) decreasing sulphate reduction, (vi) Treg induction, or (vii) any combinations thereof.

In accordance with some embodiments, the microbial consortium comprising two or more microorganisms, at least one of the two or more microorganisms is capable of having one or more of (i) vitamin B6 production, (ii) valerate production, (iii) acetate production, (iv) decrease in methane level, (v) decreasing sulphate reduction, (vi) Treg induction, or (vii) any combinations thereof.

In accordance with some embodiments, the microbial consortium comprising two or more microorganisms, at least one of the two or more microorganisms is capable of having one or more of (i) vitamin B6 production, (ii) valerate production, (iii) isovalerate production, (iv) decreasing methanogenesis, (vi) decreasing sulphate reduction, or (vii) any combinations thereof.

In accordance with some embodiments, the microbial consortium comprising two or more microorganisms, at least one of the two or more microorganisms is capable of having one or more of (i) putrescine production, (ii) vitamin B6 production, (iii) GABA production, (iv) secondary bile acid production, (v) pollutants detoxification, (vi) acetate production, (vii) gut-barrier enhancement, (viii) nitric oxide reduction, (ix) hormones production, (x) acetate production or (xi) any combinations thereof.

In accordance with some embodiments, the microbial consortium comprising two or more microorganisms, at least one of the two or more microorganisms is capable of having one or more of (i) putrescine production, (ii) vitamin B6 production, (iii) GABA production, (iv) secondary bile acid production, (v) pollutants detoxification, (vi) acetate production, (vii) gut-barrier enhancement, (viii) nitric oxide reduction, (ix) progesterone production, (x) acetate production or (xi) any combinations thereof.

In accordance with some embodiments, the microbial consortium comprising two or more microorganisms, at least one of the two or more microorganisms is capable of having one or more of (i) acetate production, (ii) progesterone production, or (iii) any combinations thereof.

In accordance with some embodiments, the microbial consortium comprising two or more microorganisms, at least one of the two or more microorganisms is capable of producing progesterone.

In accordance with some embodiments, the microbial consortium comprising two or more microorganisms, at least one of the two or more microorganisms is capable of having one or more of (i) GABA production, (ii) decreasing activity of at least one serine protease, (iii) pollutants detoxification (iv) acetate production (v) butyrate production, (vi) propionate production (vii) decreasing methanogenesis, (viii) decreasing sulphate reduction, (ix) isoflavonoids metabolism, or (x) any combinations thereof.

In accordance with some embodiments, the microbial consortium comprising two or more microorganisms, at least one of the two or more microorganisms is capable of producing GABA In accordance with some embodiments, the microbial consortium comprising two or more microorganisms, at least one of the two or more microorganisms is capable of metabolizing isoflavonoids.

In accordance with some embodiments, the microbial consortium comprising two or more microorganisms, said two or more microorganisms are capable of modulating one or more of (i) GABA, (ii) putrescine, (iii) glutamate, (iv) valerate, (v) isovalerate (vi) vitamin B6, (vii) acetate, (viii) butyrate, (ix) propionate, (x) ammonia, (xi) lactate, (xii) methane, (xiii) H₂S, (xiv) decreasing activity of at least one serine protease, (xv) vitamin B12 (xvi) arginine, (xvii) caproate, (xviii) iron sequestering, (xix) gut-barrier, (xx) linoleic acid, (xxi) serotonine production or (xxii) any combinations thereof.

In accordance with some embodiments, the microbial consortium comprising two or more microorganisms, said two or more microorganisms are capable of modulating one or more of (i) GABA, (ii) putrescine, (iii) glutamate, (iv) valerate, (v) isovalerate (vii) vitamin B6, (viii) acetate, (ix) butyrate, (x) propionate, (xi) ammonia, (xii) lactate, (xiii) methane, (xiv) H₂S, (xv) decreasing activity of at least one serine protease, (xvi) vitamin B12 (xvii) arginine, (xviii) caproate, (xix) linoleic acid or (xx) any combinations thereof.

In accordance with some embodiments, the microbial consortium comprising two or more microorganisms, said two or more microorganisms are capable of having one or more of (i) increase GABA levels, (ii) increase putrescine levels, (iii) increase glutamate level, (iv) increase valerate level, (v) increase isovalerate level (vi) increase vitamin B6 level, (vii) increase acetate level, (viii) increase butyrate level, (ix) increase propionate level, (x) increase ammonia level, (xi) increase lactate level, (xii) decrease methane level, (xiii) decrease H₂S levels, (xiv) decreasing activity of at least one serine protease, (xv) increase vitamin B12 level (xvi) increase arginine level, (xvii) increase caproate level, (xviii) increasing linoleic acid levels, (xix) increasing serotonine production or (xx) any combinations thereof.

In accordance with some embodiments, the microbial consortium comprising two or more microorganisms, said two or more microorganisms are capable of having one or more of (i) increase GABA levels, (ii) increase putrescine levels, (iii) increase glutamate level, (iv) increase valerate level, (v) increase isovalerate level (vi) increase vitamin B6 level, (vii) increase acetate level, (viii) increase butyrate level, (ix) increase propionate level, (x) increase ammonia level, (xi) increase lactate level, (xii) decrease methane level, (xiii) reduce H₂S levels, (xiv) decreasing activity of at least one serine protease, (xv) increase vitamin B12 level (xvi) increase arginine level, (xvii) increase caproate level, (xviii) increasing linoleic acid levels, (xix) increasing serotonin production or (xx) any combinations thereof.

In accordance with some embodiments, the microbial consortium comprising two or more microorganisms, the two or more microorganisms are capable of having one or more of (i) increase GABA levels, (ii) increase valerate level, (iii) increase isovalerate level, (iv) increase acetate level, (v) increase butyrate level, (vi) increase propionate level, (vii) increase serotonin production or (viii) any combinations thereof.

In accordance with some embodiments, the microbial consortium comprising two or more microorganisms, said two or more microorganisms are capable of modulating one or more of (i) putrescine, (ii) vitamin B6, (iii) iron sequestering, (iv) GABA (v) valerate, (vi) isovalerate (vii) lactate, (viii) caproate, (ix) bile acid, (x) pollutants, (xi) acetate, (xii) butyrate, (xiii) propionate, (xiv) ammonia, (xv) methane, (xvi) H₂S, (xvii) Treg, (xviii) nitric oxide, (xix) hormones, (xx) gut-barrier, (xxi) linoleic acid, (xxii) vitamin B12 or (xxiii) any combinations thereof.

In accordance with some embodiments, the microbial consortium comprising two or more microorganisms, said two or more microorganisms are capable of modulating one or more of (i) putrescine, (ii) vitamin B6, (iii) hormone, (iv) GABA (v) valerate, (vi) isovalerate (vii) lactate, (viii) caproate, (ix) bile acid, (x) pollutants, (xi) acetate, (xii) butyrate, (xiii) propionate, (xiv) ammonia, (xv) methane, (xvi) H₂S, (xvii) Treg, (xviii) nitric oxide (xix) linoleic acid, (xx) vitamin B12 or (xxi) any combinations thereof. In accordance with some embodiments, the microbial consortium comprising two or more microorganisms, said two or more microorganisms are capable of having one or more of (i) putrescine production, (ii) vitamin B6 production, (iii) hormone production, (iv) GABA production (v) valerate production, (vi) isovalerate production (vii) lactate production, (viii) caproate production, (ix) secondary bile acid production, (x) pollutants detoxification, (xi) acetate production, (xii) butyrate production, (xiii) propionate production, (xiv) ammonia production, (xv) decrease in methane level, (xvi) decrease in H₂S levels, (xvii) Treg induction, (xviii) nitric oxide reduction, (xix) increasing linoleic acid levels, (xx) vitamin B12 production or (xxi) any combinations thereof.

In accordance with some embodiments, the microbial consortium comprising two or more microorganisms, said two or more microorganisms are capable of having one or more of (i) putrescine production, (ii) vitamin B6 production, (iii) progesterone production, (iv) GABA production (v) valerate production, (vi) isovalerate production (vii) lactate production, (viii) caproate production, (ix) secondary bile acid production, (x) pollutants detoxification, (xi) acetate production, (xii) butyrate production, (xiii) propionate production, (xiv) ammonia production, (xv) decrease in methane level, (xvi) decrease in H₂S levels, (xvii) Treg induction, (xviii) nitric oxide reduction, (xix) increasing linoleic acid levels, (xx) vitamin B12 production or (xxi) any combinations thereof.

In accordance with some embodiments, the microbial consortium comprising two or more microorganisms, aid two or more microorganisms are capable of having one or more of (i) increase acetate level, (ii) increase butyrate level, (iii) increase propionate level, (iv) valerate production, (v) isovalerate production, (vi) progesterone production or (vii) any combinations thereof.

In accordance with some embodiments, the microbial consortium comprising two or more microorganisms, at least one of the two or more microorganisms is capable of modulating one or more of (i) GABA, (ii) putrescine, (iii) glutamate, (iv) valerate, (v) isovalerate (vi) caproate, (vii) vitamin B6, (viii) acetate, (ix) butyrate, (x) propionate, (xi) ammonia, (xii) lactate, (xiii) methane, (xiv) H₂S, (xv) serine protease activity, (xvi) vitamin B12 (xvii) arginine, (xviii) iron sequestering, (xix) isoflavonoids, (xx) pollutants, (xxi) gut-barrier, (xxii) linoleic acid or (xxiii) any combinations thereof.

In accordance with some embodiments, the microbial consortium comprising two or more microorganisms, at least one of the two or more microorganisms is capable of modulating one or more of (i) GABA, (ii) putrescine, (iii) glutamate, (iv) valerate, (v) isovalerate, (vi) caprotate, (vii) vitamin B6, (viii) acetate, (ix) butyrate, (x) propionate, (xi) ammonia, (xii) lactate, (xiii) methane, (xiv) H₂S, (xv) serine protease activity, (xvi) vitamin B12 (xvii) arginine, (xviii) pollutants, (xix) isoflavonoids, (xx) linoleic acid, or (xxi) any combinations thereof.

In accordance with some embodiments, the microbial consortium comprising two or more microorganisms, at least one of the two or more microorganisms is capable of having one or more of (i) GABA production, (ii) putrescine production, (iii) glutamate production, (iv) valerate production, (v) isovalerate production (vi) vitamin B6 production, (vii) acetate production, (viii) butyrate production, (ix) propionate production, (x) ammonia production, (xi) lactate production, (xii) decrease in methane level, (xiii) decrease in H₂S levels, (xiv) decreasing activity of at least one serine protease, (xv) vitamin B12 production (xvi) arginine production, (xvii) pollutants detoxification, (xviii) isoflavonoids metabolism, (xix) caproate production or (xx) increasing linoleic acid levels or (xxi) any combinations thereof.

In accordance with some embodiments, the microbial consortium comprising two or more microorganisms, at least one of the two or more microorganisms is capable of having one or more of (i) valerate production, (ii) isovalerate production (iii) acetate production, (iv) butyrate production, (v) propionate production, (vi) GABA production or (vii) any combinations thereof.

In accordance with some embodiments, the microbial consortium comprising two or more microorganisms, at least one of the two or more microorganisms is capable of modulating one or more of (i) GABA, (ii) putrescine, (iii) bile acid, (iv) valerate, (v) isovalerate (vi) vitamin B6, (vii) acetate, (viii) butyrate, (ix) propionate, (x) ammonia, (xi) lactate, (xii) methane, (xiii) H₂S, (xiv) serine protease activity, (xv) vitamin B12 (xvi) nitric oxide, (xvii) caproate, (xviii) isoflavonoids, (xix) pollutants detoxification, (xx) hormones, (xxi) iron sequestering, (xxii) gut-barrier, (xxiii) linoleic acid, (xiv) serotonin production or (xv) any combinations thereof.

In accordance with some embodiments, the microbial consortium comprising two or more microorganisms, at least one of the two or more microorganisms is capable of modulating one or more of (i) GABA, (ii) putrescine, (iii) bile acid, (iv) valerate, (v) isovalerate (vi) vitamin B6, (vii) acetate, (viii) butyrate, (ix) propionate, (x) ammonia, (xi) lactate, (xii) methane, (xiii) H₂S, (xiv) serine protease activity, (xv) vitamin B12 (xvi) nitric oxide, (xvii) caproate, (xviii) isoflavonoids, (xix) pollutants detoxification, (xx) hormones (xxi) linoleic acid. (xxii) serotonin production or (xxiii) any combinations thereof.

In accordance with some embodiments, the microbial consortium comprising two or more microorganisms, at least one of the two or more microorganisms is capable of having one or more of (i) GABA production, (ii) putrescine production, (iii) secondary bile acid production, (iv) valerate production, (v) isovalerate production (vi) vitamin B6 production, (vii) acetate production, (viii) butyrate production, (ix) propionate production, (x) ammonia production, (xi) lactate production, (xii) decrease in methane level, (xviii) decrease in H₂S levels, (xiv) decreasing activity of at least one serine protease, (xv) vitamin B12 production (xvi) nitric oxide reduction, (xvii) caproate production, (xviii) isoflavonoids metabolism, (xix) pollutants detoxification, (xx) hormones production, (xxi) increasing linoleic acid levels or (xxii) any combinations thereof.

In accordance with some embodiments, the microbial consortium comprising two or more microorganisms, at least one of the two or more microorganisms is capable of having one or more of (i) GABA production, (ii) putrescine production, (iii) secondary bile acid production, (iv) valerate production, (v) isovalerate production (vi) vitamin B6 production, (vii) acetate production, (viii) butyrate production, (ix) propionate production, (x) ammonia production, (xi) lactate production, (xii) decrease in methane level, (xviii) decrease in H₂S levels, (xiv) decreasing activity of at least one serine protease, (xv) vitamin B12 production (xvi) nitric oxide reduction, (xvii) caproate production, (xviii) isoflavonoids metabolism, (xix) pollutants detoxification, (xx) progesterone production, (xxi) increasing linoleic acid levels, (xxii) serotonin production or (xxiii) any combinations thereof.

In accordance with some embodiments, the microbial consortium comprising two or more microorganisms, at least one of the two or more microorganisms is capable of having one or more of (i) GABA production, (ii) valerate production, (iii) isovalerate production (iv) acetate production, (v) butyrate production, (vi) propionate production, (vii) serotonin production, (viii) progesterone production or (ix) any combinations thereof.

In accordance with some embodiments, the microbial consortium comprising two or more microorganisms, at least one of the two or more microorganisms is capable of modulating one or more of (i) GABA, (ii) putrescine, (iii) glutamate, (iv) valerate, (v) isovalerate (vi) caproate, (vii) vitamin B6, (viii) acetate, (ix) butyrate, (x) propionate, (xi) ammonia, (xii) lactate, (xiii) methane, (xiv) H₂S, (xv) serine protease activity, (xvi) vitamin B12 (xvii) arginine, (xviii) iron sequestering, (xix) isoflavonoids, (xx) pollutants detoxification, (xxi) gut-barrier, (xxii) linoleic acid, (xxiii) bile acid, (xxiv) nitric oxide, (xxv) isoflavonoids, (xxvi) hormones or (xxvii) any combinations thereof.

In accordance with some embodiments, the microbial consortium comprising two or more microorganisms, at least one of the two or more microorganisms is capable of having one or more of (i) valerate production, (ii) isovalerate production (iii) acetate production, (iv) butyrate production, (v) propionate production, (vi) GABA production or (vii) any combinations thereof.

In some examples, the microbial consortium comprising two or more microorganisms, at least one of the two or more microorganisms is capable of having one or more of (i) production of vitamin B6 (ii) acetate production, (iii) nitric oxide reduction (degradation), (iv) production of progesterone (v) pollutants detoxification or (vi) any combinations thereof.

As described herein, the microbial consortium comprises at least two microorganisms, each as defined herein or any combinations of each one of the microorganism described herein.

When referring to a microbial consortium comprising at least two microorganisms it should be understood as referring to two different microorganisms (i.e. different strains). The two different microorganisms may be within the same microbial genus or within the microorganism species.

For examples, the two or more microorganisms can belong both to one genus but to different species within or alternatively to the same genus, the same species but different strains. Alternatively, the two or more microorganisms can belong each to a different genus.

In some embodiments, the microbial consortium comprises one or more microorganism from the Megasphaera genus, Lactobacillus genus, Dialister genus, Bifidobacterium genus, Eubacterium genus, Megamonas genus, Oscillibacter genus, Hafnia genus, unclassified Ruminococcaceae genus, Acetoanaerobium genus, Clostridium genus, Coprococcus genus, Roseburia genus, Mitsuokella genus, Selenomonas genus, Phascolarctobacterium genus, Blautia genus, unclassified Enterobacteriaceae genus, Bacillus genus, Cellulosilyticum genus, Pelosinus genus, Fructilactobacillus genus, Anaeroglobus genus or combinations thereof.

In some embodiments, the microbial consortium comprises one or more microorganism from the Megasphaera genus, Lactobacillus genus, Dialister genus, Bifidobacterium genus, Eubacterium genus, Megamonas genus, Oscillibacter genus, Hafnia genus, unclassified Ruminococcaceae genus, Acetoanaerobium genus, Clostridium genus, Coprococcus genus, Roseburia genus, Mitsuokella genus, Selenomonas genus, Phascolarctobacterium genus, Blautia genus, or combinations thereof.

In some embodiments, the microbial consortium comprises two or more microorganisms belonging to one or more of the Megasphaera genus, Lactobacillus genus, Dialister genus, Bifidobacterium genus, Eubacterium genus, Megamonas genus, Oscillibacter genus, Hafnia genus, or combinations thereof.

In some embodiments, the microbial consortium comprises at least one microorganism from the Megasphaera genus.

In some other embodiments, the microbial consortium comprises at least one microorganism from the Lactobacillus genus.

In some embodiments, the microbial consortium comprises at least one microorganism from the Dialister genus

In some embodiments, the microbial consortium comprises at least one microorganism from the Bifidobacterium genus

In some embodiments, the microbial consortium comprises at least one microorganism from the Eubacterium genus.

In some embodiments, the microbial consortium comprises at least one microorganism from the Oscillibacter genus.

In some embodiments, the microbial consortium comprises at least one microorganism from the Hafnia genus.

In some embodiments, the microbial consortium comprises two or more microorganisms belonging to one or more of the Megasphaera genus, Dialister genus, Lactobacillus genus, Eubacterium genus, Bifidobacterium genus, or combinations thereof.

In some embodiments, the microbial consortium comprises two or more microorganisms belonging to one or more of the Megasphaera genus, Hafnia genus, Lacobacillus genus, Oscillibacter genus, Megamonas genus, or combinations thereof.

In some embodiments, the microbial consortium comprises two or more microorganisms belonging to one or more of the Megasphaera genus, Dialister genus, Eubacterium genus, Megamonas genus or combinations thereof.

In some embodiments, the microbial consortium comprises two or more microorganisms belonging to one or more of the Megasphaera genus, Lactobacillus genus, Eubacterium genus, Hafnia genus, Bifidobacterium genus, or combinations thereof.

In some embodiments, the microbial consortium comprises two or more microorganisms belonging to one or more of the Megasphaera genus, Dialister genus, Lactobacillus genus, Eubacterium genus, Hafnia genus, Bifidobacterium genus, Megamonas genus or combinations thereof.

In some embodiments, the microbial consortium comprises at least one microorganism from the Megasphaera genus. Megasphaera genus may be denoted by Taxonomy ID: 906.

In some embodiments, the microbial consortium comprises at least one microorganism from the Dialister genus. Dialister genus may be denoted by Taxonomy ID: 39948.

In some embodiments, the microbial consortium comprises at least one microorganism from the Bifidobacterium genus. Bifidobacterium genus may be denoted by Taxonomy ID: 1678. Bifidobacterium is a genus of Gram-positive bacteria.

In some embodiments, the microbial consortium comprises at least one microorganism from the Oscillibacter genus. Oscillibacter genus may be denoted by Taxonomy ID: 459786. Oscillibacter is a genus of Gram-negative bacteria.

In some embodiments, the microbial consortium comprises at least one microorganism from the Hafnia genus. Hafnia genus may be denoted by Taxonomy ID: 568. Hafnia is a genus of Gram-negative bacteria.

In some embodiments, the microbial consortium comprises at least one microorganism from the Megamonas genus. Megamonas genus may be denoted by Taxonomy ID: 158846.

In some embodiments, the microbial consortium comprises at least one microorganism from the Eubacterium genus. Eubacterium genus may be denoted by Taxonomy ID (or taxid): 1730. Eubacterium is a genus of Gram-positive bacteria and are characterized by a rigid cell wall.

In some embodiments, the microbial consortium comprises at least one microorganism from the Lactobacillus genus. Lactobacillus genus may be denoted by Taxonomy ID (or taxid): 1578.

In some embodiments, the microbial consortium comprises at least one microorganism from the Coprococcus genus. Coprococcus genus may be denoted by Taxonomy ID (or taxid): 33042.

In some embodiments, the microbial consortium comprises at least one microorganism from the Anaeroglobus genus. Anaeroglobus genus may be denoted by Taxonomy ID (or taxid): 156454.

In some embodiments, the microbial consortium comprises at least one microorganism from the Roseburia genus. Roseburia genus may be denoted by Taxonomy ID (or taxid): 841.

In some embodiments, the microbial consortium comprises at least one microorganism from the Selenomonas genus. Selenomonas genus may be denoted by Taxonomy ID (or taxid): 970.

In some embodiments, the microbial consortium comprises at least one microorganism from the Mitsuokella genus. Mitsuokella genus may be denoted by Taxonomy ID (or taxid): 52225.

In some embodiments, the microbial consortium comprises at least one microorganism from the Phascolarctobacterium genus. Phascolarctobacterium genus may be denoted by Taxonomy ID (or taxid): 33024.

In some embodiments, the microbial consortium comprises at least one microorganism from the Acetobacterium genus. Acetobacterium genus may be denoted by Taxonomy ID (or taxid): 33951.

In some embodiments, the microbial consortium comprises at least one microorganism from the Blautia genus. Blautia genus may be denoted by Taxonomy ID (or taxid): 572511.

In some embodiments, the microbial consortium comprises at least one microorganism from the Clostridium genus. Clostridium genus may be denoted by Taxonomy ID (or taxid): 1485.

In some embodiments, the microbial consortium comprises at least one microorganism from the unclassified Ruminococcaceae genus, from the family of Ruminococcaceae. Unclassified Ruminococcaceae genus may be denoted by Taxonomy ID (or taxid): 2305133.

In some embodiments, the microbial consortium comprises at least one microorganism from the Bacillus genus. Bacillus genus may be denoted by Taxonomy ID (or taxid): 1386.

In some embodiments, the microbial consortium comprises at least one microorganism from the Cellulosilyticum genus. Cellulosilyticum genus may be denoted by Taxonomy ID (or taxid): 698776.

In some embodiments, the microbial consortium comprises at least one microorganism from the Pelosinus genus. Pelosinus genus may be denoted by Taxonomy ID (or taxid): 365348.

In some embodiments, the microbial consortium comprises at least one microorganism from the Fructilactobacillus genus. Fructilactobacillus genus may be denoted by Taxonomy ID (or taxid): 2767881.

In some embodiments, the microbial consortium comprises at least one microorganism from the unclassified Enterobacteriaceae genus from the family of the Enterobacteriaceae. Unclassified Enterobacteriaceae Genus, from the family Enterobacteriaceae may be denoted by Taxonomy ID (or taxid): 36866.

In some embodiments, the microbial consortium comprises at least one, at least two or more microorganisms selected from the Megasphaera elsdenii species, Lactobacillus ruminis species, Dialister invisus species, Bifidobacterium dentium species, Eubacterium siraeum species, Megamonas funiformis species, Oscillibacter valericigenes species, Hafnia alvei species, Eubacterium limosum species, Megasphaera sp. species, Megasphaera hexanoica species, Eubacterium hallii species, Acetoanaerobium sticklandii species, Ruminococcaceae bacterium species, Clostridium kluyveri species, Coprococcus comes species, Coprococcus catus species, Roseburia inulinivorans species, Lactobacillus rhamnosus species, Lactobacillus reuteri species, Lactobacillus plantarum species, Lactobacillus salivarius species, Dialister succinatiphilus species, Bifidobacterium adolescentis species, Mitsuokella multacida species, Selenomonas sputigena species, Phascolarctobacterium succinatutens species, Phascolarctobacterium faecium species, Oscillibacter sp. species, Eubacterium callanderi species, Acetobacterium woodii species, Blautia producta species, Blautia hydrogenotrophica species, Megasphaera massiliensis species, Megasphaera stantonii species, Anaeroglobus geminatus species, Megasphaera micronuciformis species, Lactobacillus murinus species, Bacillus circulans species, Dialister massiliensis species, Bifidobacterium pseudocatenulatum species, Cellulosilyticum lentocellum species, Selenomonas ruminantium subsp. lactilytica species, Pelosinus fermentans species, Enterobacteriaceae bacterium 9_2_54FAA species, Clostridium beijerinckii species, Fructilactobacillus sanfranciscensis species, subspecies or any combinations thereof.

In some embodiments, the microbial consortium comprises at least one, at least two or more microorganisms selected from Megasphaera elsdenii species, Lactobacillus ruminis species, Dialister invisus species, Bifidobacterium dentium species, Eubacterium siraeum species, Megamonas funiformis species, Oscillibacter valericigenes species, Hafnia alvei species, Eubacterium limosum species, Megasphaera sp. species, Megasphaera hexanoica species, Eubacterium hallii species, Acetoanaerobium sticklandii species, Ruminococcaceae bacterium species, Clostridium kluyveri species, Coprococcus comes species, Coprococcus catus species, Roseburia inulinivorans species, Lactobacillus rhamnosus species, Lactobacillus reuteri species, Lactobacillus plantarum species, Lactobacillus salivarius species, Dialister succinatiphilus species, Bifidobacterium adolescentis species, Mitsuokella multacida species, Selenomonas sputigena species, Phascolarctobacterium succinatutens species, Phascolarctobacterium faecium species, Oscillibacter sp. species, Eubacterium callanderi species, Acetobacterium woodii species, Blautia producta species, Blautia hydrogenotrophica species, subspecies or any combinations thereof.

In some embodiments, the microbial consortium comprises at least one, at least two or more microorganisms selected from Megasphaera stantonii species, Anaeroglobus geminatus species, Megasphaera micronuciformis species, Lactobacillus murinus species, Bacillus circulans species, Dialister massiliensis species, Bifidobacterium pseudocatenulatum species, Cellulosilyticum lentocellum species, Selenomonas ruminantium subsp. lactilytica species, Pelosinus fermentans species, Enterobacteriaceae bacterium 9_2_54FAA species, Clostridium beijerinckii species, Fructilactobacillus sanfranciscensis species, Megasphaera massiliensis species subspecies or any combinations thereof.

In some embodiments, the microbial consortium comprises at least one microorganism from the Megasphaera elsdenii species.

In some embodiments, the microbial consortium comprises at least two microorganisms species belonging to species selected from Megasphaera elsdenii species, Lactobacillus ruminis species, Dialister invisus species, Bifidobacterium dentium species, Eubacterium siraeum species, Megamonas funiformis species, Oscillibacter valericigenes species, Hafnia alvei species, Eubacterium limosum species, subspecies or any combinations thereof.

In some embodiments, the microbial consortium comprises at least two microorganisms species belonging to species selected from Megasphaera elsdenii species, Lactobacillus ruminis species, Dialister invisus species, Bifidobacterium dentium species, Eubacterium siraeum species, subspecies or any combinations thereof.

In some embodiments, the microbial consortium comprises at least two microorganisms species belonging to species selected from Megasphaera elsdenii species, Lactobacillus ruminis species, Megamonas funiformis species, Oscillibacter valericigenes species, Hafnia alvei species, subspecies or any combinations thereof.

In some embodiments, the microbial consortium comprises at least two microorganisms species belonging to species selected from Megasphaera elsdenii species, Dialister invisus species, Eubacterium limosum species, Megamonas funiformis species, subspecies or any combinations thereof.

In some embodiments, the microbial consortium comprises two or more microorganisms belonging to one or more of the Megasphaera elsdenii species, Lactobacillus ruminis species, Eubacterium limosum species, Hafnia alvei species, Bifidobacterium dentium species, subspecies or any combinations thereof.

In some embodiments, the microbial consortium comprises two or more microorganisms belonging to one or more of the Megasphaera elsdenii species, Dialister invisus species, Eubacterium limosum species, Megamonas funiformis species, Lactobacillus ruminis species, Hafnia alvei species, Bifidobacterium dentium species subspecies or any combinations thereof.

In some embodiments, the microbial consortium comprises at least two microorganisms species belonging to species selected from Megasphaera elsdenii species, Lactobacillus ruminis species, subspecies or any combinations thereof.

In some embodiments, the microbial consortium comprises at least two microorganisms species belonging to species selected from Megasphaera elsdenii species, Lactobacillus ruminis species, Dialister succinatiphilus species, Bifidobacterium dentium species, Eubacterium siraeum species, subspecies or any combinations thereof.

In some embodiments, the microbial consortium comprises at least two microorganisms species belonging to species selected from Megasphaera elsdenii species, Dialister succinatiphilus species, Eubacterium limosum species, Megamonas funiformis species, subspecies or any combinations thereof.

In some embodiments, the microbial consortium comprises at least one microorganism from the Megasphaera elsdenii species. Megasphaera elsdenii species may be denoted by Taxonomy ID: 907.

In some embodiments, the microbial consortium comprises at least one microorganism from the Lactobacillus ruminis species. Lactobacillus ruminis species may be denoted by Taxonomy ID: 1623.

In some embodiments, the microbial consortium comprises at least one microorganism from the Dialister invisus species. Dialister invisus species may be denoted by Taxonomy ID: 218538.

In some embodiments, the microbial consortium comprises at least one microorganism from the Bifidobacterium dentium species. Bifidobacterium dentium species may be denoted by Taxonomy ID: 1689.

In some embodiments, the microbial consortium comprises at least one microorganism from the Eubacterium siraeum species. Eubacterium siraeum species may be denoted by Taxonomy ID: 39492.

In some embodiments, the microbial consortium comprises at least one microorganism from the Megamonas funiformis species. Megamonas funiformis species may be denoted by Taxonomy ID: 437897.

In some embodiments, the microbial consortium comprises at least one microorganism from the Oscillibacter valericigenes species. Oscillibacter valericigenes species may be denoted by Taxonomy ID: 351091.

In some embodiments, the microbial consortium comprises at least one microorganism from the Hafnia alvei species. Hafnia alvei species may be denoted by Taxonomy ID: 569.

In some embodiments, the microbial consortium comprises at least one microorganism from the Eubacterium limosnitrateum species. Eubacterium limosum species may be denoted by Taxonomy ID: 1736.

In some embodiments, the microbial consortium comprises at least one microorganism from the Megasphaera sp. species. Megasphaera sp. species may be denoted by Taxonomy ID: 2023260.

In some embodiments, the microbial consortium comprises at least one microorganism from the Megasphaera hexanoica species. Megasphaera hexanoica species may be denoted by Taxonomy ID: 1675036.

In some embodiments, the microbial consortium comprises at least one microorganism from the Eubacterium hallii species. Eubacterium hallii species may be denoted by Taxonomy ID: 39488.

In some embodiments, the microbial consortium comprises at least one microorganism from the Acetoanaerobium sticklandii species. Acetoanaerobium sticklandii species may be denoted by Taxonomy ID: 1511.

In some embodiments, the microbial consortium comprises at least one microorganism from the Ruminococcaceae bacterium species. Ruminococcaceae bacterium species may be denoted by Taxonomy ID: 1898205.

In some embodiments, the microbial consortium comprises at least one microorganism from the Clostridium kluyveri species. Clostridium kluyveri species may be denoted by Taxonomy ID: 1534.

In some embodiments, the microbial consortium comprises at least one microorganism from the Coprococcus comes species. Coprococcus comes species may be denoted by Taxonomy ID: 410072.

In some embodiments, the microbial consortium comprises at least one microorganism from the Coprococcus catus species. Coprococcus catus species may be denoted by Taxonomy ID: 116085.

In some embodiments, the microbial consortium comprises at least one microorganism from the Roseburia inulinivorans species. Roseburia inulinivorans species may be denoted by Taxonomy ID: 360807.

In some embodiments, the microbial consortium comprises at least one microorganism from the Lactobacillus rhamnosus species. Lactobacillus rhamnosus species may be denoted by Taxonomy ID: 47715.

In some embodiments, the microbial consortium comprises at least one microorganism from the Lactobacillus reuteri species. Lactobacillus reuteri species may be denoted by Taxonomy ID: 1598.

In some embodiments, the microbial consortium comprises at least one microorganism from the Lactobacillus plantarum species. Lactobacillus plantarum species may be denoted by Taxonomy ID: 1590.

In some embodiments, the microbial consortium comprises at least one microorganism from the Lactobacillus salivarius species. Lactobacillus salivarius species may be denoted by Taxonomy ID: 1624.

In some embodiments, the microbial consortium comprises at least one microorganism from the Dialister succinatiphilus species. Dialister succinatiphilus species may be denoted by Taxonomy ID: 487173.

In some embodiments, the microbial consortium comprises at least one microorganism from the Bifidobacterium adolescentis species. Bifidobacterium adolescentis species may be denoted by Taxonomy ID: 1680.

In some embodiments, the microbial consortium comprises at least one microorganism from the Mitsuokella multacida species. Mitsuokella multacida species may be denoted by Taxonomy ID: 52226.

In some embodiments, the microbial consortium comprises at least one microorganism from the Selenomonas sputigena species. Selenomonas sputigena species may be denoted by Taxonomy ID: 69823.

In some embodiments, the microbial consortium comprises at least one microorganism from the Phascolarctobacterium succinatutens species. Phascolarctobacterium succinatutens species may be denoted by Taxonomy ID: 626940.

In some embodiments, the microbial consortium comprises at least one microorganism from the Phascolarctobacterium faecium species. Phascolarctobacterium faecium species may be denoted by Taxonomy ID: 33025.

In some embodiments, the microbial consortium comprises at least one microorganism from the Oscillibacter sp., species. Oscillibacter sp. species may be denoted by Taxonomy ID: 1945593.

In some embodiments, the microbial consortium comprises at least one microorganism from the Eubacterium callanderi species. Eubacterium callanderi species may be denoted by Taxonomy ID: 53442.

In some embodiments, the microbial consortium comprises at least one microorganism from the Acetobacterium woodii species. Acetobacterium woodii species may be denoted by Taxonomy ID: 33952.

In some embodiments, the microbial consortium comprises at least one microorganism from the Blautia producta species. Blautia producta species may be denoted by Taxonomy ID: 33035.

In some embodiments, the microbial consortium comprises at least one microorganism from the Blautia hydrogenotrophica species. Blautia hydrogenotrophica species may be denoted by Taxonomy ID: 53443.

In some embodiments, the microbial consortium comprises at least one microorganism from the Megasphaera stantonii species. Megasphaera stantonii species may be denoted by Taxonomy ID: 2144175.

In some embodiments, the microbial consortium comprises at least one microorganism from the Anaeroglobus geminatus species. Anaeroglobus geminatus species may be denoted by Taxonomy ID: 156456.

In some embodiments, the microbial consortium comprises at least one microorganism from the Megasphaera micronuciformis species. Megasphaera micronuciformis species may be denoted by Taxonomy ID: 187326.

In some embodiments, the microbial consortium comprises at least one microorganism from the Lactobacillus murinus species. Lactobacillus murinus species may be denoted by Taxonomy ID: 1622.

In some embodiments, the microbial consortium comprises at least one microorganism from the Bacillus circulans species. Bacillus circulans species may be denoted by Taxonomy ID: 1397.

In some embodiments, the microbial consortium comprises at least one microorganism from the Dialister massiliensis species. Dialister massiliensis species may be denoted by Taxonomy ID: 2161821.

In some embodiments, the microbial consortium comprises at least one microorganism from the Bifidobacterium pseudocatenulatum species. Bifidobacterium pseudocatenulatum species may be denoted by Taxonomy ID: 28026.

In some embodiments, the microbial consortium comprises at least one microorganism from the Cellulosilyticum lentocellum species. Cellulosilyticum lentocellum species may be denoted by Taxonomy ID: 29360.

In some embodiments, the microbial consortium comprises at least one microorganism from the Selenomonas ruminantium subsp. lactilytica species. Selenomonas ruminantium subsp. lactilytica species may be denoted by Taxonomy ID: 114197.

In some embodiments, the microbial consortium comprises at least one microorganism from the Pelosinus fermentans species. Pelosinus fermentans species may be denoted by Taxonomy ID: 365349.

In some embodiments, the microbial consortium comprises at least one microorganism from the Enterobacteriaceae bacterium 9_2_54FAA species. Enterobacteriaceae bacterium 9_2_54FAA species may be denoted by Taxonomy ID: 469613.

In some embodiments, the microbial consortium comprises at least one microorganism from the Clostridium beijerinckii species. Clostridium beijerinckii species may be denoted by Taxonomy ID: 1520.

In some embodiments, the microbial consortium comprises at least one microorganism from the Fructilactobacillus sanfranciscensis species. Fructilactobacillus sanfranciscensis species may be denoted by Taxonomy ID: 1625.

In some embodiments, the microbial consortium comprises at least one microorganism from the Megasphaera massiliensis species. Megasphaera massiliensis species may be denoted by Taxonomy ID: 1232428.

In the context of the present disclosure, identification of microorganism from a biological sample of a human subject may be done using any conventional method in the microbiology field. For example, and without being limited to identification of bacteria from a biological sample of a human subject may be done using 16S rRNA (ribosomal RNA) sequencing. Identification of isolated microorganism may be done by conducting similarity analysis between the 16S rRNA gene of the isolated microorganism to a different microorganism's 16S rRNA gene sequences available in a database. This analysis may be done in order to explore the similarity between a given sequence and all of the available sequences in a database and obtaining the best matched sequences by calculation of a score for the examined similarity. Identity analysis may be conducted by any appropriate program, for example Basic Local Alignment Search Tool (BLAST®) using publicly available databases, for example National Center for Biotechnology Information (NCBI).

It should be noted that the GenBank or Refseq accession Nos. provide below either provide the 16S rRNA sequence of the microorganism or the sequence of the entire genome. It should be further noted that a skilled person would know to evaluate the 16S rRNA sequence from the entire genome sequence.

In some embodiments, at least one of the two or more microorganisms comprises 16S rRNA sequences having at least 85%, at least 90%, at least 95%, at least 97%, at least 98%, at least 99% identity with at least one, at least two, at least three, at least four or more nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 or SEQ ID NO:40.

In some embodiments, at least one of the two or more microorganisms comprises 16S rRNA sequences having at least 85%, at least 90%, at least 95%, at least 97%, at least 98%, at least 99% identity with at least one, at least two, at least three, at least four or more nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

It should be noted that in the context of the invention, when referring to % identity each one microorganism from the at least two of the two or more microorganisms can have a different sequence identity from a corresponding sequence denoted above.

In some embodiments, at least one of the two or more microorganisms comprises 16S rRNA sequences having, between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:3, SEQ ID NO:4 and SEQ ID NO:5.

In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:3, SEQ ID NO:4 and SEQ ID NO:5.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:3, SEQ ID NO:4, SEQ ID NO:5 and SEQ ID NO:8.

In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:3, SEQ ID NO:4, SEQ ID NO:5 and SEQ ID NO:8.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:6, SEQ ID NO:7 and SEQ ID NO:8.

In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:6, SEQ ID NO:7 and SEQ ID NO:8.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:3, SEQ ID NO:6, and SEQ ID NO:9.

In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:3, SEQ ID NO:6, and SEQ ID NO:9.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:3, SEQ ID NO:6, SEQ ID NO:8 and SEQ ID NO:9.

In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:3, SEQ ID NO:6, SEQ ID NO:8 and SEQ ID NO:9.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:4, SEQ ID NO:8, and SEQ ID NO:9. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:4, SEQ ID NO:8, and SEQ ID NO:9.

In some embodiments, at least two of the two or more microorganisms comprises 16S rRNA sequences having at least 85%, at least 90%, at least 95%, at least 99% identity with at least two nucleic acid sequences denoted by SEQ ID NO:1 and SEQ ID NO:2. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1 and SEQ ID NO:2.

In some embodiments, at least one of the two or more microorganisms comprises 16S rRNA sequences having, between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:8 and at least one other (different) of the two or more microorganisms comprises 16S rRNA sequences having, between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7 and SEQ ID NO:9.

The term identity (% identity) as used herein refer to two or more nucleic acid sequences that are the same. In the context of the present disclosure the sequence identity encompasses transcription changes of DNA to RNA, e.g. T and U are considered identical. The identity may exist over a region of a sequence that is considered by those versed in the art as the variable region of the 16S rRNA. In some embodiments, the identity exists over the length the 16S rRNA or a portion thereof of the variable region.

The % identity between two or more nucleic acid sequences is determined for the two or more sequences when compared and aligned for maximum correspondence. In the context of the present disclosure, sequences (nucleic acid) as described herein having % identity are considered to have the same function/activity of the original sequence to which identity is calculated to.

The threshold sequence identity may be 85%, at times 86%, at times 87%, at times 88%, at times 89%, at times 90%, at times 91%, at times 92%, at times 93%, at times 94%, at times 95%, at times 96%, at times 97%, at times 98%, at times 99% with each one of the % identity denoted herein constitute a separate embodiment of the invention.

In some embodiments, the microbial consortium comprises at least Megasphaera elsdenii ATCC 17753. Megasphaera elsdenii ATCC 17753 may be denoted by Taxonomy ID: 1410663. The DNA sequence of Megasphaera elsdenii ATCC 17753 is provided by GenBank accession No. CP027569.1. The 16S rRNA sequence of Megasphaera elsdenii ATCC 17753 is denoted as SEQ ID NO:1.

In some embodiments, the microbial consortium comprises at least Lactobacillus ruminis ATCC 25644. Lactobacillus ruminis ATCC 25644 may be denoted by Taxonomy ID: 525362. The 16S rRNA sequence of Lactobacillus ruminis ATCC 25644 is provided by GenBank accession No. ACGS00000000.2 and denoted as SEQ ID NO:2.

In some embodiments, the microbial consortium comprises at least Dialister invisus DSM 15470. Dialister invisus DSM 15470 may be denoted by Taxonomy ID:592028. The 16S rRNA sequence of Dialister invisus DSM 15470 is provided by GenBank accession No. ACIM00000000.2 and denoted as SEQ ID NO:3.

In some embodiments, the microbial consortium comprises at least Bifidobacterium dentium ATCC 27679. Bifidobacterium dentium ATCC 27679 may be denoted by Taxonomy ID: 871562. The DNA sequence of Bifidobacterium dentium ATCC 27679 is provided by GenBank accession No. AEEQ00000000.1. The 16S rRNA sequence of Bifidobacterium dentium ATCC 27679 is denoted as SEQ ID NO:4.

In some embodiments, the microbial consortium comprises at least Eubacterium siraeum DSM 15702. Eubacterium siraeum DSM 15702 may be denoted by Taxonomy ID: 428128 The DNA sequence of Eubacterium siraeum DSM 15702 is provided by GenBank accession No. ABCA00000000.3. The 16S rRNA sequence of Eubacterium siraeum DSM 15702 is denoted as SEQ ID NO:5.

In some embodiments, the microbial consortium comprises at least Megamonas funiformis YIT 11815. Megamonas funiformis YIT 11815 may be denoted by Taxonomy ID: 742816. The DNA sequence of Megamonas funiformis YIT 11815 is provided by GenBank accession No. CP048627.1. The 16S rRNA sequence of Megamonas funiformis YIT 11815 is denoted as SEQ ID NO:6.

In some embodiments, the microbial consortium comprises at least Oscillibacter valericigenes DSM 18026. Oscillibacter valericigenes DSM 18026 may be denoted by Taxonomy ID: 693746. The 16S rRNA sequence of Oscillibacter valericigenes DSM 18026 is provided by GenBank accession No. AP012044.1 and denoted as SEQ ID NO:7.

In some embodiments, the microbial consortium comprises at least Hafnia alvei ATCC 51873. Hafnia alvei ATCC 51873 may be denoted by Taxonomy ID: 1002364. The DNA sequence of Hafnia alvei ATCC 51873 is provided by GenBank accession No. AGCI00000000.1. The 16S rRNA sequence of Hafnia alvei ATCC 51873 is denoted as SEQ ID NO:8.

In some embodiments, the microbial consortium comprises at least Eubacterium limosum ATCC 8486. Eubacterium limosum ATCC 8486 may be denoted by Taxonomy ID: 1736. The DNA sequence of Eubacterium limosum ATCC 8486 is provided by GenBank accession No. CP019962.1. The 16S rRNA sequence of Eubacterium limosum ATCC 8486 is denoted as SEQ ID NO:9.

In some embodiments, the microbial consortium comprises at least Megasphaera elsdenii ATCC 25940. Megasphaera elsdenii ATCC 25940 may be denoted by Taxonomy ID: 1064535. The DNA sequence of Megasphaera elsdenii ATCC 25940 is provided by GenBank accession No. HE576794.1. The 16S rRNA sequence of Megasphaera elsdenii ATCC 25940 is denoted as SEQ ID NO:10.

In some embodiments, the microbial consortium comprises at least Megasphaera sp. MJR8396C. Megasphaera sp. MJR8396C may be denoted by Taxonomy ID: 1603888. The DNA sequence of Megasphaera sp. MJR8396C is provided by GenBank accession No. LRVC00000000.1. The 16S rRNA sequence of Megasphaera sp. MJR8396C is denoted as SEQ ID NO:11.

In some embodiments, the microbial consortium comprises at least Megasphaera hexanoica JCM 31403. Megasphaera hexanoica JCM 31403 may be denoted by assembly number GCF_003315775.1. The DNA sequence of Megasphaera hexanoica JCM 31403 is provided by GenBank accession No. CP011940.1. The 16S rRNA sequence of Megasphaera hexanoica JCM 31403 is denoted as SEQ ID NO:12.

In some embodiments, the microbial consortium comprises at least Eubacterium hallii DSM 3353. Eubacterium hallii DSM 3353 may be denoted by Taxonomy ID: 411469. The DNA sequence of Eubacterium hallii DSM 3353 is provided by GenBank accession No. ACEP00000000.1. The 16S rRNA sequence of Eubacterium hallii DSM 3353 is denoted as SEQ ID NO:13.

In some embodiments, the microbial consortium comprises at least Acetoanaerobium sticklandii DSM 519. Acetoanaerobium sticklandii DSM 519 may be denoted by Taxonomy ID: 499177. The DNA sequence of Acetoanaerobium sticklandii DSM 519 is provided by GenBank accession No. NC_014614.1. The 16S rRNA sequence of Acetoanaerobium sticklandii DSM 519 is denoted as SEQ ID NO:14.

In some embodiments, the microbial consortium comprises at least Ruminococcaceae bacterium CPB6. Ruminococcaceae bacterium CPB6 may be denoted by assembly number GCF_002119605.1. The DNA sequence of Ruminococcaceae bacterium CPB6 is provided by GenBank accession No. CP020705.1. The 16S rRNA sequence of Ruminococcaceae bacterium CPB6 is denoted as SEQ ID NO:15.

In some embodiments, the microbial consortium comprises at least Clostridium kluyveri DSM 555. Clostridium kluyveri DSM 555 may be denoted by Taxonomy ID: 431943. The DNA sequence of Clostridium kluyveri DSM 555 is provided by GenBank accession No. CP000673.1. The 16S rRNA sequence of Clostridium kluyveri DSM 555 is denoted as SEQ ID NO:16.

In some embodiments, the microbial consortium comprises at least Coprococcus comes ATCC 27758. Coprococcus comes ATCC 27758 may be denoted by Taxonomy ID: 470146. The DNA sequence of Coprococcus comes ATCC 27758 is provided by GenBank accession No. ABVR00000000.1. The 16S rRNA sequence of Coprococcus comes ATCC 27758 is denoted as SEQ ID NO:17.

In some embodiments, the microbial consortium comprises at least Coprococcus catus GD/7. Coprococcus catus GD/7 may be denoted by Taxonomy ID: 717962. The DNA sequence of Coprococcus catus GD/7 is provided by GenBank accession No. FP929038.1. The 16S rRNA sequence of Coprococcus catus GD/7 is denoted as SEQ ID NO:18.

In some embodiments, the microbial consortium comprises at least Roseburia inulinivorans DSM 16841. Roseburia inulinivorans DSM 16841 may be denoted by Taxonomy ID: 622312. The DNA sequence of Roseburia inulinivorans DSM 16841 is provided by GenBank accession No. ACFY00000000.1. The 16S rRNA sequence of Roseburia inulinivorans DSM 16841 is denoted as SEQ ID NO:19.

In some embodiments, the microbial consortium comprises at least Lactobacillus ruminis ATCC 27782. Lactobacillus ruminis ATCC 27782 may be denoted by Taxonomy ID: 1069534. The DNA sequence of Lactobacillus ruminis ATCC 27782 is provided by GenBank accession No. CP003032.1. The 16S rRNA sequence of Lactobacillus ruminis ATCC 27782 is denoted as SEQ ID NO:20.

In some embodiments, the microbial consortium comprises at least Lactobacillus rhamnosus GG. Lactobacillus rhamnosus GG may be denoted by Taxonomy ID: 568703. The DNA sequence of Lactobacillus rhamnosus GG is provided by GenBank accession No. CP031290.1. The 16S rRNA sequence of Lactobacillus rhamnosus GG is denoted as SEQ ID NO:21.

In some embodiments, the microbial consortium comprises at least Lactobacillus reuteri ATCC 55730. Lactobacillus reuteri ATCC 55730 may be denoted by Taxonomy ID: 491077. The DNA sequence of Lactobacillus reuteri ATCC 55730 is provided by GenBank accession No. CP002844.1. The 16S rRNA sequence of Lactobacillus reuteri ATCC 55730 is denoted as SEQ ID NO:22.

In some embodiments, the microbial consortium comprises at least Lactobacillus plantarum subsp. plantarum LB1-2. Lactobacillus plantarum subsp. plantarum LB1-2 may be denoted by assembly number GCF_002906875.1. The DNA sequence of Lactobacillus plantarum subsp. plantarum LB1-2 is provided by GenBank accession No. CP025991.1. The 16S rRNA sequence of Lactobacillus plantarum subsp. plantarum LB1-2 is denoted as SEQ ID NO:23.

In some embodiments, the microbial consortium comprises at least Lactobacillus salivarius ACS-116-V-Col5a. Lactobacillus salivarius ACS-116-V-Col5a may be denoted by Taxonomy ID: 768728. The DNA sequence of Lactobacillus salivarius ACS-116-V-Col5a is provided by GenBank accession No. AEBA00000000.1. The 16S rRNA sequence of Lactobacillus salivarius ACS-116-V-Col5a is denoted as SEQ ID NO:24.

In some embodiments, the microbial consortium comprises at least Dialister succinatiphilus YIT 11850. Dialister succinatiphilus YIT 11850 may be denoted by Taxonomy ID: 742743. The DNA sequence of Dialister succinatiphilus YIT 11850 is provided by GenBank accession No. ADLT00000000.1. The 16S rRNA sequence of Dialister succinatiphilus YIT 11850 is denoted as SEQ ID NO:25.

In some embodiments, the microbial consortium comprises at least Bifidobacterium dentium ATCC 27678. Bifidobacterium dentium ATCC 27678 may be denoted by Taxonomy ID: 473819. The DNA sequence of Bifidobacterium dentium ATCC 27678 is provided by GenBank accession No. ABIX00000000.2. The 16S rRNA sequence of Bifidobacterium dentium ATCC 27678 is denoted as SEQ ID NO:26.

In some embodiments, the microbial consortium comprises at least Bifidobacterium dentium Bd1. Bifidobacterium dentium Bd1 may be denoted by Taxonomy ID: 401473. The DNA sequence of Bifidobacterium dentium Bd1 is provided by GenBank accession No. CP001750.1. The 16S rRNA sequence of Bifidobacterium dentium Bd1 is denoted as SEQ ID NO:27.

In some embodiments, the microbial consortium comprises at least Bifidobacterium adolescentis L2-32. Bifidobacterium adolescentis L2-32 may be denoted by Taxonomy ID: 411481. The DNA sequence of Bifidobacterium L2-32 is provided by GenBank accession No. AAXD00000000.2. The 16S rRNA sequence of Bifidobacterium L2-32 is denoted as SEQ ID NO:28.

In some embodiments, the microbial consortium comprises at least Bifidobacterium adolescentis 22L. Bifidobacterium adolescentis 22L may be denoted by assembly number GCF_000737885.1. The DNA sequence of Bifidobacterium adolescentis 22L is provided by GenBank accession No. CP007443.1. The 16S rRNA sequence of Bifidobacterium adolescentis 22L is denoted as SEQ ID NO:29.

In some embodiments, the microbial consortium comprises at least Mitsuokella multacida DSM 20544. Mitsuokella multacida DSM 20544 may be denoted by Taxonomy ID: 500635. The DNA sequence of Mitsuokella multacida DSM 20544 is provided by GenBank accession No. ABWK00000000.2. The 16S rRNA sequence of Mitsuokella multacida DSM 20544 is denoted as SEQ ID NO:30.

In some embodiments, the microbial consortium comprises at least Selenomonas sputigena ATCC 35185. Selenomonas sputigena ATCC 35185 may be denoted by Taxonomy ID: 546271. The DNA sequence of Selenomonas sputigena ATCC 35185 is provided by GenBank accession No. CP002637.1. The 16S rRNA sequence of Selenomonas sputigena ATCC 35185 is denoted as SEQ ID NO:31.

In some embodiments, the microbial consortium comprises at least Phascolarctobacterium succinatutens DSM 22533. Phascolarctobacterium succinatutens DSM 22533 may be denoted by Taxonomy ID: 626939. The DNA sequence of Phascolarctobacterium succinatutens DSM 22533 is provided by GenBank accession No. AEVN00000000.1. The 16S rRNA sequence of Phascolarctobacterium succinatutens DSM 22533 is denoted as SEQ ID NO:32.

In some embodiments, the microbial consortium comprises at least Phascolarctobacterium faecium JCM 30894. Phascolarctobacterium faecium JCM 30894 may be denoted by assembly number GCF_003945365.1. The DNA sequence of Phascolarctobacterium faecium JCM 30894 is provided by GenBank accession No. AP019004.1. The 16S rRNA sequence of Phascolarctobacterium faecium JCM 30894 is denoted as SEQ ID NO:33.

In some embodiments, the microbial consortium comprises at least Oscillibacter sp. PEA192. Oscillibacter sp. PEA192 may be denoted by Taxonomy ID: 2109687. The DNA sequence of Oscillibacter sp. PEA192 is provided by GenBank accession No. AP018532.1. The 16S rRNA sequence of Oscillibacter sp. PEA192 is denoted as SEQ ID NO:34.

In some embodiments, the microbial consortium comprises at least Eubacterium callanderi KIST612. Eubacterium callanderi KIST612 may be denoted by assembly number GCF_000152245.2. The DNA sequence of Eubacterium callanderi KIST612 is provided by GenBank accession No. CP002273.2. The 16S rRNA sequence of Eubacterium callanderi KIST612 is denoted as SEQ ID NO:35.

In some embodiments, the microbial consortium comprises at least Acetobacterium woodii DSM 1030. Acetobacterium woodii DSM 1030 may be denoted by Taxonomy ID: 931626. The DNA sequence of Acetobacterium woodii DSM 1030 is provided by GenBank accession No. CP002987.1. The 16S rRNA sequence of Acetobacterium woodii DSM 1030 is denoted as SEQ ID NO:36.

In some embodiments, the microbial consortium comprises at least Blautia producta PMF1. Blautia producta PMF1 may be denoted by assembly number GCF_004210255.1. The DNA sequence of Blautia producta PMF1 is provided by GenBank accession No. CP035945. The 16S rRNA sequence of Blautia producta PMF1 is denoted as SEQ ID NO:37.

In some embodiments, the microbial consortium comprises at least Eubacterium callanderi DSM 3662. Eubacterium callanderi DSM 3662 may be denoted by assembly number GCF_900142645.1. The DNA sequence of Eubacterium callanderi DSM 3662 is provided by GenBank accession No. FRBP00000000.1. The 16S rRNA sequence of Eubacterium callanderi DSM 3662 is denoted as SEQ ID NO:38.

In some embodiments, the microbial consortium comprises at least Eubacterium limosum SA11. Eubacterium limosum SA11 may be denoted by assembly number GCF_001481725.11. The DNA sequence of Eubacterium limosum SA11 is provided by GenBank accession No. CP011914.1. The 16S rRNA sequence of Eubacterium limosum SA11 is denoted as SEQ ID NO:39.

In some embodiments, the microbial consortium comprises at least Blautia hydrogenotrophica DSM 10507. Blautia hydrogenotrophica DSM 10507 may be denoted by Taxonomy ID: 476272. The DNA sequence of Blautia hydrogenotrophica DSM 10507 is provided by GenBank accession No. ACBZ00000000.1. The 16S rRNA sequence of Blautia hydrogenotrophica DSM 10507 is denoted as SEQ ID NO:40.

In some embodiments, at least one of the two or more microorganisms in the microbial consortium comprises 16S rRNA sequences denoted by SEQ ID NO:1.

In some other embodiments, at least one of the two or more microorganisms in the microbial consortium comprises 16S rRNA sequences denoted by SEQ ID NO:2.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 or SEQ ID NO:40.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4 or SEQ ID NO:5.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4 SEQ ID NO:5 or SEQ ID NO:8.

In some embodiments, at least one, at times at least two of the two or more microorganisms in the microbial consortium comprises 16S rRNA sequences denoted by SEQ ID NO:1, SEQ ID NO:2, SEQ ID NO:6, SEQ ID NO:7 or SEQ ID NO:8.

In some embodiments, at least one, at times at least two of the two or more microorganisms in the microbial consortium comprises 16S rRNA sequences denoted by SEQ ID NO:1, SEQ ID NO:3, SEQ ID NO:6, or SEQ ID NO:9.

In some embodiments, at least one, at times at least two of the two or more microorganisms in the microbial consortium comprises 16S rRNA sequences denoted by SEQ ID NO:1, SEQ ID NO:3, SEQ ID NO:6, SEQ ID NO:8 or SEQ ID NO:9.

In some embodiments, at least one, at times at least two of the two or more microorganisms in the microbial consortium comprises 16S rRNA sequences denoted by SEQ ID NO:1, SEQ ID NO:2, SEQ ID NO:4, SEQ ID NO:8 or SEQ ID NO:9.

In some embodiments, at least one, at times at least two of the two or more microorganisms in the microbial consortium comprises 16S rRNA sequences denoted by SEQ ID NO:1 or SEQ ID NO:2.

In some embodiments, at least one, at times at least two of the two or more microorganisms in the microbial consortium comprises 16S rRNA sequences denoted by SEQ ID NO:1, SEQ ID NO:2, SEQ ID NO:25, SEQ ID NO:4 or SEQ ID NO:5.

In some embodiments, at least one, at times at least two of the two or more microorganisms in the microbial consortium comprises 16S rRNA sequences denoted by SEQ ID NO:1, SEQ ID NO:25, SEQ ID NO:6 or SEQ ID NO:9.

In some embodiments, at least one, at times at least two of the two or more microorganisms in the microbial consortium comprises 16S rRNA sequences denoted by SEQ ID NO:1, SEQ ID NO:2 or SEQ ID NO:8.

In some embodiments, at least one, at times at least two of the two or more microorganisms in the microbial consortium comprises 16S rRNA sequences denoted by SEQ ID NO:1, SEQ ID NO:2, SEQ ID NO:25, SEQ ID NO:4, SEQ ID NO:5 or SEQ ID NO:8.

In some embodiments, at least one, at times at least two of the two or more microorganisms in the microbial consortium comprises 16S rRNA sequences denoted by SEQ ID NO:1, SEQ ID NO:25, SEQ ID NO:6, SEQ ID NO:8 or SEQ ID NO:9.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9, preferably SEQ ID NO:8.

In some embodiments, at least one microorganism in the microbial consortium comprises 16S rRNA sequences denoted by SEQ ID NO:8 and at least one other (different) microorganism in the microbial consortium comprises 16S rRNA sequences denoted by SEQ ID NO:1, SEQ ID NO:2, SEQ ID NO:3, SEQ ID NO:4, SEQ ID NO:5, SEQ ID NO:6, SEQ ID NO:7, or SEQ ID NO:9.

In some embodiments, the microbial consortium comprises at least one, at least two or more microorganisms selected from Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486, Megasphaera elsdenii ATCC 25940, Megasphaera sp. MJR8396C, Megasphaera hexanoica JCM 31403, Eubacterium hallii DSM 3353, Acetoanaerobium sticklandii DSM 519, Ruminococcaceae bacterium CPB6, Clostridium kluyveri DSM 555, Coprococcus comes ATCC 27758, Coprococcus catus GD/7, Roseburia inulinivorans DSM 16841, Lactobacillus ruminis ATCC 27782, Lactobacillus rhamnosus GG, Lactobacillus reuteri ATCC 55730, Lactobacillus plantarum subsp. plantarum LB1-2, Lactobacillus salivarius ACS-116-V-Col5a, Dialister succinatiphilus YIT 11850, Bifidobacterium dentium ATCC 27678, Bifidobacterium dentium Bd1, Bifidobacterium adolescentis L2-32, Bifidobacterium adolescentis 22L, Mitsuokella multacida DSM 20544, Selenomonas sputigena ATCC 35185, Phascolarctobacterium succinatutens DSM 22533, Phascolarctobacterium faecium JCM 30894, Oscillibacter sp. PEA192, Eubacterium callanderi KIST612, Acetobacterium woodii DSM 1030, Blautia producta PMF1, Eubacterium callanderi DSM 3662, Eubacterium limosum SA11 or Blautia hydrogenotrophica DSM 10507.

In some embodiments, the microbial consortium comprises at least one, at least two microorganisms selected from Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486 or any any combinations thereof.

In some embodiments, at least one microorganism in the microbial consortium comprises Hafnia alvei ATCC 51873 and at least one other (different) microorganism in the microbial consortium comprises Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, or Eubacterium limosum ATCC 8486.

In some embodiments, the microbial consortium comprises at least two microorganisms selected from Megasphaera elsdenii ATCC 17753, Lactobacillus ruminis ATCC 25644, Dialister invisus DSM 15470, Bifidobacterium dentium ATCC 27679, Eubacterium siraeum DSM 15702 or any combinations thereof.

In some embodiments, the microbial consortium comprises at least two microorganisms selected from Megasphaera elsdenii ATCC 17753, Lactobacillus ruminis ATCC 25644, Dialister invisus DSM 15470, Bifidobacterium dentium ATCC 27679, Eubacterium siraeum DSM 15702, Hafnia alvei ATCC 51873 or any combinations thereof.

In some embodiments, the microbial consortium comprises at least two microorganisms selected from Megasphaera elsdenii ATCC 17753, Lactobacillus ruminis ATCC 25644, Megamonas funiformis YIT 11815, Oscillibacter valericigenes DSM 18026, Hafnia alvei ATCC 51873, or any combinations thereof.

In some embodiments, the microbial consortium comprises at least two microorganisms selected from Megasphaera elsdenii ATCC 17753, Dialister invisus DSM 15470, Megamonas funiformis YIT 11815, Eubacterium limosum ATCC 8486 or any combinations thereof.

In some embodiments, the microbial consortium comprises at least two microorganisms selected from Megasphaera elsdenii ATCC 17753, Dialister invisus DSM 15470, Megamonas funiformis YIT 11815, Eubacterium limosum ATCC 8486, Hafnia alvei ATCC 51873 or any combinations thereof.

In some embodiments, the microbial consortium comprises at least one, at least two microorganisms selected from Megasphaera elsdenii ATCC 17753, Lactobacillus ruminis ATCC 25644, Bifidobacterium dentium ATCC 27679, Hafnia alvei ATCC 51873, Eubacterium limosum ATCC 8486 or any combinations thereof.

In some embodiments, the microbial consortium comprises at least two microorganisms selected from Megasphaera elsdenii ATCC 17753 and Lactobacillus ruminis ATCC 25644.

In some embodiments, the microbial consortium comprises at least two microorganisms selected from Megasphaera elsdenii ATCC 17753, Hafnia alvei ATCC 51873 and Lactobacillus ruminis ATCC 25644.

In some embodiments, the microbial consortium comprises at least two microorganisms selected from Megasphaera elsdenii ATCC 17753, Lactobacillus ruminis ATCC 25644, Dialister succinatiphilus YIT 11850, Bifidobacterium dentium ATCC 27679, Eubacterium siraeum DSM 15702 or any combinations thereof.

In some embodiments, the microbial consortium comprises at least two microorganisms selected from Megasphaera elsdenii ATCC 17753, Lactobacillus ruminis ATCC 25644, Dialister succinatiphilus YIT 11850, Bifidobacterium dentium ATCC 27679, Eubacterium siraeum DSM 15702, Hafnia alvei ATCC 51873 or any combinations thereof.

In some embodiments, the microbial consortium comprises at least two microorganisms selected from Megasphaera elsdenii ATCC 17753, Dialister succinatiphilus YIT 11850, Megamonas funiformis YIT 11815, Eubacterium limosum ATCC 8486 or any combinations thereof.

In some embodiments, the microbial consortium comprises at least two microorganisms selected from Megasphaera elsdenii ATCC 17753, Dialister succinatiphilus YIT 11850, Megamonas funiformis YIT 11815, Eubacterium limosum ATCC 8486, Hafnia alvei ATCC 51873 or any combinations thereof.

In some embodiments, the microbial consortium comprises Megasphaera elsdenii ATCC 17753.

In some other embodiments, the microbial consortium comprises Lactobacillus ruminis ATCC 25644.

In some embodiments, the microbial consortium comprises Hafnia alvei ATCC 51873.

In some embodiments, the microbial consortium comprises two microorganisms, as identified above. In some embodiments, the microbial consortium comprises a combination of two or three microorganisms, as identified above. In some embodiments, the microbial consortium comprises a combination of two, three or four microorganisms, as identified above. In some embodiments, the microbial consortium comprises a combination of two, three, four, five, six or more microorganisms as identified above.

In some embodiments, the microbial consortium comprises at least two isolated or purified microorganisms belonging to the genus, species or strain identified by NCBI Taxonomy IDs selected from the group consisting of NCBI Taxonomy ID or assembly number: 906, 39948, 1678, 459786, 568, 158846, 1730, 1578, 33042, 156454, 841, 970, 52225, 33024, 33951, 572511, 1485, 2305133, 1386, 698776, 365348, 2767881, 36866, 907, 1623, 218538, 1689, 39492, 437897, 351091, 569, 1736, 2023260, 1675036, 39488, 1511, 1898205, 1534, 410072, 116085, 360807, 47715, 1598, 1590, 1624, 487173, 1680, 52226, 69823, 626940, 33025, 1945593, 53442, 33952, 33035, 53443, 2144175, 156456, 187326, 1622, 1397, 2161821, 28026, 29360, 114197, 365349, 469613, 1520, 1625, 1232428, 1410663, 525362, 592028, 871562, 428128, 742816, 693746, 1002364, 1736, 1064535, 1603888, GCF_003315775.1, 411469, 499177, GCF_002119605.1, 431943, 470146, 717962, 622312, 1069534, 568703, 491077, GCF_002906875.1, 768728, 742743, 473819, 401473, 411481, GCF_000737885.1, 500635, 546271, 626939, GCF_003945365.1, 2109687, GCF_000152245.2, 931626, GCF_004210255.1, GCF_900142645.1, GCF_001481725.11 and 476272.

In some embodiments, the microbial consortium comprises Megasphaera elsdenii ATCC 17753, Lactobacillus ruminis ATCC 25644, Dialister invisus DSM 15470, Bifidobacterium dentium ATCC 27679 and Eubacterium siraeum DSM 15702. This microbial consortium comprising the five listed strains, as purified or isolated bacteria, is referred herein at times as Consortium 1 (“Cons. #1).

In some embodiments, the microbial consortium consists of Megasphaera elsdenii ATCC 17753, Lactobacillus ruminis ATCC 25644, Dialister invisus DSM 15470, Bifidobacterium dentium ATCC 27679 and Eubacterium siraeum DSM 15702.

In some embodiments, the microbial consortium comprises Megasphaera elsdenii ATCC 17753, Lactobacillus ruminis ATCC 25644, Megamonas funiformis YIT 11815, Oscillibacter valericigenes DSM 18026 and Hafnia alvei ATCC 51873. This microbial consortium comprising the five listed strains, as purified or isolated bacteria, is referred herein at times as Consortium 2 (“Cons. #2).

In some embodiments, the microbial consortium consists of Megasphaera elsdenii ATCC 17753, Lactobacillus ruminis ATCC 25644, Megamonas funiformis YIT 11815, Oscillibacter valericigenes DSM 18026 and Hafnia alvei ATCC 51873.

In some embodiments, the microbial consortium comprises Megasphaera elsdenii ATCC 17753, Dialister invisus DSM 15470, Megamonas funiformis YIT 11815, and Eubacterium limosum ATCC 8486. This microbial consortium comprising the four listed strains, as purified or isolated bacteria, is referred herein at times as Consortium 3 (“Cons. 3 #).

In some embodiments, the microbial consortium consists Megasphaera elsdenii ATCC 17753, Dialister invisus DSM 15470, Megamonas funiformis YIT 11815, and Eubacterium limosum ATCC 8486.

In some embodiments, the microbial consortium comprises Megasphaera elsdenii ATCC 17753, Lactobacillus ruminis ATCC 25644, Bifidobacterium dentium ATCC 27679, Hafnia alvei ATCC 51873 and Eubacterium limosum ATCC 8486. This microbial consortium comprising the five listed strains, as purified or isolated bacteria, is referred herein at times as Consortium 4 (“Cons. 4 #).

In some embodiments, the microbial consortium consists of Megasphaera elsdenii ATCC 17753, Lactobacillus ruminis ATCC 25644, Bifidobacterium dentium ATCC 27679, Hafnia alvei ATCC 51873 and Eubacterium limosum ATCC 8486.

In some embodiments, the microbial consortium comprises Megasphaera elsdenii ATCC 17753 and Lactobacillus ruminis ATCC 25644. This microbial consortium comprising the two listed strains, as purified or isolated bacteria, is referred herein at times as Consortium 5 (“Cons. #5)

In some embodiments, the microbial consortium comprises Megasphaera elsdenii ATCC 17753, Lactobacillus ruminis ATCC 25644, Dialister succinatiphilus YIT 11850, Bifidobacterium dentium ATCC 27679 and Eubacterium siraeum DSM 15702. This microbial consortium comprising the five listed strains, as purified or isolated bacteria, is referred herein at times as Consortium 6 (“Cons. #6).

In some embodiments, the microbial consortium consists of Megasphaera elsdenii ATCC 17753, Lactobacillus ruminis ATCC 25644, Dialister succinatiphilus YIT 11850, Bifidobacterium dentium ATCC 27679 and Eubacterium siraeum DSM 15702.

In some embodiments, the microbial consortium comprises Megasphaera elsdenii ATCC 17753, Dialister succinatiphilus YIT 11850, Megamonas funiformis YIT 11815, and Eubacterium limosum ATCC 8486. This microbial consortium comprising the four listed strains, as purified or isolated bacteria, is referred herein at times as Consortium 7 (“Cons. 7 #).

In some embodiments, the microbial consortium consists Megasphaera elsdenii ATCC 17753, Dialister succinatiphilus YIT 11850, Megamonas funiformis YIT 11815, and Eubacterium limosum ATCC 8486.

In some embodiments, the microbial consortium comprises Megasphaera elsdenii ATCC 17753, Lactobacillus ruminis ATCC 25644, Dialister invisus DSM 15470, Bifidobacterium dentium ATCC 27679, Hafnia alvei ATCC 51873 and Eubacterium siraeum DSM 15702. This microbial consortium comprising the six listed strains, as purified or isolated bacteria, is referred herein at times as Consortium 8 (“Cons. #8).

In some embodiments, the microbial consortium consists of Megasphaera elsdenii ATCC 17753, Lactobacillus ruminis ATCC 25644, Dialister invisus DSM 15470, Bifidobacterium dentium ATCC 27679, Hafnia alvei ATCC 51873 and Eubacterium siraeum DSM 15702.

In some embodiments, the microbial consortium comprises Megasphaera elsdenii ATCC 17753, Dialister invisus DSM 15470, Megamonas funiformis YIT 11815, Hafnia alvei ATCC 51873 and Eubacterium limosum ATCC 8486. This microbial consortium comprising the five listed strains, as purified or isolated bacteria, is referred herein at times as Consortium 9 (“Cons. 9 #).

In some embodiments, the microbial consortium consists Megasphaera elsdenii ATCC 17753, Dialister invisus DSM 15470, Megamonas funiformis YIT 11815, Hafnia alvei ATCC 51873 and Eubacterium limosum ATCC 8486.

In some embodiments, the microbial consortium comprises Megasphaera elsdenii ATCC 17753, Hafnia alvei ATCC 51873 and Lactobacillus ruminis ATCC 25644. This microbial consortium comprising the three listed strains, as purified or isolated bacteria, is referred herein at times as Consortium 10 (“Cons. #10).

In some embodiments, the microbial consortium comprises Megasphaera elsdenii ATCC 17753, Lactobacillus ruminis ATCC 25644, Dialister succinatiphilus YIT 11850, Bifidobacterium dentium ATCC 27679, Hafnia alvei ATCC 51873 and Eubacterium siraeum DSM 15702. This microbial consortium comprising the six listed strains, as purified or isolated bacteria, is referred herein at times as Consortium 11 (“Cons. #11).

In some embodiments, the microbial consortium consists of Megasphaera elsdenii ATCC 17753, Lactobacillus ruminis ATCC 25644, Dialister succinatiphilus YIT 11850, Bifidobacterium dentium ATCC 27679, Hafnia alvei ATCC 51873 and Eubacterium siraeum DSM 15702.

In some embodiments, the microbial consortium comprises Megasphaera elsdenii ATCC 17753, Dialister succinatiphilus YIT 11850, Megamonas funiformis YIT 11815, Hafnia alvei ATCC 51873 and Eubacterium limosum ATCC 8486. This microbial consortium comprising the five listed strains, as purified or isolated bacteria, is referred herein at times as Consortium 7 (“Cons. 11 #).

In some embodiments, the microbial consortium consists Megasphaera elsdenii ATCC 17753, Dialister succinatiphilus YIT 11850, Megamonas funiformis YIT 11815, Hafnia alvei ATCC 51873 and Eubacterium limosum ATCC 8486.

In some embodiments, the microbial consortium comprises Megasphaera elsdenii ATCC 17753, Dialister invisus DSM 15470, Megamonas funiformis YIT 11815, Eubacterium limosum ATCC 8486, Lactobacillus ruminis ATCC 25644, Bifidobacterium dentium ATCC 27679 and Hafnia alvei ATCC 51873. This microbial consortium comprising the seven listed strains, as purified or isolated bacteria, is referred herein at times as Consortium 12 (“Cons. #12).

In some embodiments, the microbial consortium consists Megasphaera elsdenii ATCC 17753, Dialister invisus DSM 15470, Megamonas funiformis YIT 11815, Eubacterium limosum ATCC 8486, Lactobacillus ruminis ATCC 25644, Bifidobacterium dentium ATCC 27679 and Hafnia alvei ATCC 51873.

In another aspect of the invention, which may be implemented as certain embodiments of the microbial consortium of the invention, provided it is a microbial consortium selected from the group consisting of Consortium 1, Consortium 2, Consortium 3, Consortium 4, Consortium 5, Consortium 6, Consortium 7, Consortium 8, Consortium 9, Consortium 10, Consortium 11 and Consortium 12.

In another aspect of the invention, which may be implemented as certain embodiments of the microbial consortium of the invention, provided it is a microbial consortium selected from the group consisting of Consortium 1, Consortium 2, Consortium 3, Consortium 4, Consortium 5, Consortium 6, Consortium 7 and Consortium 12.

In another aspect of the invention, which may be implemented as certain embodiments of the microbial consortium of the invention, provided it is a microbial consortium selected from the group consisting of Consortium 1, Consortium 2, Consortium 3, Consortium 4 and Consortium 12.

In another aspect of the invention, which may be implemented as certain embodiments of the microbial consortium of the invention, provided is a microbial consortium selected from Consortium 1 and Consortium 2.

In another aspect of the invention, which may be implemented as certain embodiments of the microbial consortium of the invention, provided is a microbial consortium selected from Consortium 3 and Consortium 4.

In another aspect of the invention, which may be implemented as certain embodiments of the microbial consortium of the invention, provided is a microbial consortium selected from Consortium 3, Consortium 4 and Consortium 12.

In some embodiments, the microbial consortium comprises same or equivalent amounts of the microorganisms forming the consortium.

In some further embodiments, the microbial consortium comprises different amounts of at least one of the microorganisms forming the consortium.

In some embodiments, the total number per one dose (cell count/amount/colony forming units-CFUs/Optical density measurement) of each microorganism forming the microbial consortium is at least 1×10², at least 1×10³, at least 10⁴, at least 1×10⁵, at times at least 1×10⁶, at times at least 1×10⁷, at times at least 1×10⁸.

In some embodiments, the total number per one dose (cell count/amount/colony forming units-CFUs/Optical density measurement) of each microorganism forming the microbial consortium between about 1×10⁸ and about 5×10¹⁰ CFU, between about 2×10⁸ and about 4×10¹⁰, between about 3×10⁸ and about 3×10¹⁰, between about 5×10⁸ and about 1×10¹⁰.

In some embodiments, the total number (cell count/amount/colony forming units-CFUs) of each one of microorganisms forming the microbial consortium is between about 1×10³ to about 1×10², between 1×10⁵ to about 1×10¹⁰, 1×10⁸ to about 5×10¹⁰, between 2×10⁸ to about 4×10¹⁰, between 3×10⁸ to about 3×10¹⁰, between 5×10⁸ to about 1×10¹⁰.

In some embodiments, the total number (cell count/amount/colony forming units-CFUs) of microorganisms forming the microbial consortium is between about 1×10³ to about 1×10², between 1×10⁵ to about 1×10¹⁰, 1×10⁸ to about 5×10¹⁰, between 2×10⁸ to about 4×10¹⁰, between 3×10⁸ to about 3×10¹⁰, between 5×10⁸ to about 1×10¹⁰.

The two or more microorganisms can be obtained from any available source. As detailed above, the two or more microorganisms can be identified in, purified or isolated from the microbiome of a reference subject, for example by collecting a biological sample. The biological sample may be any sample from which the population of microorganisms can be isolated, for example, feces. In yet another embodiment, the sample may be a biopsy of human organs or tissue, specifically, a gut biopsy.

The microbial consortium may be formulated in a variety of forms, depending on the storage, administration etc. Non-limiting forms include solid, dry form, for example, in a lyophilized powder, gel form, a suspension, a cell lysate or extract. In some embodiments, the microbial consortium may be suspended in a liquid medium (such as PBS or saline) and used in a suspension form.

The microbial consortium of the invention may be used in the preparation of a pharmaceutical formulation/compositions/suspension or in the manufacture of a formulation/compositions/suspension for use in treatment.

As such, formulation/compositions/suspension of the invention may comprise apart from a therapeutically effective amount of a microbial consortium of the invention, at least one additional component as detailed herein.

In a further aspect, the invention relates to a composition (suspension or formulation) comprising the microbial consortium of the invention.

As described herein, the microbial consortium and/or the suspension/composition comprising the same may form a kit of the invention. In general, the composition and/or the suspension and/or kit described herein comprising the microbial consortium as well as the microbial consortium per se form part of this invention. It should be noted that the forms described herein for the microbial consortium per se apply for the composition and/or the suspension and/or kit comprising the microbial consortium.

In yet some further embodiments, the composition of the invention may optionally further comprise at least one of pharmaceutically acceptable carrier/s, excipient/s, additive/s diluent/s and adjuvant/s. As used herein “pharmaceutically acceptable carrier” includes any and all solvents, dispersion media, coatings and the like.

Aqueous suspensions may further contain substances which increase the viscosity of the suspension including, for example, sodium carboxymethylcellulose, sorbitol and/or dextran. The suspension may also contain stabilizers.

It should be understood that in addition to the ingredients particularly mentioned above, the formulations may also include other agents conventional in the art having regard to the type of formulation in question.

The microbial consortium of the invention and/or any suspensions/compositions comprising the same can be administered and dosed by the methods of the invention as described below, in accordance with medical procedures known in the art. For example, the suspensions/compositions used in the methods and kits of the invention, described herein below, may be adapted for administration by various modes of administration that are known in the art including, for example, systemic, parenteral, intraperitoneal, transdermal, oral (including buccal or sublingual), rectal, topical (including buccal or sublingual), vaginal, intranasal and any other appropriate routes. Specific examples include but not limited to, injection (e.g., using a subcutaneous, intramuscular, intravenous, or intradermal injection), intranasal administration and oral administration.

In some embodiments, the microbial consortium of the invention and/or any suspensions/compositions comprising the same may be formulated for oral administration.

In some embodiments, the microbial consortium of the invention and/or any suspensions/compositions comprising the same may be formulated for delivery to the intestine. In some embodiments, the microbial consortium of the invention and/or any suspensions/compositions comprising the same may be formulated into food or a beverage.

In some embodiments, the microbial consortium of the invention and/or any suspensions/compositions comprising the same may be formulated to be contained within a carrier.

In some embodiments, the microbial consortium of the invention and/or any suspensions/compositions comprising the same is enterically coated.

As detailed herein and shown in the Examples below, it was surprisingly found that the microbial consortium comprises two or more microorganisms which are capable of modulating a variety of functions.

For example, as shown in Example 1A, it was found that Megasphaera elsdenii ATCC 17753 is capable of producing valerate and isovalerate.

In some examples, Megasphaera elsdenii ATCC 17753 produces valerate. In some examples, Megasphaera elsdenii ATCC 17753 produces isovalerate.

Further, as shown in Example 1A, Megasphaera elsdenii ATCC 17753 is capable of producing acetate, propionate and butyrate.

In some examples, Megasphaera elsdenii ATCC 17753 produces acetate. In some examples, Megasphaera elsdenii ATCC 17753 produces propionate. In some examples, Megasphaera elsdenii ATCC 17753 produces butyrate.

Further and as shown in the Example 1B, Megamonas funiformis YIT 11815 is capable of producing acetate and propionate.

In some examples, Megamonas funiformis YIT 11815 produces acetate. In some examples, Megamonas funiformis YIT 11815 produces propionate.

As shown in the Examples 1C and 1D, Bifidobacterium dentium ATCC 27679 and Hafnia alvei ATCC 51873, respectively are capable of producing acetate.

In some examples, Bifidobacterium dentium ATCC 27679 produces acetate. In some examples, Hafnia alvei ATCC 51873 produces acetate.

As shown in the Example 1E, Eubacterium limosum ATCC 8486 is capable of producing acetate and butyrate.

In some examples, Eubacterium limosum ATCC 8486produces acetate. In some examples, Eubacterium limosum ATCC 8486 produces butyrate.

As shown in the Example 1F, Bifidobacterium dentium ATCC 27679 and Eubacterium limosum ATCC 8486 are capable of producing GABA.

In some examples, Bifidobacterium dentium ATCC 27679 produces GABA. In some examples, Eubacterium limosum ATCC 8486 produces GABA.

As shown in the Example 11, Bifidobacterium dentium ATCC 27679 is capable of inducing serotonin secretion from RIN14B chromaffin cells from rat pancreas.

In some examples, Bifidobacterium dentium ATCC 27679 induces serotonine production. In some examples, Bifidobacterium dentium ATCC 27679 induces serotonine secretion.

As shown in the Example 1J, Hafnia alvei ATCC 51873 is capable of producing progesterone. In some examples, Hafnia alvei ATCC 51873 produced progesterone.

In some examples, Hafnia alvei ATCC 51873 degrades nitric oxide.

In some examples, Bifidobacterium dentium ATCC 27679 produces GABA.

In some examples, Eubacterium limosum ATCC 8486 produces GABA.

Hence, in accordance with some aspects, it is provided a microbial consortium comprising two or more microorganisms as described herein, for use in at least one of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In some embodiments, Megasphaera elsdenii ATCC 17753 is for use in modulating valerate and/or isovalerate levels. In some embodiments, Megasphaera elsdenii ATCC 17753 is for use in producing valerate. In some examples, the microbial consortium comprising Megasphaera elsdenii ATCC 17753 for use in a method of producing isovalerate.

In some embodiments, Megasphaera elsdenii ATCC 17753 is for use in modulating one or more of acetate, propionate, butyrate levels. In some embodiments, Megasphaera elsdenii ATCC 17753 is for use in producing one or more of acetate, propionate, butyrate.

In some embodiments, Megamonas funiformis YIT 11815 is for use in modulating one or more of acetate, propionate levels. In some embodiments, Megasphaera elsdenii ATCC 17753 is for use in producing one or more of acetate, propionate.

In some embodiments, at least one of Bifidobacterium dentium ATCC 27679, Hafnia alvei ATCC 51873 or any combinations thereof is for use in modulating acetate levels.

In some embodiments, at least one of Bifidobacterium dentium ATCC 27679, Hafnia alvei ATCC 51873 or any combinations thereof is for use in producing acetate.

In some embodiments, at least one of Bifidobacterium dentium ATCC 27679, Eubacterium limosum ATCC 8486 or any combinations thereof is for use in modulating GABA levels. In some embodiments, at least one of Bifidobacterium dentium ATCC 27679, Eubacterium limosum ATCC 8486 or any combinations thereof is for use in producing GABA.

In some embodiments, Bifidobacterium dentium ATCC 27679 is for use in modulating serotonin levels. In some embodiments, Bifidobacterium dentium ATCC 27679 is for use in inducing serotonin secretion.

In some embodiments, Hafnia alvei ATCC 51873 is for use in producing progesterone.

Based on the above, it was suggested that the microbial consortium of the present disclosure, the compositions comprising the microbial consortium of the present disclosure and the kits of the disclosure may be useful for a variety of purposes, including for treatment of subjects in need of a treatment by the identified microorganisms as well as the microbial consortium of the disclosure. Specifically, the identified microorganisms as well as the microbial consortium of the disclosure may be for use to treat a disorder treatable by the identified microorganisms as well as the microbial consortium and more specifically a disorder affect by modulation of any of the above.

In accordance with some aspects, it is provided a microbial consortium comprising two or more microorganisms, for use in treating a disorder being modulated/affected by at least one of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

In some embodiments, the types of the at least two microorganisms and the amount of each one of the at least two microorganisms in the microbial consortium, may be adapted based on the disorder to be treated or the severity of the disorder. For example, prior to treatment, diagnostics of the disorder and the severity may determine these parameters.

The disease or disorder as well as associated consideration as described herein are affected by modulating at least one of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide reduction, (xxv) estrogen production, (xxvi) progesterone production (xxvii) isoflavonoids metabolism, (xxviii) increase serotonin levels or (xxix) any combinations thereof.

In accordance with some aspects, it is provided the microbial consortiums as described herein for use in treating a disease or disorder, wherein the disease or disorder is affected by modulating at least one of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) iron sequestering, (xvii) secondary bile acid production, (xviii) pollutants detoxification, (xix) decreasing of methanogenesis, (xx) decreasing sulphate reduction, (xxi) Treg induction, (xxii) ammonia production, (xxiii) nitrite production, (xxiv) nitric oxide reduction, (xxv) estrogen production, (xxvi) progesterone production (xxvii) isoflavonoids metabolism, (xxviii) increase serotonin levels or (xxix) any combinations thereof.

In accordance with some other embodiments, the microbial consortium described herein, the compositions comprising the same or kits comprising the same may be useful in affecting the gastrointestinal system, the ENS or the CNS of a host subject and hence treat a subject being diagnosed with a functional gastrointestinal disorder.

Thus, the present invention further provides a microbial consortium as detailed above for use in the treatment of a subject suffering from a functional gastrointestinal disorder.

In some other embodiments, the microbial consortium is for use in treating a subject being diagnosed with a disorder that may be treatable by the consortium, such as a functional gastrointestinal disorder.

Thus, the present invention further provides a microbial consortium according to the invention comprising two or more purified or isolated microorganisms of the invention, the compositions comprising the same or kits of the invention for use in a method of treating of a subject suffering from a functional gastrointestinal disorder.

It was suggested that the microbial consortium of the invention can be used in method of treating a functional gastrointestinal disorder.

As used herein, a functional gastrointestinal disorder refers to any disorder or pathologically condition which affect different parts of the gastrointestinal tract and involve visceral hypersensitivity and motility disturbances.

The functional gastrointestinal disorder is considered in accordance with some embodiments, as having combination of the following: motility disturbance, visceral hypersensitivity, altered mucosal and immune function, altered gut microbiota, altered ENS processing and altered central nervous system (CNS) processing. It is of note that the functional gastrointestinal disorder encompasses related symptoms such as pain and bloating.

In some embodiments, the functional gastrointestinal disorder is a functional dyspepsia (FD). Functional dyspepsia is associated with one or more symptoms of bloating, burning, pain, or early fullness or prolonged fullness in the upper digestive tract, nausea, vomiting, burping, weight loss, sour taste in the mouth, that are present for at least one month.

In some embodiments, the functional dyspepsia is at least one of postprandial distress syndrome (PDS) or epigastric pain syndrome (EPS).

In some embodiments, the functional gastrointestinal disorder is a centrally mediated disorders of gastrointestinal pain.

In some embodiments, the centrally mediated disorders of gastrointestinal pain is centrally mediated abdominal pain syndrome (CAPS), narcotic bowel syndrome (NBS)/opioid-induced GI hyperalgesia or combination thereof.

Centrally mediated abdominal pain syndrome (CAPS), is a condition of abdominal (belly) pain that is long-term or repeated pain (keeps coming back), which is not related to changes in bowel pattern—constipation and/or diarrhea.

Narcotic bowel syndrome (NBS) is a condition that is characterized by worsening abdominal pain in the context of escalating or continuous opioid therapy.

In some embodiments, the functional gastrointestinal disorder is a bowel disorder.

In some embodiments, the bowel disorder is at least one of irritable bowel syndrome (IBS), functional constipation, functional diarrhea, functional abdominal bloating/distension, unspecified functional bowel disorder, opioid-induced constipation or combination thereof.

In some embodiments, the functional gastrointestinal disorder is IBS.

IBS is characterized by a variety of symptoms including abdominal pain and changes in the frequency and consistency of bowel movements.

In some embodiments, the IBS is at least one of IBS with predominant constipation (IBS-C), IBS with predominant diarrhea (IBS-D), IBS with mixed bowel habits (IBS-M), IBS unclassified (IBS-U) or combination thereof.

There are number of in vivo models that have been developed for studying the different conditions of the IBS-related GI dysfunctions. Amongst these models, different kinds of stressors, for instance, psychological and physical stimuli appear to play critical roles in the development of IBS and the maintenance of the disorder. Additionally, models that implement feces transplantation of IBS patients into germ-free animals can also stimulate the developments of IBS phenotypes such as visceral hypersensitivity and abnormal bowel movement—for more details “Pathogenesis, Experimental Models and Contemporary Pharmacotherapy of Irritable Bowel Syndrome: Story About the Brain-Gut Axis” S. W. Tsang et al, the context of which is incorporated by reference.

In some embodiments, the microbial consortium comprising Megasphaera elsdenii ATCC 17753 for use in a method of producing isovalerate in a subject suffering from a functional gastrointestinal disorder, preferably IBS, and wherein the production of isovalerate leads to treatment, inhibition, reduction, or elimination of the disorder in the subject.

In some examples, the microbial consortium comprising Hafnia alvei ATCC 51873 for use in a method of producing acetate in a subject suffering from a functional gastrointestinal disorder, preferably IBS, and wherein the production of acetate leads to treatment, inhibition, reduction, or elimination of the disorder in the subject.

In some embodiments, the microbial consortium comprising Hafnia alvei ATCC 51873 for use in a method of producing progesterone in a subject suffering from a functional gastrointestinal disorder, preferably IBS, and wherein the production of progesterone leads to treatment, inhibition, reduction, or elimination of the disorder in the subject.

In some embodiments, the microbial consortium comprising Hafnia alvei ATCC 51873 for use in a method of decomposing/depredating nitric oxide in a subject suffering from a functional gastrointestinal disorder, preferably IBS, and wherein the decomposing/depredating nitric oxide leads to treatment, inhibition, reduction, or elimination of the disorder in the subject.

In some examples, the microbial consortium comprising Bifidobacterium dentium ATCC 27679 for use in a method of producing GABA in a subject suffering from a functional gastrointestinal disorder, preferably IBS, and wherein the production of GABA leads to treatment, inhibition, reduction, or elimination of the disorder in the subject.

In some examples, the microbial consortium comprising Eubacterium limosum ATCC 8486 for use in a method of producing GABA in a subject suffering from a functional gastrointestinal disorder, preferably IBS, and wherein the production of GABA leads to treatment, inhibition, reduction, or elimination of the disorder in the subject.

In accordance with some other aspects, the invention provides a method for treating a disorder in a subject in need thereof. In some embodiments, the method for treating, preventing, ameliorating, reducing or delaying the onset of a functional gastrointestinal disorder in a subject in need thereof comprising administering to such subject a therapeutically effective amount of a microbial consortium of the invention or any composition or kit comprising the same.

In accordance with the methods of the invention, administering the effective amount of a microbial consortium of the invention ameliorates one or more signs or symptoms of the functional gastrointestinal disorder and specifically visceral hypersensitivity or IBS. In other words, the methods of the invention are for treating disorders may be treatable with the microbial consortium of the invention.

In an another aspect, the invention provides a method of treating, preventing, ameliorating, reducing or delaying the onset of a functional gastrointestinal disorder, in a human subject in need thereof comprising the step of administering to the subject an effective amount of a microbial consortium comprising two or more isolated microorganism or purified microorganisms, at least one of the two or more microorganisms is having one or more of the following features: (i) capable of modulating visceral hypersensitivity, (ii) capable of modulating intraluminal gas balance, (iii) capable of modulating ENS, (iv) capable of modulating gut-brain axis, (v) capable of modulating GABA, (vi) capable of modulating gut-barrier, (vii) capable of modulating pathways associated with low-grade inflammation, (viii) capable of modulating fatty acid production, (ix) capable of modulating dietary compounds metabolism, (x) capable of modulating detox activity, (xi) capable of modulating an endopeptidase activity, (xii) capable of modulating serotonin or (xiii) any combinations thereof.

In some embodiments of the methods of the invention for treating, preventing, ameliorating, reducing or delaying the onset of IBS, comprising administrating to the subject in need thereof a therapeutically effective amount of the microbial consortium of the invention.

The methods of the invention comprise in accordance with some embodiments, administration of the microbial consortium of the invention with an additional treatment.

In some embodiments the microbial consortium may be administrated in combination with an additional treatment. The microbial consortium and the additional treatment or treatments may be administrated simultaneously or sequentially.

As appreciated, the additional treatment is selected, for example, to be compatible with the microbial consortium for the purpose of improving the effect of the microbial consortium. In some embodiments, the additional treatment is a pain relief treatment. In some embodiments, the additional treatment is a diarrhea treatment. In some embodiments, the additional treatment is a constipation treatment.

The microbial consortium may be administrated to a human subject by any method known in the art and described herein. In accordance with some embodiments, the methods of the invention comprise administration the microbial consortium or a composition comprising the microbial consortium by oral administration. In accordance with some embodiments, the methods of the invention comprise administration the microbial consortium or a composition comprising the microbial consortium that is formulated for delivery to the intestine. The microbial consortium or a composition comprising the microbial consortium may be formulated for example into a capsule, tablet, food or beverage or any of the like.

In some embodiments, the methods of the invention may comprise administrating the microbial consortium of the invention, compositions or kits comprising the same and optionally an additional treatment, as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month.

The invention further provides the use of a microbial consortium of the invention in the preparation of a composition for treating a functional gastrointestinal disorder, specifically IBS in a subject in need thereof.

In another aspect, the invention provides a kit comprising microbial consortium of the invention in the preparation of a composition for treating a functional gastrointestinal disorder, in a subject in need thereof.

In some embodiments, the kits described herein may include a composition/suspension as described, as an already prepared dosage form ready for administration or, alternatively, can include the microbial consortium or a composition comprising the microbial consortium as described as a solid pharmaceutical composition (e.g. in a lyophilized form) that can be reconstituted with a solvent to provide a liquid dosage form. The kit of the invention may additionally comprise instructions for using the kit in treating a functional gastrointestinal disorder, specifically IBS.

The methods of treatment provided herein involve administration of the microbial consortium and/or a composition comprising the same in a therapeutically effective amount. The term “effective amount” as used herein is that determined by such considerations as are known to the man of skill in the art. The amount must be sufficient to improve the disorder or conditions described herein. Dosing is dependent on the severity of the symptoms and on the responsiveness of the subject to at least one microorganism of the invention. Medically professionals can easily determine the optimum dosage, dosing methodology and repetition rates, taking into consideration the age, sex, weight and state of the disease of the subject to be treated, will determine the dose.

As use herein “therapeutically effective amount” means an amount of a microbial consortium and/or a composition comprising the same which provides a medical benefit as noted by the clinician or other qualified observer.

The methods of treatment provided herein involve administration of the microbial consortium of the invention in a therapeutically effective amount. The term “effective amount” as used herein is that determined by such considerations as are known to the man of skill in the art.

The present invention provides methods for treating or preventing a functional gastrointestinal disorder. The term “treatment or prevention” refers to the complete range of therapeutically positive effects of administrating to a subject including inhibition, reduction of, alleviation of, and relief from, a functional gastrointestinal disorder, or undesired side effects of such as a functional gastrointestinal disorder-related disorders.

As used herein, “disease”, “disorder”, “condition” and the like, as they relate to a subject's health, are used interchangeably and have meanings ascribed to each and all of such terms.

The present invention relates to methods for the treatment of subjects, or patients, in need thereof. By “patient” or “subject in need” it is meant any organism who may be affected by the above-mentioned conditions, and to whom the treatment methods herein described are desired. It should be further noted that particularly in case of human subject, administering of the compositions of the invention to the patient includes both self-administration and administration to the patient by another person.

The invention provides methods for treating a functional gastrointestinal disorder, and further relates to disorders associated or related to a functional gastrointestinal disorder. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

As noted that “modulation” as used herein encompasses either inhibition, decrease, lowering and the like, or alternatively, increase, enhance and the like.

More specifically, the terms “inhibition” “reduce” or “decrease” as referred to herein, relate to the retardation, restraining or lowering of a specified pathway/substance by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% as compared as compared to a suitable control.

Alternatively, the terms “increase” or “enhancement” as used herein relate to the act of becoming progressively greater in size, amount, content, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of a specific pathway/content as compared to a suitable control.

It should be noted that a suitable control refers at times to the respective pathway/substance without the microbial consortium.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It should be noted that the term “at least one” refers to one or more of the following and encompasses any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more of the specified information. For example, when referring to at least one microorganiam, it should be understood to refer to one or more microorganism and to encompasses any one of 1, 2, 3, 4, 5, 6, 7, 8, 9, 10, or more microorganisms.

It is to be understood that the terminology used herein is used for the purpose of describing particular embodiments only and not intended to be limiting since the scope of the present invention will be limited only by the appended claims and equivalents thereof.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It should be noted that the various embodiments and examples detailed herein in connection with various aspects of the invention may be applicable to one or more aspects disclosed herein. It should be further noted that any embodiment described herein, for example, related to components of the microbial consortium, may be applied separately or in various combinations. Various embodiments and aspects of the present invention as delineated hereinabove and as claimed in the claims section below find experimental support in the following examples. The phrases “in another embodiment” or any reference made to embodiment as used herein do not necessarily refer to different embodiment, although it may. Thus, various embodiments of the invention can be combined (from the same or from different aspects) without departing from the scope of the invention.

TABLE 1
List of Sequences

| SEQ ID | |
|---|---|
| NO: | Details |
| 1 | Gene encoding 16S rRNA of Megasphaera elsdenii ATCC 17753 |
| 2 | Gene encoding 16S rRNA of Lactobacillus ruminis ATCC 25644 |
| 3 | Gene encoding 16S rRNA of Dialister invisus DSM 15470 |
| 4 | Gene encoding 16S rRNA of Bifidobacterium dentium ATCC 27679 |
| 5 | Gene encoding 16S rRNA of Eubacterium siraeum DSM 15702 |
| 6 | Gene encoding 16S rRNA of Megamonas funiformis YIT 11815 |
| 7 | Gene encoding 16S rRNA of Oscillibacter valericigenes DSM 18026 |
| 8 | Gene encoding 16S rRNA of Hafnia alvei ATCC 51873 |
| 9 | Gene encoding 16S rRNA of Eubacterium limosum ATCC 8486 |
| 10 | Gene encoding 16S rRNA of Megasphaera elsdenii ATCC 25940 |
| 11 | Gene encoding 16S rRNA of Megasphaera sp. MJR8396C |
| 12 | Gene encoding 16S rRNA of Megasphaera hexanoica JCM 31403 |
| 13 | Gene encoding 16S rRNA of Eubacterium hallii DSM 3353 |
| 14 | Gene encoding 16S rRNA of Acetoanaerobium sticklandii DSM 519 |
| 15 | Gene encoding 16S rRNA of Ruminococcaceae bacterium CPB6 |
| 16 | Gene encoding 16S rRNA of Clostridium kluyveri DSM 555 |
| 17 | Gene encoding 16S rRNA of Coprococcus comes ATCC 27758 |
| 18 | Gene encoding 16S rRNA of Coprococcus catus GD/7 |
| 19 | Gene encoding 16S rRNA of Roseburia inulinivorans DSM 16841 |
| 20 | Gene encoding 16S rRNA of Lactobacillus ruminis ATCC 27782 |
| 21 | Gene encoding 16S rRNA of Lactobacillus rhamnosus GG |
| 22 | Gene encoding 16S rRNA of Lactobacillus reuteri ATCC 55730 |
| 23 | Gene encoding 16S rRNA of Lactobacillus plantarum subsp. plantarum |
| | LB1-2 |
| 24 | Gene encoding 16S rRNA of Lactobacillus salivarius ACS-116-V-Col5a |
| 25 | Gene encoding 16S rRNA of Dialister succinatiphilus YIT 11850 |
| 26 | Gene encoding 16S rRNA of Bifidobacterium dentium ATCC 27678 |
| 27 | Gene encoding 16S rRNA of Bifidobacterium dentium Bd1 |
| 28 | Gene encoding 16S rRNA of Bifidobacterium adolescentis L2-32 |
| 29 | Gene encoding 16S rRNA of Bifidobacterium adolescentis 22L |
| 30 | Gene encoding 16S rRNA of Mitsuokella multacida DSM 20544 |
| 31 | Gene encoding 16S rRNA of Selenomonas sputigena ATCC 35185 |
| 32 | Gene encoding 16S rRNA of Phascolarctobacterium succinatutens DSM |
| | 22533 |
| 33 | Gene encoding 16S rRNA of Phascolarctobacterium faecium JCM 30894 |
| 34 | Gene encoding 16S rRNA of Oscillibacter sp. PEA192 |
| 35 | Gene encoding 16S rRNA of Eubacterium callanderi KIST612 |
| 36 | Gene encoding 16S rRNA of Acetobacterium woodii DSM 1030 |
| 37 | Gene encoding 16S rRNA of Blautia producta PMF1 |
| 38 | Gene encoding 16S rRNA of Eubacterium callanderi DSM 3662 |
| 39 | Gene encoding 16S rRNA of Eubacterium limosum SA11 |
| 40 | Gene encoding 16S rRNA of Blautia hydrogenotrophica DSM 10507 |

### SOME NON-LIMITING EXAMPLES

### Example 1: In Vitro Assays

### Example 1A: SCFAs Production by Megasphaera elsdenii ATCC 17753

### Methods

Growth medium, either de Man, Rogosa and Sharpe (MRS) or MRS medium supplemented with 90 mM lactate was inoculated with 1% inoculum of overnight cultures of Megasphaera elsdenii ATCC 17753 (12 hours), followed by incubation anaerobically at 37° C. for up to 48 hours. Cells were removed by centrifugation (10 min, 3220 g, 4° C.), and filtered media was stored frozen at −80° C. until analysis. The detection of secreted SCFAs was performed using Gas Chromatography (GC) analysis. Results were expressed in μg/ml.

### Results

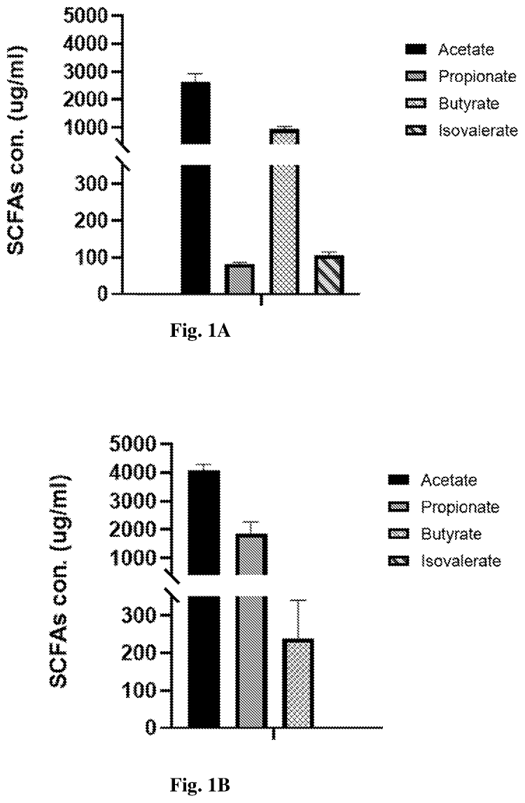

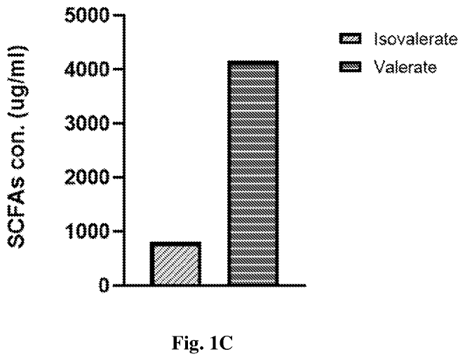

FIGS. 1A to 1C show results of SCFAs Production. FIGS. 1A and 1B show production of acetate, propionate, butyrate and isovalerate by Megasphaera elsdenii ATCC 17753 in MRS medium (FIG. 1A) or MRS medium with lactate (FIG. 1B). As can be seen, Megasphaera elsdenii ATCC 17753 produced isovalerate only in the absence of lactate after 7 hours of incubation. FIG. 1C shows production of isovalerate and valerate by Megasphaera elsdenii ATCC 17753 in MRS medium with lactate. As can be seen, Megasphaera elsdenii ATCC 17753 produced valerate and isovalerate in a medium comprising lactate when measured after 26 hours of incubation. As can be also seen, valerate was produced at higher concentrations as compared to isovalerate.

### Example 1B: SCFAs Production by Megamonas funiformis YIT 11815

### Methods

Growth medium, MRS, was inoculated with 1% inoculum of overnight cultures of Megamonas funiformis YIT 11815 (12 hours), followed by incubation anaerobically at 37° C. for up to 48 hours. Cells were removed by centrifugation (10 min, 3220 g, 4° C.), and filtered media was stored frozen at −80° C. until analysis. The detection of secreted SCFAs was performed using Gas Chromatography (GC) analysis. Results were expressed in μg/ml.

### Results

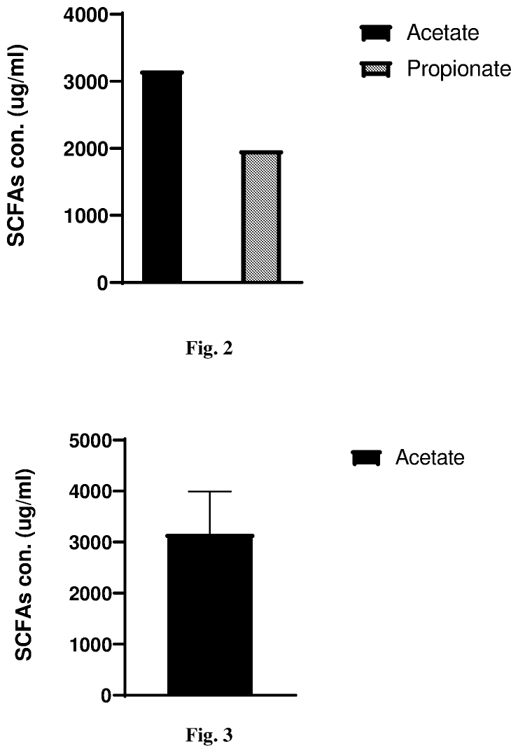

FIG. 2 shows results of acetate and propionate production by Megamonas funiformis YIT 11815. As can be seen, Megamonas funiformis YIT 11815 produced acetate and propionate after 30 hours of incubation.

### Example 1C: SCFAs Production by Bifidobacterium dentium ATCC 27679

### Methods

Growth medium, MRS, was inoculated with 1% inoculum of overnight cultures of Bifidobacterium dentium ATCC 27679 (12 hours), followed by incubation anaerobically at 37° C. for up to 48 hours. Cells were removed by centrifugation (10 min, 3220 g, 4° C.), and filtered media was stored frozen at −80° C. until analysis. The detection of secreted SCFAs was performed using Gas Chromatography (GC) analysis. Results were expressed in μg/ml.

### Results

FIG. 3 shows results of acetate production by Bifidobacterium dentium ATCC 27679. As can be seen, Bifidobacterium dentium ATCC 27679 produced acetate when measured after 26 hours of incubation.

### Example 1D: SCFAs Production by Hafnia alvei ATCC 51873

### Methods

Growth medium, Brain heart infusion (BHI), was inoculated with 1% inoculum of overnight cultures of Hafnia alvei ATCC 51873 (12 hours), followed by incubation anaerobically at 37° C. for up to 24 hours. Cells were removed by centrifugation (10 min, 3220 g, 4° C.), and filtered media was stored frozen at −80° C. until analysis. The detection of secreted SCFAs was performed using Gas Chromatography (GC) analysis. Results were expressed in μg/ml.

### Results

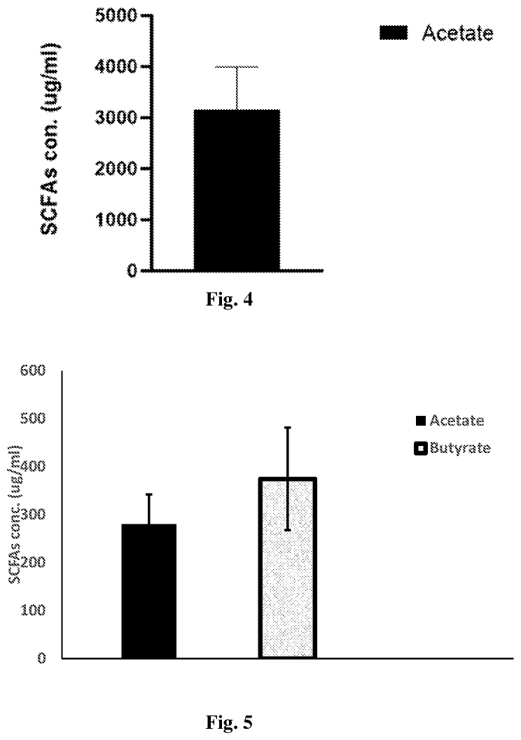

FIG. 4 shows results of acetate production by Hafnia alvei ATCC 51873. As can be seen, Hafnia alvei ATCC 51873 produced acetate when measured after 9.5 hours of incubation.

### Example 1E: SCFAs Production by Eubacterium limosum ATCC 8486

### Methods

Growth medium, FTM, was inoculated with 1% inoculum of overnight cultures of Eubacterium limosum ATCC 8486 (12 hours), followed by incubation anaerobically at 37° C. for up to 24 hours. Cells were removed by centrifugation (10 min, 3220 g, 4° C.), and filtered media was stored frozen at −80° C. until analysis. The detection of secreted SCFAs was performed using Gas Chromatography (GC) analysis. Results were expressed in μg/ml.

### Results

FIG. 5 shows results of acetate and butyrate production by Eubacterium limosum ATCC 8486.

### Example 1F: Bacterial GABA Production

Microorganisms were tested for their ability to produce and secrete GABA. Growth medium specific for each bacterium was inoculated with 1% inoculum of overnight cultures (12 hours), followed by incubation anaerobically at 37° C. for up to 72 hours. Cells were removed by centrifugation (10 min, 3220 g, 4° C.), filtration through syringe filters (13 mm diameter, 0.22 μm pore size) and filtered media was stored frozen at −80° C. until analysis. The detection of secreted GABA was performed using HPLC analysis and GABA ELISA.

### Methods

1% inoculum of overnight cultures of Bifidobacterium dentium ATCC 27679 (12 hours), Eubacterium limosum ATCC 8486, and Cetobacterium somerae ATCC BAA-474 as a negative control bacterium were used to inoculate MRS medium supplemented with 3% glutamate, Fluid Thioglycollate Medium (FTM) supplemented with 1% glutamate and

BHI medium supplemented with 30 mM putrescine, respectively and incubated anaerobically at 37° C. for up to 72 hours. Cells were removed by centrifugation (10 min, 3220 g, 4° C.), and filtered media was stored frozen at −80° C. until analysis. The detection of secreted GABA was performed using HPLC analysis. Results were expressed in μg/ml.

### Results

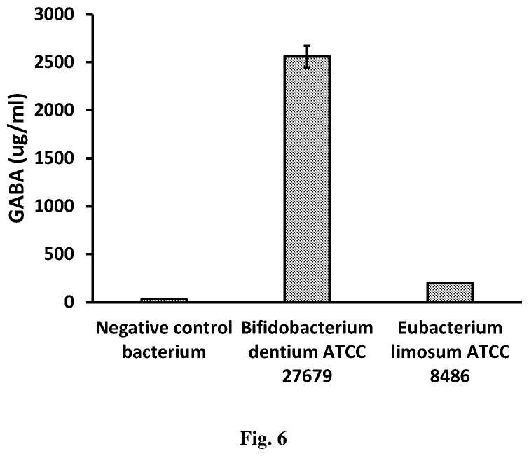

FIG. 6 shows results of GABA production by Bifidobacterium dentium ATCC 27679, Eubacterium limosum ATCC 8486 and Cetobacterium somerae ATCC BAA-474. As can be seen, Bifidobacterium dentium ATCC 27679 and Eubacterium limosum ATCC 8486 produce a significant amount of GABA compared to the negative control bacteria.

### Example 1G: In Vitro Gut System

Experiments are performed using the SHIME® system. Briefly, the system comprised of reactor vessels that simulate the conditions of the proximal colon and distal colon.

The bacterial consortia are added to all vessels and monitored over time for microbial activity parameters such as SCFAs composition, GABA levels, lactate levels, gas levels, pH, and ammonium levels. The vessels are also monitored for microbial load and microbial composition.

### Example 1H: In Vitro Progesterone Synthesis

In these experiments, the ability of Hafnia alvei ATCC 51873 to synthesize progesterone was tested by means of quantifying the expression of the progesterone producing gene 3β-hydroxysteroid dehydrogenase (HSDs).

Hafnia alvei ATCC 51873 cells were grown with and without the progesterone precursor, pregnenolone, over a course of 48 hours. Cells were harvested, lysed and RNA was extracted. Lysis and purification of RNA were performed according to RNA protect protocol for Enzymatic Lysis, and Proteinase K Digestion of bacteria followed by RNA purification using RNeasy Mini Kit (Qiagen, catalog no. 74104). As specified in this kit, Proteinase K from Tritirachium (Sigma, catalog no. P2308) was used for peptide digestion, and enzymatic lysis was carried out using Lysozyme from chicken egg white (Sigma, catalog no. L6876). Purification was completed with removal of excess DNA using RNase-Free Dnase Set (Qiagen, catalog no. 79254). Pure RNA was stored in −20° C. until cDNA library preparation. cDNA library was constructed using High Capacity cDNA Reverse Transcription Kit (Thermo Fisher Scientific, catalog no. 4368814). Real-time PCR was performed on StepOnePlus™ Real-Time PCR System (Applied Biosystems, 4376600), using PowerUp™ Syber Green™ mastermix (Applied Biosystems, A25742). RecA and Rho were used as housekeeping genes (HKGs) controls. Results were presented as fold change, calculated according to the acceptable method of 2\hat{ }(−ΔΔ Ct). ΔCt was calculated by first subtracting the average Ct value of the gene of interest from the housekeeping gene, RecA. ΔΔ Ct was then calculated by subtracting the Δ Ct value of the treated sample from the control samples that did not contain the precursor of progesterone, pregnenolone.

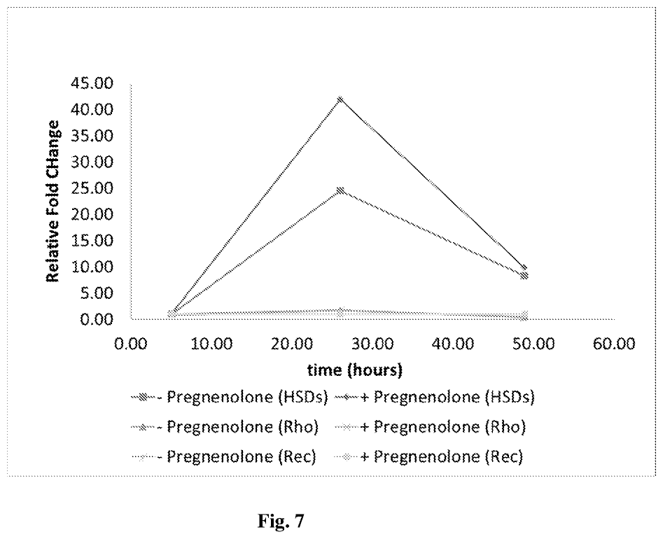

The results presented in FIG. 7 demonstrate that the enzyme transcripts of HSDs were increased along incubation time and reached a maximum after 24 hours of incubation. At that time, enzyme transcripts of HSDs were approximately two folds higher in samples originating from Hafnia alvei ATCC 51873 fermentation with pregnenolone as compared to Hafnia alvei ATCC 51873 fermentation without pregnenolone.

These results show that Hafnia alvei ATCC 51873 is potentially capable of synthesizing progesterone.

### Example 1I: In Vitro Serotonin Synthesis

In these experiments, the ability of Bifidobacterium dentium ATCC 27679 to induce serotonin secretion from RIN14B chromaffin cells from rat pancreas (ATCC CRL-2059) was tested. To test this, bacterial-cultured media were incubated with RIN14B cells for 1 hour.

RIN14B cells were plated on 96-well plates, at 3.5×10\hat{ }4 cells per well in 200 μL culture medium. The cells were let to attach to the plate for 16-24 hours at 37° C., 5% C02.

Then, media of all wells were discarded, and the cells were washed once with Hanks' Balanced Salt solution (HBSS) containing 0.2% BSA and 2 μM fluoxetine (by filling the wells with 200 μL solution and discarding immediately). Cells were incubated with 200 μL culture medium supplemented with 30 μM of the positive control ionomycin and cultured medium of Bifidobacterium dentium ATCC 27679, for 1 at 37° C., 5% C02. At the end of incubation time, supernatants of RIN14B cells were collected and Serotonin levels were measured by ELISA (EIA Serotonin, Beckmancoulter, catalogue number IM1749).

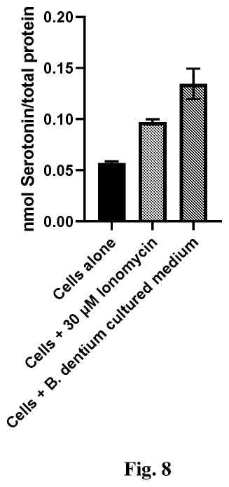

The results presented in FIG. 8 demonstrate that incubation of the Bifidobacterium dentium ATCC 27679 cultured-medium with RIN14B cells led to a higher serotonin secretion compared to RIN14B cells incubated with the positive control ionomycin and cells alone.

These results may suggest that compounds (metabolites) produced by Bifidobacterium dentium ATCC 27679, such as acetate and/or GABA and which are secreted to the medium can induce Serotonin secretion from RIN14B chromaffin cells.

### Example 1J: In Vitro Histamine Synthesis

This study is aimed to test the ability of bacteria to prevent release of histamine from mast cells in cell line RBL-2H3 (ATCC CRL-2256), which are mast cells analogues. Rat basophilic leukemia cells RBL-2H3 are plated in 96 well plates, at 5×10\hat{ }4 cells per well in 200 μL in culture medium. The cells are allowed to attach to the plate for 20-24 hours at 37° C., 5% C02. Then, medium of all wells is discarded and the cells are washed with PBS. Thereafter, cells are incubated with 200 μL culture medium supplemented with bacterial cultured media, for 1 or 3 hours at 37° C., 5% C02.

At the end of incubation period, cells are washed with culture medium and stimulated with culture medium containing monoclonal TNP-IgE and subsequently TNP-BSA at 37° C., 5% CO₂. Supernatants of RBL-2H3 cells are collected, and Histamine levels are measured by ELISA (HIS (histanime) ELISA Kit, Elabscience, catalogue number E-EL-0032).

The results showing reduction in Histamine level may suggest that compounds (metabolites) produced by different bacteria can prevent release of histamine from mast cells. Activation of mast cells in the human gut leads to histamine secretion, when this process is in proximity to colonic nerve it is correlated with abdominal pain as described in IBS.

### Example 2: In Vivo Studies

### Example 2A: The Wrap-Restraint Stress (WRS) Model

Rodents undergo a WRS-induced stress develop an immediate hyperalgesia, quantifiable in colon-rectal distention (CRD) frequency measurement. In addition, the stimulation of large intestinal activity is witnessed and followed by an increased fecal excretion.

In this study, the efficacy of bacterial consortia administration in ameliorating visceral hypersensitivity of WRS-induced stress in a rat model was tested.

Rats were administered with three exemplary bacterial consortia via oral gavage three time a week for three weeks prior to WRS-acute induced stress. 10\hat{ }9 CFU of each bacterium in a consortium was administered in each gavage. PBS+15% glycerol was administered similarly as control vehicle.

The three bacterial consortia were as follows: Consortium 3 included Megasphaera elsdenii ATCC 17753, Dialister invisus DSM 15470, Megamonas funiformis YIT 11815, and Eubacterium limosum ATCC 8486. Consortium 4 included Megasphaera elsdenii ATCC 17753, Lactobacillus ruminis ATCC 25644, Bifidobacterium dentium ATCC 27679, Hafnia alvei ATCC 51873 and Eubacterium limosum ATCC 8486. Consortium 12 included Megasphaera elsdenii ATCC 17753, Dialister invisus DSM 15470, Megamonas funiformis YIT 11815, Eubacterium limosum ATCC 8486, Lactobacillus ruminis ATCC 25644, Bifidobacterium dentium ATCC 27679 and Hafnia alvei ATCC 51873.

During the WRS test, excreted fecal pellets were counted for each animal in the different groups. Following WRS induction, a distension balloon mounted on a catheter filled with water and connected to a pressure transducer was introduced into the rectum. Balloon catheter was inserted into the distal colon with the distal tip 2 cm from the anal verge and secured to the base of the tail with surgical adhesive tape. To allow CRD and abdominal contractions (AC) recording, a pressure transducer was connected to both a syringe and an amplifier (TBM4M, World Precision Instruments). The balloon was then progressively inflated by intervals of 0.4 mL each 5 min from 0 to 1.2 mL. For each animal, the number of AC produced during each step of distension was recorded.

At the end of the experiment, the colon and the ileum of the animals were removed for histological analysis.

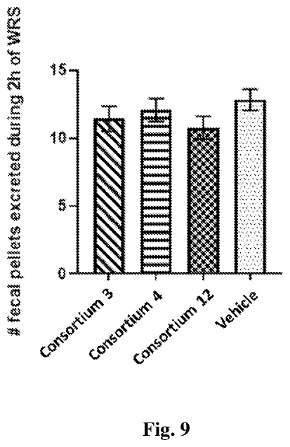

As can be seen in FIG. 9, all three bacterial treatments exhibited a decrease in the number of fecal pellets excreted during the 2 h of WRS.

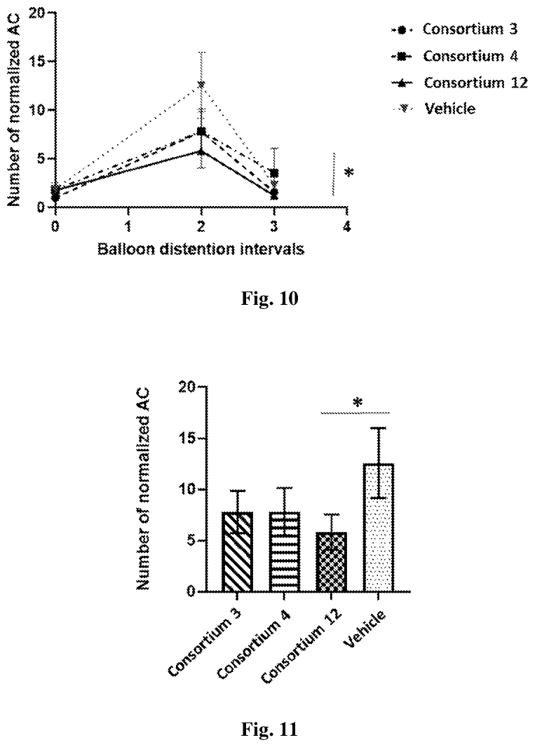

The number of normalized AC during the different inflated balloon volumes were lower in the treatments groups as opposed to the vehicle group (FIG. 10). The results show that consortium 12 exhibited a significantly lower AC during the different inflated balloon volumes when compared to the vehicle group. Moreover, this effect was observed significantly in balloon volume of 0.8 ml when comparing the treatments including treatment with consortium 12 and vehicle (FIG. 11).

### Example 2B: The Maternal Separation (MS) Model

The stress in this model is induced by removing the rat pups from their mother for 3 hours per day during the first two weeks of life. Since maternal care affects the hypothalamic-pituitary-adrenal (HPA) axis and the cognitive and emotional functions, the MS causes stable changes in the central nervous system of these animals. In adult animals, that have experienced this stress as pups, MS promotes the development of a condition in the large intestine characterized by visceral hypersensitivity to colorectal distension (CRD), in which colonic mast cell hyperplasia often concentrated close to nerve endings that are two typical signs of IBS. Additionally, colonic motor function in response to stress is also enhanced in these animals. CRD, total gastrointestinal transit and colonic motility are measured.

### Example 2C: Rat Model of Visceral Hypersensitivity Using Fecal Microbiota from IBS Human Subjects

Germ-free (GF) rats are kept in positive-pressure sterile isolators with free access to irradiated standard rodent diet and sterilized drinking water. Each GF rat is inoculated by an intragastric gavage with 2 mL of human fecal suspension in order to obtain Human Microbiota-Associated (HMA) rats.

In all in vivo models behavioral pain responses, visceral sensitivity and whole gut transit time are evaluated. In addition to these assays, the different SCFAs and GABA are quantified in the animals' feces according to the methods stated above.

1. A microbial consortium comprising two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

2. The microbial consortium of claim 1, having at least eight of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels or (xxix) any combinations thereof.

3-5. (canceled)

6. The microbial consortium of claim 1, wherein:
(a) at least one of the two or more microorganisms is capable of having at least five of (i) vitamin B12 production, (ii) GABA production (iii) valerate production, (iv) isovalerate production, (v) decreasing activity of at least one serine protease, (vi) increasing iron sequestering, (vii) caproate production, (viii) pollutants detoxification, (ix) linoleic acid production (x) acetate production, (xi) butyrate production, (xii) propionate production, (xiii) decreasing methanogenesis, (xiv) ammonia production, (xv) nitrite production, (xvi) nitric oxide degradation, (xvii) isoflavonoids metabolism (xviii) decreasing sulphate reduction, or (ixx) any combinations thereof;
(b) at least one of the two or more microorganisms is capable of having at least five of (i) vitamin B12 production, (ii) GABA production, (iii) valerate production, (iv) isovalerate production, (v) decreasing activity of at least one serine protease, (vi) increasing iron sequestering, (vii) caproate production, (viii) pollutants detoxification, (ix) linoleic acid production, (x) acetate production, (xi) butyrate production, (xii) propionate production, (xiii) decreasing methanogenesis, (xiv) ammonia production, (xv) nitrate production, (xvi) nitric oxide degradation, (xvii) isoflavonoids metabolism, or (xviii) any combinations thereof; or
(c) at least one of the two or more microorganisms is capable of having at least one of (i) producing valerate, (ii) producing isovalerate, (iii) reducing nitrate to nitrite, (iv) producing progesterone, (v) reducing nitric oxide, (vi) decreasing methanogenesis, (vii) metabolizing at least one isoflavonoid, (viii) decreasing activity of at least one serine protease, (ix) producing vitamin B6, (x) producing vitamin B12, (xi) pollutants detoxification, or (xii) any combinations thereof.

7-8. (canceled)

9. The microbial consortium of claim 1, wherein said at least one microorganism is capable of having:
(a) one or more of (i) vitamin B12 production, (ii) valerate production, (iii) isovalerate production, (iv) nitrite production, (v) decrease in methane level, (v) decreasing sulphate reduction, or (vi) any combinations thereof,
(b) one or more of (i) putrescine production, (ii) lactate production, (iii) iron sequestering, or (iv) any combinations thereof,
(c) one or more of (i) valerate production, (ii) isovalerate production, (iii) nitrite production, (iv) vitamin B6 production, or (v) any combinations thereof,
(d) one or more of (i) GABA production, (ii) acetate production, (iii) serotonin production or (iv) any combinations thereof,
(e) one or more of (i) decreasing methanogenesis, (ii) decreasing sulphate reduction, or (iii) any combinations thereof,
(f) one or more of (i) vitamin B6 production, (ii) valerate production, (iii) isovalerate production, (iv) decreasing methanogenesis, (vi) decreasing sulphate reduction, or (vii) any combinations thereof, or
(g) at least two of (i) acetate production, (ii) butyrate production, (ii) propionate production, (iv) decreasing methanogenesis, or (v) any combinations thereof.

10-15. (canceled)

16. The microbial consortium of claim 1, wherein said at least one microorganism is capable of (i) increasing serotonin levels, (ii) producing progesterone, or any combinations thereof.

17. (canceled)

18. The microbial consortium of claim 1, wherein at least one of said two or more microorganisms is characterized by having a sequence identity of at least 85% with at least one sequence denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 or SEQ ID NO:40,
optionally wherein at least one of said two or more microorganisms is characterized by having a sequence identity of at least 85% with at least one sequence denoted by SEQ ID NO:1, SEQ ID NO: 2, SEQ ID NO:3, SEQ ID NO:4, SEQ ID NO:5, SEQ ID NO: 6, SEQ ID NO:7, SEQ ID NO:8, or SEQ ID NO:9, and
optionally wherein at least one of said two or more microorganisms is characterized by having a sequence identity of at least 85% with at least three sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO: 9.

19-20. (canceled)

21. The microbial consortium of claim 1, wherein at least one microorganism from said two or more microorganisms is:
(i) at least one of Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486, Megasphaera elsdenii ATCC 25940, Megasphaera sp. MJR8396C, Megasphaera hexanoica JCM 31403, Eubacterium hallii DSM 3353, Acetoanaerobium sticklandii DSM 519, Ruminococcaceae bacterium CPB6, Clostridium kluyveri DSM 555, Coprococcus comes ATCC 27758, Coprococcus catus GD/7, Roseburia inulinivorans DSM 16841, Lactobacillus ruminis ATCC 27782, Lactobacillus rhamnosus GG, Lactobacillus reuteri ATCC 55730, Lactobacillus plantarum subsp. plantarum LB1-2, Lactobacillus salivarius ACS-116-V-Col5a, Dialister succinatiphilus YIT 11850, Bifidobacterium dentium ATCC 27678, Bifidobacterium dentium Bd1, Bifidobacterium adolescentis L2-32, Bifidobacterium adolescentis 22L, Mitsuokella multacida DSM 20544, Selenomonas sputigena ATCC 35185, Phascolarctobacterium succinatutens DSM 22533, Phascolarctobacterium faecium JCM 30894, Oscillibacter sp. PEA192, Eubacterium callanderi KIST612, Acetobacterium woodii DSM 1030, Blautia producta PMF1, Eubacterium callanderi DSM 3662, Eubacterium limosum SA11, or Blautia hydrogenotrophica DSM 10507,
(ii) at least one of Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, or Eubacterium limosum ATCC 8486,
(iii) at least one of Megasphaera elsdenii ATCC 17753, Dialister invisus DSM 15470, Megamonas funiformis YIT 11815, or Eubacterium limosum ATCC 8486,
(iv) at least one of Megasphaera elsdenii ATCC 17753, Lactobacillus ruminis ATCC 25644, Bifidobacterium dentium ATCC 27679, Hafnia alvei ATCC 51873, or Eubacterium limosum ATCC 8486,
(v) at least one of Megasphaera elsdenii ATCC 17753, Dialister invisus DSM 15470, Megamonas funiformis YIT 11815, Lactobacillus ruminis ATCC 25644, Bifidobacterium dentium ATCC 27679, Hafnia alvei ATCC 51873, or Eubacterium limosum ATCC 8486 or (vi) at least one of Megasphaera elsdenii ATCC 17753 or Eubacterium limosum ATCC 8486.

22. The microbial consortium of claim 1, wherein said two or more microorganisms are selected from:
(i) Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486, Megasphaera elsdenii ATCC 25940, Megasphaera sp. MJR8396C, Megasphaera hexanoica JCM 31403, Eubacterium hallii DSM 3353, Acetoanaerobium sticklandii DSM 519, Ruminococcaceae bacterium CPB6, Clostridium kluyveri DSM 555, Coprococcus comes ATCC 27758, Coprococcus catus GD/7, Roseburia inulinivorans DSM 16841, Lactobacillus ruminis ATCC 27782, Lactobacillus rhamnosus GG, Lactobacillus reuteri ATCC 55730, Lactobacillus plantarum subsp. plantarum LB1-2, Lactobacillus salivarius ACS-116-V-Col5a, Dialister succinatiphilus YIT 11850, Bifidobacterium dentium ATCC 27678, Bifidobacterium dentium Bd1, Bifidobacterium adolescentis L2-32, Bifidobacterium adolescentis 22L, Mitsuokella multacida DSM 20544, Selenomonas sputigena ATCC 35185, Phascolarctobacterium succinatutens DSM 22533, Phascolarctobacterium faecium JCM 30894, Oscillibacter sp. PEA192, Eubacterium callanderi KIST612, Acetobacterium woodii DSM 1030, Blautia producta PMF1, Eubacterium callanderi DSM 3662, Eubacterium limosum SA11, or Blautia hydrogenotrophica DSM 10507,
(ii) Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, or Eubacterium limosum ATCC 8486,
(iii) Megasphaera elsdenii ATCC 17753, Dialister invisus DSM 15470, Megamonas funiformis YIT 11815, or Eubacterium limosum ATCC 8486,
(iv) Megasphaera elsdenii ATCC 17753, Lactobacillus ruminis ATCC 25644, Bifidobacterium dentium ATCC 27679, Hafnia alvei ATCC 51873, or Eubacterium limosum ATCC 8486, or
(v) Megasphaera elsdenii ATCC 17753, Dialister invisus DSM 15470, Megamonas funiformis YIT 11815, Lactobacillus ruminis ATCC 25644, Bifidobacterium dentium ATCC 27679, Hafnia alvei ATCC 51873, or Eubacterium limosum ATCC 8486.

23-31. (canceled)

32. The microbial consortium of claim 1 comprising (i) Megasphaera elsdenii ATCC 17753 and Eubacterium limosum ATCC 8486, or (ii) Hafnia alvei ATCC 51873, or combinations thereof.

33-37. (canceled)

38. A pharmaceutical composition comprising the microbial consortium of claim 1 and optionally further comprising carriers, diluents and excipients.

39-44. (canceled)

45. A method for treating, preventing, ameliorating, reducing or delaying the onset of a functional gastrointestinal disorder in a subject in need thereof comprising administering to said subject a therapeutically effective amount of a microbial consortium, wherein said microbial consortium comprises two or more microorganisms, said two or more microorganisms are capable of having at least five of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels, or (xxix) any combinations thereof.

46. The method of claim 45, said microbial consortium having at least eight of (i) putrescine production, (ii) glutamate production, (iii) vitamin B6 production, (iv) vitamin B12 production, (v) arginine production, (vi) GABA production, (vii) acetate production, (viii) butyrate production, (ix) propionate production, (x) valerate production, (xi) isovalerate production, (xii) caproate production, (xiii) linoleic acid production, (xiv) decreasing activity of at least one serine protease, (xv) lactate production, (xvi) increasing iron sequestering, (xvii) secondary bile acid production, (xviii) pollutants detoxification, (xix) decreasing methanogenesis, (xx) decreasing sulphate reduction, (xxi) Treg induction, (xxii) ammonia production, (xxiii) nitrite production, (xxiv) nitric oxide degradation, (xxv) estrogen production, (xxvi) progesterone production (xxvii) isoflavonoids metabolism, (xxviii) increase serotonin levels, or (xxix) any combinations thereof.

47. The method of claim 45, wherein said at least one microorganism is capable of having:
(a) one or more of (i) vitamin B12 production, (ii) valerate production, (iii) isovalerate production, (iv) nitrite production, (v) decrease in methane level, (v) decreasing sulphate reduction, or (vi) any combinations thereof,
(b) one or more of (i) putrescine production, (ii) lactate production, (iii) iron sequestering, or (iv) any combinations thereof,
(c) one or more of (i) valerate production, (ii) isovalerate production, (iii) nitrite production, (iv) vitamin B6 production, or (v) any combinations thereof,
(d) one or more of (i) GABA production, (ii) acetate production, (iii) serotonin production, or (iv) any combinations thereof,
(e) one or more of (i) decreasing methanogenesis, (ii) decreasing sulphate reduction, or (iii) any combinations thereof,
(f) one or more of (i) vitamin B6 production, (ii) valerate production, (iii) isovalerate production, (iv) decreasing methanogenesis, (vi) decreasing sulphate reduction, or (vii) any combinations thereof, or
(g) at least two of (i) acetate production, (ii) butyrate production, (ii) propionate production, (iv) decreasing methanogenesis, or (v) any combinations thereof.

48. The method of claim 45, wherein said at least one microorganism is capable of (i) increasing serotonin levels, (ii) producing progesterone, or any combination thereof.

49. The method of claim 45, wherein at least one of said two or more microorganisms is characterized by having a sequence identity of at least 85% with at least one sequence denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, optionally wherein at least one of said two or more microorganisms is characterized by having a sequence identity of at least 85% with at least one sequence denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9, optionally wherein at least one of said two or more microorganisms is characterized by having a sequence identity of at least 85% with at least three sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

50. The method of claim 45, wherein at least one microorganism from said two or more microorganisms is:
(i) at least one of Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486, Megasphaera elsdenii ATCC 25940, Megasphaera sp. MJR8396C, Megasphaera hexanoica JCM 31403, Eubacterium hallii DSM 3353, Acetoanaerobium sticklandii DSM 519, Ruminococcaceae bacterium CPB6, Clostridium kluyveri DSM 555, Coprococcus comes ATCC 27758, Coprococcus catus GD/7, Roseburia inulinivorans DSM 16841, Lactobacillus ruminis ATCC 27782, Lactobacillus rhamnosus GG, Lactobacillus reuteri ATCC 55730, Lactobacillus plantarum subsp. plantarum LB1-2, Lactobacillus salivarius ACS-116-V-Col5a, Dialister succinatiphilus YIT 11850, Bifidobacterium dentium ATCC 27678, Bifidobacterium dentium Bd1, Bifidobacterium adolescentis L2-32, Bifidobacterium adolescentis 22L, Mitsuokella multacida DSM 20544, Selenomonas sputigena ATCC 35185, Phascolarctobacterium succinatutens DSM 22533, Phascolarctobacterium faecium JCM 30894, Oscillibacter sp. PEA192, Eubacterium callanderi KIST612, Acetobacterium woodii DSM 1030, Blautia producta PMF1, Eubacterium callanderi DSM 3662, Eubacterium limosum SA11, or Blautia hydrogenotrophica DSM 10507,
(ii) at least one of Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, or Eubacterium limosum ATCC 8486,
(iii) at least one of Megasphaera elsdenii ATCC 17753, Dialister invisus DSM 15470, Megamonas funiformis YIT 11815, or Eubacterium limosum ATCC 8486,
(iv) at least one of Megasphaera elsdenii ATCC 17753, Lactobacillus ruminis ATCC 25644, Bifidobacterium dentium ATCC 27679, Hafnia alvei ATCC 51873, or Eubacterium limosum ATCC 8486,
(v) at least one of Megasphaera elsdenii ATCC 17753, Dialister invisus DSM 15470, Megamonas funiformis YIT 11815, Lactobacillus ruminis ATCC 25644, Bifidobacterium dentium ATCC 27679, Hafnia alvei ATCC 51873, or Eubacterium limosum ATCC 8486, or
(vi) at least one of Megasphaera elsdenii ATCC 17753 or Eubacterium limosum ATCC 8486.

51. The method of claim 45, wherein said two or more microorganisms are selected from:
(i) Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, Eubacterium limosum ATCC 8486, Megasphaera elsdenii ATCC 25940, Megasphaera sp. MJR8396C, Megasphaera hexanoica JCM 31403, Eubacterium hallii DSM 3353, Acetoanaerobium sticklandii DSM 519, Ruminococcaceae bacterium CPB6, Clostridium kluyveri DSM 555, Coprococcus comes ATCC 27758, Coprococcus catus GD/7, Roseburia inulinivorans DSM 16841, Lactobacillus ruminis ATCC 27782, Lactobacillus rhamnosus GG, Lactobacillus reuteri ATCC 55730, Lactobacillus plantarum subsp. plantarum LB1-2, Lactobacillus salivarius ACS-116-V-Col5a, Dialister succinatiphilus YIT 11850, Bifidobacterium dentium ATCC 27678, Bifidobacterium dentium Bd1, Bifidobacterium adolescentis L2-32, Bifidobacterium adolescentis 22L, Mitsuokella multacida DSM 20544, Selenomonas sputigena ATCC 35185, Phascolarctobacterium succinatutens DSM 22533, Phascolarctobacterium faecium JCM 30894, Oscillibacter sp. PEA192, Eubacterium callanderi KIST612, Acetobacterium woodii DSM 1030, Blautia producta PMF1, Eubacterium callanderi DSM 3662, Eubacterium limosum SA11, or Blautia hydrogenotrophica DSM 10507,
(ii) Megasphaera elsdenii ATCC 17753, Lactobacillus ruminis ATCC 25644, Dialister invisus DSM 15470, Bifidobacterium dentium ATCC 27679, Eubacterium siraeum DSM 15702, Megamonas funiformis YIT 11815, Oscillibacter valericigenes DSM 18026, Hafnia alvei ATCC 51873, or Eubacterium limosum ATCC 8486,
(iii) Megasphaera elsdenii ATCC 17753, Dialister invisus DSM 15470, Megamonas funiformis YIT 11815, or Eubacterium limosum ATCC 8486,
(iv) Megasphaera elsdenii ATCC 17753, Lactobacillus ruminis ATCC 25644, Bifidobacterium dentium ATCC 27679, Hafnia alvei ATCC 51873, or Eubacterium limosum ATCC 8486, or
(v) Megasphaera elsdenii ATCC 17753, Dialister invisus DSM 15470, Megamonas funiformis YIT 11815, Lactobacillus ruminis ATCC 25644, Bifidobacterium dentium ATCC 27679, Hafnia alvei ATCC 51873, or Eubacterium limosum ATCC 8486.

52. The method of claim 45 comprising (i) Megasphaera elsdenii ATCC 17753 and Eubacterium limosum ATCC 8486, (ii) Hafnia alvei ATCC 51873, or combinations thereof.

53. The method of claim 45, wherein said functional gastrointestinal disorder is a functional dyspepsia, a centrally mediated disorders of gastrointestinal pain or a bowel disorder.

54. The method of claim 53, wherein the bowel disorder is at least one of:
(i) irritable bowel syndrome (IBS), functional constipation, functional diarrhea, functional abdominal bloating/distension, unspecified functional bowel disorder, opioid-induced constipation or any combination thereof, or
(ii) irritable bowel syndrome (IBS), functional constipation, functional diarrhea, functional abdominal bloating/distension, unspecified functional bowel disorder, opioid-induced constipation or any combination thereof, optionally the bowel disorder is one or more of IBS with predominant constipation (IBS-C), IBS with predominant diarrhea (IBS-D), IBS with mixed bowel
